(19) United States
(12) Patent Application Publication
(10) Pub. No.: US 2023/0035690 A1
(43) Pub. Date: Feb. 2, 2023

(54) MACHINE LEARNING TOOLS AND A PROCESS TO DISCOVER NEW NATURAL PRODUCTS BY LINKING GENOMES AND METABOLOMES IN FUNGI

(71) Applicant: Northwestern University, Evanston (IL)

(72) Inventors: Matthew T. Robey, Skokie, IL (US); Paul M. Thomas, Morton Grove, IL (US); Neil L. Kelleher, Evanston, IL (US)

(21) Appl. No.: 17/775,187

(22) PCT Filed: Nov. 6, 2020
(86) PCT No.: PCT/US2020/059502

§ 371 Date: May 6, 2022

Related U.S. Application Data
(60) Provisional application No. 62/932,128, filed on Nov. 7, 2019.

(51) Int. Cl. G16B 5/00; G16B 15/00; G16B 20/00; G16B 40/20; G01N 30/72

(52) U.S. Cl. CPC G16B 5/00; G16B 15/00; G16B 20/00; G16B 40/20; G01N 30/7233; G01N 2030/027

(57) ABSTRACT

Provided herein are method of analyzing genomic and metabolomic data from fungi to identify relationships between biosynthetic gene clusters and mass spectrometric features of metabolites.

### CROSS-REFERENCE TO RELATED APPLICATION

The present application claims priority to U.S. Provisional Patent Application Ser. No. 62/362,437 filed Jul. 14, 2016, which is hereby incorporated by reference in its entirety.

### FIELD

Provided herein are method of analyzing genomic and metabolomic data from fungi to identify relationships between biosynthetic gene clusters and mass spectrometric features of metabolites.

### BACKGROUND

Metabolites from fungi have historically been an invaluable source of therapeutics, including compounds such as penicillin, lovastatin, and cyclosporine. Advances in genome sequencing have revealed that a wealth of new compounds awaits discovery in fungal genomes. Despite the vast potential of fungi for therapeutic development, there is a lack of tools that combine advances in big data analytics, “-omics” biology, and artificial intelligence for large-scale discovery. Standard approaches rely on a “bioactivity-guided” approach that typically results in rediscovery of known compounds.

### SUMMARY

Provided herein are method of analyzing genomic and metabolomic data from fungi to identify relationships between biosynthetic gene clusters and mass spectrometric features of metabolites.

The present platform combines genomics, metabolomics, and machine learning for systematic discovery of new therapeutics from microbes (e.g., fungi). We have previously derisked the Metabologenomics process in actinobacteria (MicroMGx). Systems and methods herein find use in drug discovery, agrochemicals and agricultural biocontrol, fungal pathogen identification and characterization, etc. The present approach instead relies on genomics, metabolomics, and machine learning. Others have used synthetic biology approaches involving extensive manipulations of DNA that are expensive, not scalable, and are challenging to implement in unstudied fungal species. The present approach relies on native producers of natural products and requires no DNA manipulations.

The natural world has provided humanity with a plethora of molecules that have allowed major advances in modem medicine and agriculture. Fungi are one of most prolific providers of these chemicals—yet remain understudied compared to bacteria. With often over 50 natural product biosynthetic gene clusters (BGCs) per strain, fungi contain a potential wealth of new molecules ready to exploit in research. Provided herein is a scalable platform to identify fungal natural products through a fruitful union of bioinformatics, genomics and metabolomics. Provided herein is a “metabologenomics” platform, applied to strain collections of >1000 strains ofActinomycete bacteria, that involves prediction of BGCs from genome sequence data, clustering into gene cluster families (GCFs), collection of large-scale metabolomics data, and correlation of gene cluster families to metabolites. Additionally, in some embodiments the platforms herein utilize machine learning algorithms utilizing custom Hidden Markov Models and random forest classifiers to improve the precision of bioinformatic tools for BGC and GCF annotation, thereby creating a custom fungal-informatic ecosystem that is portable to any strain collection. Experiments were conducted during development of embodiments herein to demonstrate the feasibility of the pipeline herein through a study on nearly 100 sequenced and unsequenced fungal strains. Experiments establish the background library of fungal biosynthetic potential through the meta-analysis of 1,000 publicly available sequenced fungal genomes and then use this library to correlate metabolites to gene clusters for 75 sequenced fungal strains. In some embodiments, provided herein are tools for prioritization of fungal strains for sequencing and application of the pipeline to the metabolites produced by 12 unsequenced strains, sequencing the five most biosynthetically diverse.

The technology utilizes a large-scale correlative approach for connecting biosynthetic pathways encoded in fungal genomes with the metabolites that these pathways produce. The input to the platform is a fungal strain collection. These strains are subjected to broad metabolomics analysis by liquid chromatography-mass spectrometry and whole genome sequencing (if their genomic sequences are unavailable). The pipeline involves a series of informatics steps.

In some embodiments, provide herein are methods and systems utilizing biosynthetic networking and machine learning predictions to analyze fungal genomic sequences to identify BGCs, perform pairwise comparisons of structural and sequence characteristics of BGCs, group BGCs into GCFs, predict molecular substrates for enzymes produced by GCFs and/or BGCs, and/or link GCFs and/or BGCs with product metabolites and/or mass spectrometric features. In some embodiments, a series of bioinformatics algorithms organize predicted biosynthetic pathways into a graph structure based on their similarity. In some embodiments, a machine learning model is used to predict the substrates of enzymes within these pathways, allowing for prediction of metabolite structure.

In some embodiments, provided herein are systems and methods utilizing metabolomics networking and machine learning predictions to analyze mass spectra of fungal metabolite extracts, perform pairwise comparisons mass spectral features between mass specta, group mass spectrometric features into molecular families (MFs), group metabolites into MFs, etc. In some embodiments, the metabolomics approach uses algorithms for organizing mass spectrometry spectral data into a graph structure based on their similarity. These clustered spectra are input into a machine learning model that predicts metabolite structural features.

In some embodiments, provided herein are methods and systems for connecting biosynthetic pathways to metabolites. In some embodiments, a whole-library approach is used for correlating clusters of biosynthetic pathways with spectral nodes in a metabolomics network. In some embodiments, methods and systems herein identify causal relationships between biosynthetic pathways and metabolites, allowing for their targeted discovery for downstream commercial applications including small molecule discovery for both pharmaceutical (human, veterinary) and agrochemical purposes.

In some embodiments, provided herein are methods of combined genomic and metabolomic analysis comprising: (a) analyzing genomic sequences from multiple strains of fungi to generate a network of biosynthetic gene clusters (BGCs); (b) analyzing mass spectra of extracts from multiple strains of fungi to generate a network of metabolite features; and (c) comparing the network of BGCs and network of metabolites to link particular mass spectrometric features with the BGCs responsible for the synthesis of metabolites that correspond to the particular mass spectrometric features.

In some embodiments, the genomic sequences from multiple strains of fungi comprise 100 or more (e.g., 100, 200, 500, 1000, 1500, 2000, 5000, or more, or ranges therebetween) full or partial genomic sequences. In some embodiments, the genomic sequences from multiple strains of fungi comprise full or partial genomic sequences from 100 or more (e.g., 100, 200, 500, 1000, 1500, 2000, 5000, or more, or ranges therebetween) different strains and/or species of fungi. In some embodiments, the genomic sequences from multiple strains of fungi comprise full or partial genomic sequences from 10 or more (e.g., 10, 20, 50, 100, 150, 200, 500, or more, or ranges therebetween) different genera and/or families of fungi. In some embodiments, analyzing genomic sequences from multiple strains of fungi comprises identifying BGCs with the genomic sequences. In some embodiments, analyzing genomic sequences from multiple strains of fungi comprises grouping BGCs with the genomic sequences into gene cluster families (GCFs). In some embodiments, analyzing genomic sequences from multiple strains of fungi is based on pairwise comparisons of sequence and/or predicted structural features of the BGCs.

In some embodiments, the mass spectra of extracts from multiple strains of fungi comprise 100 or more (e.g., 100, 200, 500, 1000, 1500, 2000, 5000, or more, or ranges therebetween) mass spectra. In some embodiments, the mass spectra of extracts from multiple strains of fungi comprise mass spectra from 100 or more (e.g., 100, 200, 500, 1000, 1500, 2000, 5000, or more, or ranges therebetween) strains or species of fungi. In some embodiments, the mass spectra of extracts from multiple strains of fungi comprise mass spectra from 10 or more (e.g., 10, 20, 50, 100, 150, 200, 500, or more, or ranges therebetween) genera or families of fungi. In some embodiments, analyzing mass spectra of extracts from multiple strains of fungi comprises identifying mass spectrometric features with the mass spectra. In some embodiments, analyzing mass spectra of extracts from multiple strains of fungi comprises grouping mass spectrometric features with the mass spectra into molecular families (MFs). In some embodiments, analyzing mass spectra of extracts from multiple strains of fungi is based on pairwise comparisons of mass spectrometric features of the mass spectra.

In some embodiments, comparing the network of BGCs and network of metabolite features comprises comparing the pairwise distances of BGCs or GCFs within the BGC network with the pairwise distances of metabolite features or MFs within the metabolite feature network to identify correlations that indicate that a BGC or GCF is responsible for the synthesis of a metabolite feature or MF. In some embodiments, comparing the network of BGCs and network of metabolite features comprises comparing the frequency of BGCs or GCFs within the BGC network with the frequency of metabolite features or MFs within the metabolite feature network to identify correlations that indicate that a BGC or GCF is responsible for the synthesis of a metabolite feature or MF.

In some embodiments, provided herein are networks linking metabolite features from 100 or more (e.g., 100, 200, 500, 1000, 1500, 2000, 5000, or more, or ranges therebetween) mass spectra of extracts from multiple strains of fungi with BGCs from 100 or more (e.g., 100, 200, 500, 1000, 1500, 2000, 5000, or more, or ranges therebetween) genomic sequences from multiple strains of fungi, wherein linking of a mass spectrometric feature with a BGC indicates that the BGC is involved in the synthesis of a metabolite that produced the mass spectrometric feature.

In some embodiments, provided herein are methods of fungal genomic analysis comprising: (a) identifying biosynthetic gene clusters (BGCs) within genomic sequences from multiple strains of fungi; (b) identifying sequence characteristics and predicted structural domains within the BGCs; and (c) comparing the sequence characteristics and predicted structural domains between multiple pairs of BGCs to determine the degree of relatedness between the pairs of BGCs. In some embodiments, methods further comprise generating a network of BGCs based on the degree of relatedness between the pairs of BGCs. In some embodiments, methods further comprise generating grouping the BGCs into gene cluster families based on the degree of relatedness between the pairs of BGCs.

In some embodiments, provided herein are methods of fungal metabolomic analysis comprising: (a) identifying mass spectrometric features within mass spectra of extracts from multiple strains of fungi; (b) comparing characteristics of the mass spectrometric features between multiple pairs of mass spectrometric features to determine the degree of relatedness between the pairs of mass spectrometric features; and (c) generating a network of mass spectrometric features based on the degree of relatedness between the pairs of mass spectrometric features. In some embodiments, methods further comprise grouping the mass spectrometric features into molecular families based on the degree of relatedness between the pairs of mass spectrometric features.

### BRIEF DESCRIPTION OF THE DRAWINGS

FIG. 1. Exemplary Fungal Artificial Chromosome-Metabolite Scoring (FAC-MS) platform for discovering fungal secondary metabolites originating from unusual biosynthetic gene clusters.

FIG. 2. Proposed terreazepine biosynthetic pathway. a) The terreazepine biosynthetic gene cluster. b) Mass spectral shifts of terreazepine following feeding with D₅-tryptophan and ¹³C₆-anthranilate. c) Proposed incorporation of isotope-labeled precursors into terreazepine. d) selected ion chromatograms of terreazepine in tzpA domain deletion mutants e) Proposed NRPS assembly of terreazepine. It remains unclear if the final cyclization event can occur from both T₂ and T₃ domains.

FIG. 3. MS² fragmentation spectra for terreazepine, fragmented through HCD at a normalized collision energy of 25.0%.

FIG. 4. Overlapping 1H NMR spectra for natural (top) and synthetic (bottom) terreazepine in DMSO-d6. 1H signals are consistent between samples, indicating that the correct product was obtained through synthesis.

FIG. 5. SFC Results for (a) the acylated terreazpine racemic mixture, (b) acylated synthetic (S)-enantiomer, (c) acylated synthetic (R)-enantiomer, (d) and acylated natural terreazepine.

FIG. 6. Selected ion chromatograms of terreazepine in FAC control (top) and tzpB deletion mutants (bottom). The very low production of terreazepine in the deletant strain confirms the involvement of the IDO in terreazepine production.

FIG. 7. (a) Phylogenetic Tree of IDOs in a subset of Aspergilli. IdoA, idoB, and idoC homologs form distinct clades, as annotation according to reference sequences from A. fumigatus and A. oryzae. Interestingly, tzpB and other duplicated IDOs cluster together and share moderate sequence homology to both idoA and idoB. (b) average IDO counts in Aspergilli.

FIG. 8. Diversity of indoleamine 2,3 diooxygenase (IDO)-containing BGCs across fungi. a) Gene cluster families containing IDOs b) distribution of selected IDO-containing biosynthetic gene clusters across diverse Aspergilli.

FIG. 9. IDO-containing Biosynthetic Gene Clusters in Fungi. These gene clusters encompass a wide range of phylogenetically diverse fungi with diverse backbone gene domain sequences.

FIG. 10. Type I and Type II Primary Metabolism Gene Repurposing Strategies. Green arrows represent biosynthetic genes, including backbone genes, tailoring genes, and their regulatory elements. Grey arrows represent hypothetical proteins or genes unrelated to biosynthesis. Yellow arrows found in sterigmatocystin (stc) and echinocandin B (ecd/hty) biosynthetic gene clusters represent examples of Type I repurposing of primary metabolism genes, and red arrows in fellutamide B (inp) and fumagillin (fna) gene clusters represent examples of Type II repurposed primary metabolism genes. FAS=fatty acid synthase, IPMS=isopropylmalate synthase, P-β6=proteasome β6 subunit, M-AP=methionine aminopeptidase.

FIG. 11. Organizing biosynthetic gene clusters (BGCs) from 1037 fungal genomes. (A) Exploring fungal diversity using networks of gene cluster families (GCFs) and molecular families (MFs). A GCF is a collection of similar BGCs aggregated into a network and predicted to use a similar chemical scaffold and create a family of related metabolites. A MF is a collection of metabolites that likewise represent chemical variations around a chemical scaffold. This networking approach enables hierarchical analysis of BGCs and their encoded metabolite scaffolds from large numbers of interpreted genomes. (B) Distribution of BGCs across the fungal kingdom. The BGC content of fungal genomes varies dramatically with phylogeny. Organisms within Pezizomycotina have more BGCs per genome and a greater diversity of biosynthetic types than organisms in Basidiomycota and non-Dikarya phyla.

FIG. 12. The distribution of 12,067 gene cluster families (GCFs) across the fungal kingdom. (A) Heatmap of GCFs across Fungi. The phylogram to the left shows a Neighbor Joining species tree based on 290 shared orthologous genes across 1037 genomes; horizontal shaded regions across the heatmap correspond to each labeled taxonomic group. The order of GCF columns is the result of hierarchical clustering based on the GCF presence/absence matrix. Across Fungi, the distribution of GCFs largely follows phylogenetic trends, with most GCFs confined to a specific genus or species. (B) Relationship between genetic distance and GCF content. The dotted lines indicate median genetic distance values for organisms within the same species, genus, order, class, or phylum. Each point in the scatterplot represents a pair of genomes and the fraction of the pair's GCFs that are shared. (C) Relationship between taxonomic rank and shared GCF content across the fungal kingdom. Violin plots show the fraction of GCFs shared between all pairs of organisms within our 1000-genome dataset, with each pair classified based on the lowest taxonomic rank shared between the two organisms.

FIG. 13. Large-scale analysis of fungal genome-encoded and known metabolite scaffolds. (A) Colliding large scale collections of fungal genetic content (at left) and fungal natural products (at right) using a network of gene cluster families (GCFs) interpreted from 1037 genomes (left) and 15,213 metabolites arranged into 2945 molecular families based on their Tanimoto similarity score (at right). Note that 92% of these 12,067 GCFs remain unassigned to their metabolite products. (B) Variations in adenylation domain substrate-binding residues and tailoring enzyme composition facilitate modifications to the equisetin GCF (left) and MF (right). The phylogram to the left represents a maximum likelihood tree based on the hybrid NRPS-PKS backbone enzyme. All branches in this tree have >50% bootstrap support.

FIG. 14. Fungal biosynthetic gene clusters are distinct from their canonical bacterial counterparts. (A) Principle Component Analysis (PCA) of 36,399 fungal and 24,024 bacteria biosynthetic gene clusters (BGCs), with points sized according to the number of BGCs analyzed. Fungal and bacterial taxonomic groups occupy distinct regions of this biosynthetic space. (B) Fungal and bacterial BGCs differ in backbone enzyme composition, with fungal NRPS and PKS clusters typically encoding only a single backbone, compared to multiple backbone enzymes found in bacterial BGCs. (C) Fungal and bacterial NRPS BGCs differ dramatically in their use of termination domains for release of peptide intermediates. (D) Fungal NRPS logic is distinct from bacterial canon. Most fungal NRPS pathways involve a single NRPS enzyme that utilizes a terminal condensation domain to produce a cyclic peptide. In contrast, bacterial NRPS enzymes contain multiple NRPS enzymes that operate in a colinear fashion and typically utilize thioesterase domains to produce linear or cyclic peptides.

FIG. 15. Bacteria and fungi are distinct sources for natural product scaffolds. (A) Principal Component Analysis (PCA) of 24,595 known bacterial and fungal compounds, with points sized according to the number of compounds. Fungal and bacterial taxonomic groups occupy distinct regions in this representation of chemical space for natural products. (B) Quantitative comparison of structural classifications in bacterial vs fungal compounds. (C) Bacteria and fungi represent distinct pools for bioactive compounds and scaffolds. Selected chemical moieties enriched and characteristic of each taxonomic group are highlighted in yellow. The fold enrichment of the chemical moiety is indicated in green, with p-values from a Chi-Squared test indicated.

FIG. 16. Distribution of 1933 gene cluster families (GCFs) across Basidiomycota. The phylogram to the left shows a Neighbor Joining tree based on 290 orthologous genes, with branches with less than 50% bootstrap support collapsed. Genomes are colored by class, according to NCBI taxonomy information. Genomes within Tremellomycetes are largely composed of subspecies of Cryptococcus neoformans and Cryptococcus gatti and show little variation in GCF content. Within other classes of Basidiomycota, the majority of GCFs are species- or genus-specific. Several GCFs are distributed across entire classes or shared by organisms within different classes.

FIG. 17. Distribution of 822 gene cluster families (GCFs) across Leotiomycetes. The phylogram to the left shows a Neighbor Joining tree based on 290 orthologous genes, with branches with less than 50% bootstrap support collapsed. Genomes are colored by taxonomic order, according to NCBI taxonomy information.

FIG. 18. Distribution of 4926 gene cluster families (GCFs) across Eurotiomycetes. The phylogram to the left shows a Neighbor Joining tree based on 290 orthologous genes, with branches with less than 50% bootstrap support collapsed. Genomes are colored by taxonomic order, according to NCBI taxonomy information.

FIG. 19. Distribution of 1176 gene cluster families (GCFs) across Dothideomycetes. The phylogram to the left shows a Neighbor Joining tree based on 290 orthologous genes, with branches with less than 50% bootstrap support collapsed. Genomes are colored by taxonomic order, according to NCBI taxonomy information.

FIG. 20. Distribution of 2884 gene cluster families (GCFs) across Sordariomycetes. The phylogram to the left shows a Neighbor Joining tree based on 290 orthologous genes, with branches with less than 50% bootstrap support collapsed. Genomes are colored by taxonomic order, according to NCBI taxonomy information.

FIG. 21. Relationship between phylogeny and shared gene cluster family (GCF) content. The phylogram to the left shows a Neighbor Joining tree based on 290 orthologous genes, with branches with less than 50% bootstrap support collapsed. Genomes within Pezizomycotina are labeled by taxonomic class, according to NCBI taxonomy information. Other genomes are labeled by subphylum, according to NCBI taxonomy information.

FIG. 22. Relationship between phylogeny and GCFs in six major taxonomic groups. The violin plots represent the fraction of gene cluster families (GCFs) shared by pairs of genomes within the given taxonomic groups. Each genome pair was given a mutually-exclusive classification of same-species, same-genus, or same-class, and the fraction of GCFs shared for each genome pair was determined.

FIG. 23. Fungal gene cluster families (GCFs) are largely species-specific. Each GCF within the given taxonomic group was classified based on highest taxonomic rank shared by organisms with the GCF (i.e. species-specific, genus-specific, family-specific, etc.). Depending on taxonomic group, GCFs are between 68-89% species-specific.

FIG. 24. Using the GCF approach for automated annotation of fungal BGCs with putative metabolite scaffolds. Across the taxonomic groups examined, a total of 154 GCFs contain reference BGCs with known metabolite products. At the level of individual clusters, these amounts to 2,026 BGCs annotated based on their presence in GCFs with known metabolite scaffolds.

FIG. 25. Comparison of metabolite scaffold chemical space covered by molecular families (MFs) and gene cluster families (GCFs). At each clustering threshold, the median Tanimoto similarity of known compounds within GCFs and MFs was determined. A median intra-cluster Tanimoto similarity of 0.7 was chosen, corresponding to GCF and MF similarity thresholds of 0.45 and 0.6, respectively.

FIG. 26. Compounds from the equisetin structural class that have associated known gene clusters. The scaffold includes a hydrocarbon decalin core varying in methyl and alkenyl substituents and stereochemistry. A tetramic acid moiety derived from serine or threonine is conjugated to the decalin core. N-methylation of the tetramic acid amide is present in equisetin and phomasetin.

FIG. 27. The biosynthetic pathway for equisetin and related compounds. First the core decalin ring is constructed by a hybrid nonribosomal peptide synthetase-polyketide synthase (NRPS-PKS) enzyme. The PKS domains within the backbone enzyme act in an iterative fashion typical of fungal PKS enzymes, assembling the decalin core from malonyl-CoA monomers. This step is supplemented by the action of a standalone enoyl reductase for ketide monomer reduction and a Diels-Alderase that directs ring closure and controls stereochemistry (14, 15). Second, an NRPS module condenses an amino acid to the decalin core (16). A terminal reductase domain catalyzes Dieckman cyclization to release the intermediate as a tetramic acid, the third step (17). In the final pathway step, a methyltransferase catalyzes N-methylation of the tetramic acid amide (16).

FIG. 28. Diversification of chemical scaffolds across gene cluster families. The GCF for PR-toxin (TERPENE_139), a DNA polymerase mycotoxin produced by Penicillium roqueforti (18), contains an additional P450 enzyme in a BGC from the Sordariomycete Stachybotrys chartarum. The GCF for chaetoglobosin A, a scaffold with a variety of anti-cancer activities (19), contains a methyltransferase in a BGC from the Dothideomycete Ramularia collo-cygni not present in the experimentally-characterized BGC from Penicillium expansum. The GCF for swainsonine (HYBRIDS_151), an α-mannosidase inhibitor advanced to clinical trials as a potential anti-cancer therapeutic (20, 21), contains variable F420 oxidoreductase, short chain dehydrogenase, and an NAD oxidoreductase, and aminotransferase enzymes. In the GCF for cytochalasin E (HYBRIDS_197), a compound with anticancer activity, BGCs differ in the presence/absence of a pyridine oxidoreductase and an FAD oxidoreductase present in the experimentally-characterized Aspergillus clavatus BGC.

FIG. 29. Comparison of fungal and bacterial NRPS and PKS backbone sizes. For both NRPS and PKS enzymes, fungal backbones are longer both in terms of amino acids and catalytic domains per backbone enzyme.

FIG. 30. Comparison of fungal and bacterial NRPS domain organizations. In fungi (top), the most common NRPS domain organizations include terminal condensation or thioester reductase domains. Fungal NRPS enzymes also commonly employ iterative modules. In bacteria, the most common NRPS domain organizations feature terminal thioesterase domains and/or N-terminal condensation domains that interact with an upstream NRPS enzyme catalyze N-acylation.

FIG. 31. PCA plot (left) and associated loadings plot (right) of bacterial and fungal chemical space. Fungal and bacterial taxonomic groups represent distinct regions in this space. Fungi are distinguished from bacteria due to an increased frequency of chemical ontology terms associated with aromatic polyketides, such as anisoles, ketones, and alkyl aryl ethers. Bacteria are distinguished largely due to peptide-associated chemical ontology terms (i.e. organic acids, azacyclics, amides).

FIG. 32. PCA analysis of fungal chemical space. Eurotiomycetes, Sordariomycetes, Dothideomycetes, and Leotiomycetes (Ascomycota) are distinct largely based on polyketide and peptide-related chemical ontology terms, such as azacyclic, Oxacyclic, Benzenoids, and Lactams. Lipid-associated chemical ontology terms are prevalent in Basidiomycota and Mucoromycota.

FIG. 33. Breakdown of chemical superclasses in fungal taxa. The chemical space of distinct fungal taxonomic groups varies dramatically. Basidiomycota and Mucoromycota are both ˜50% lipids. Other taxonomic groups contain a higher fraction of organoheterocyclic compounds.

FIG. 34. PCA plot (left) and associated loading plot (right) of biosynthetic domains contained within NRPS-containing biosynthetic gene clusters. Chytridiomycota are pulled in the positive direction on the x-axis due to their high frequency of large NRPS backbone enzymes containing many adenylation, condensation, and thiolation domains, while Pezizomycotina are largely pulled in the “up” direction due to the presence of NRPS-PKS hybrids.

FIG. 35. PCA plot (left) and associated loading plot (right) of biosynthetic domains contained within PKS-containing biosynthetic gene clusters. Eurotiomycetes, Leotiomycetes, Dothideomycetes, and Sordariomycetes contain the most PKS backbone enzymes, and are pulled to the right by the corresponding PKS domains. Several regulatory elements are associated with these backbone genes, providing insight into the way fungi regulate PKS biosynthesis.

FIG. 36. A roadmap for sampling Eurotiomycetes genomes for natural products discovery based on shared GCFs. Each curve shows the fraction of Eurotiomycetes GCFs that would be present in genomes sampled using different approaches. All Genomes shows the results of randomly sampling from all 368 Eurotiomycetes genomes. Species and other taxonomic ranks shows the result of randomly sampling unique species, genera, families, or orders. GCF-Based Sampling shows the result of sampling clusters of organisms that share GCFs (“clusters” representing the results of density-based clustering, not biosynthetic gene clusters). The red boxed numbers indicate the number of genomes required reach 80% GCF coverage, the threshold indicated by the dashed red line. Small numbers along each curve indicate the number of genomes randomly sampled from each group. GCF-based sampling of organisms reaches 80% coverage of GCFs after 145 genomes sampled, species-based sampling of organisms requires 189 genomes, and random sampling of all genomes requires 263 genomes to reach this threshold. This indicates that sampling of organisms for biosynthetic pathway and compound discovery based on GCF overlap can provide a more efficient means of accessing these GCFs. Each random sampling of genomes was performed using 1000 iterations.

FIG. 37. Comparison of the pharmacological properties of bacterial (n=9,382), fungal (n=15,213), and FDA-approved compounds (n=2884). Error bars represent 95% confidence intervals determined by bootstrap sampling. Asterisks indicate statistically-significant differences between the means (p<0.01, Student's t-test).

FIG. 38. Determining the optimal genetic marker for predicting fungal GCF similarity. The commonly used ITS sequence and the alternative rpb2 sequence show a poor relationship with GCF similarity; however, benA shows a defined relationship with GCF overlap. The 96-99% identity region will be used to target unsequenced strains with 40-60% overlap in GCF content to known strains.

FIG. 39. Top, Workflow for the gene cluster families (GCFs) approach. Biosynthetic gene clusters from fungal genomes are organized into gene cluster families based on shared domains and sequence identity. Bottom, Network of 594 GCFs for 50 fungi; GCFs in red are annotated based on known gene clusters; unassigned GCFs are in blue.

FIG. 40. Correlation data for known NP/BGC pairs, validating the metabologenomics approach as viable, even using 50 fungal strains.

FIG. 41. A. Appearance of metabolite with m z 343.129 in extracts from 50 fungal strains. Strains with green highlight contain a BGC that belongs to the ‘hybrids_158’ gene cluster family (GCF), and the bars correspond to peak areas of m z 343.129 metabolite from strains grown in four media. B. Target ions for isolation and biosynthetic studies. [*p-values were developed for scoring the frequency of co-occurrence of GCFs and compounds, and were corrected for multiple-hypothesis testing using the conservative Bonferroni method.] C. Gene cluster diagram for the new, associated BGC from A. brasilensis.

### DEFINITIONS

As used herein the term “biosynthetic gene cluster” (“BGC”) refers to a set of several genes that direct the synthesis of a particular metabolite (e.g., a secondary metabolite). The genes are typically located on the same stretch of a genome, often within a few thousand bases of each other. Genes of a BGC may encode proteins which are similar or unrelated in structure and/or function. The encoded proteins are typically either (i) enzymes involved in the biosynthesis of metabolites or metabolite precursors and/or (ii) are involved inter alia in regulation or transport of metabolites or metabolite precursors. Together, the genes of the BGC encode proteins that serve the purpose of the biosynthesis of the metabolite. The term “putative biosynthetic gene cluster” (“pBGC”) refers to a segment of a genome that is suspected of being a BGC or is to be tested for being a BGC. A pBGC may be identified by computational genomic analysis, functional analysis of the genes in a stretch of a genome, other techniques, or combinations thereof.

As used herein, the term “gene cluster family” (“GCF”) refers to a set of two or more biosynthetic gene clusters from one or more genomic sequences (e.g., from the same or different strain, species, genus, etc.) that bear sufficiently similar sequence or structural features (e.g., predicted structural features) to indicate that that the BCGs with in the GCF are involved in or responsible for the synthesis of related metabolites.

As used herein, the term “metabolite” refers to a molecule that is an intermediate or an end product of a metabolic process.

As used herein, the term “primary metabolite” refers to a molecule that is directly involved in normal growth, development, and reproduction of an organism, and is present across the spectrum of cell and organism types. Common examples of primary metabolites include, but are not limited to ethanol, lactic acid, and certain amino acids.

As used herein, the term “secondary metabolite” refers to a molecule that is typically not directly involved in processes central to growth, development, and reproduction of an organism, and is present in a taxonomically restricted set of organisms or cells (e.g., plants, fungi, bacteria, or specific species or genera thereof). Examples of secondary metabolites include ergot alkaloids, antibiotics, naphthalenes, nucleosides, phenazines, quinolines, terpenoids, peptides, and growth factors.

As used herein, the term “small molecule” refers to organic or inorganic molecular species either synthesized or found in nature, generally having a molecular weight less than 10,000 grams per mole, optionally less than 5,000 grams per mole, and optionally less than 2,000 grams per mole.

As used herein, the term molecular family (“MF”) refers to a set of two or more mass spectrometric features from one or more mass spectra (e.g., from the same or different strain, species, genus, etc.), or a set of two or more metabolites from one or metabolite extracts (e.g., from the same or different strain, species, genus, etc.), that bear sufficiently similar mass spectrometric or structural features (e.g., predicted structural features) to indicate that that the mass spectrometric features and/or metabolites within the MF are related or produced by related metabolites.

As used herein, the term “network” refers to a group of nodes (e.g., BGCs, GCFs, MS features, MFs, metabolites, etc.) linked and/or arranged according to the degree of relatedness of the nodes.

### DETAILED DESCRIPTION

Provided herein are method of analyzing genomic and metabolomic data from fungi to identify relationships between biosynthetic gene clusters and mass spectrometric features of metabolites. In some embodiments, provided herein are networks and methods of generating networks of genomic and/or metabolomic analyses.

In some embodiments, provide herein are systems and methods utilizing biosynthetic networking and machine learning predictions, for example, to generate networks of BGCs and GCFs. In some embodiments, fungal genomes are obtained either by whole genome sequencing or through a public database such as GenBank or the Joint Genome Institute's Genome Portal. In some embodiments, biosynthetic gene clusters are identified within these genomes using computational methods (e.g., antiSMASH, an open-source Python program). In some embodiments, a distance metric is applied to pairs of BGCs (e.g., all combination pairs of BGCs in the genome sequences) to construct a biosynthetic network of related gene clusters. In some embodiments, pairs of BGCs with more related sequence and/or predicted structural features (e.g., secondary structures, domains, etc.) receive a small distance score and are closer together within the network. In some embodiments, a distance metric is calculated between every BGC pair in a set of genomic sequences. In some embodiments, a distance metric is calculated based on one or more sub-metrics, such as:
The percent identity of a core biosynthetic domain (e.g., an adenylation, ketosynthase, product template, acyltransferase, or terpene synthase domain, etc.). In some embodiments, in the case of duplicate domains, the most likely pairs of homologous domains are identified using, for example. A Hungarian Matching algorithm, which finds the maximum similarity matchings in a bipartite graph.
The Jaccard similarity of protein domains in the two gene clusters.
The longest common subsequence of protein domain strings from the two gene clusters.
   In some embodiments, the weighted sum of these the sub-metrics metrics is used to calculate a distance metric used for clustering the BGCs in a network. In some embodiments, the result is a graphical representation in which nodes represent gene clusters, edges represent similarity, and subgraphs represent “gene cluster families,” groups of homologous gene clusters likely to encode the same metabolite (or a set of similar metabolites).

In some embodiments, for each non-ribosomal peptide synthetase gene cluster node in the biosynthetic graph, a random forest classifier is used to predict its amino acid substrates. Experiments were conducted during development of embodiments herein to train this model was on 1200 adenylation domain sequences with known substrate specificities.

In some embodiments, provided herein are systems and methods utilizing metabolomics networking and machine learning predictions, for example, to generate networks of mass spectrometric features, predicted metabolites, and molecular families of metabolites and/or MS features. In some embodiments, metabolomics data is collected using liquid chromatography-mass spectrometry on a high-resolution instrument. Fragmentation spectra are extracted from mass spectrometry files. In some embodiments, for metabolomics network creation, consensus spectra are generated from spectra arising from identical metabolites. In some embodiments, spectra with similar precursor m z values (e.g., within 20 ppm, within 15 ppm, within 10 ppm, within 5 ppm, within 2 ppm, within 1 ppm) of each other and a cosine similarity of at least 0.1, 0.2, 0.3, 0.4, 0.5, 0.6, 0.7, 0.8, 0.9, 1.0, etc. (e.g., at least 0.6 ppm) are summed to create a consensus spectrum with much higher signal:noise than the original spectra. In some embodiments, a distance matrix is calculated for all consensus spectra. In some embodiments, spectra are binned into fixed-dimension vectors and a cosine similarity matrix is calculated. In some embodiments, distances within this matrix that meet a threshold requirement are added as edges to a graph. In some embodiments, a pruning step trims each subgraph in the graph to a threshold subgraph size parameter. In some embodiments, provided herein are methods of producing a graphical representation of a network where each node represents a metabolite consensus spectrum, edges represent similarity between spectra, and subgraphs represent clusters of structurally and biosynthetically-related metabolites.

In some embodiments, following metabolomic network creation, a neural network model is used to predict substructural features from each node in the network. In experiments conducted during development of embodiments herein, a neural network was trained using ˜24,000 publicly-available reference spectra. Each spectrum is binned and encoded as a 2000-dimensional vector. Each reference spectrum has an associated chemical structure, which is encoded as a vector of substructures and chemical features determined using the tool ClassyFire. The neural network model, trained using these 24,000 spectra, is composed of a single hidden layer with 1024 nodes, ReLU activation functions for the hidden layer, and an output layer computing a sigmoid activation function for each chemical feature. This neural network model thus enables structural predictions for spectral nodes with the metabolomics network.

In some embodiments, provided herein are methods and systems for connecting biosynthetic pathways to metabolites. In some embodiments, correlative statistics are employed for connecting biosynthetic pathways with metabolites. In some embodiments, a correlation matrix is constructed using statistical analysis, for example, a chi-squared test comparing pairwise frequencies of gene cluster family subgraphs from the biosynthetic network with spectral nodes from the metabolomics network. In some embodiments, a Bonferroni correction is used to account for multiple hypothesis testing. In some embodiments, methods provided herein result in a score (e.g., −log₁₀[pvalue]) for each metabolite node-gene cluster family pair, with high scores indicating strong associations. In some embodiments, biosynthetic and metabolomic machine learning predictions are used to identify causal metabolite-gene cluster family pairs.

In some embodiments, a network (e.g., web portal) is utilized to share and/or analyze data produced by the methods herein among researchers (e.g., non-local researchers; at distant locations, etc.).

Prior work has utilized bioactivity-guided fractionation for natural products discovery, rather than a metabolomics, genomics, and machine learning approach. Researchers have focused on synthetic biology and heterologous expression, in contrast to an approach which does not require DNA manipulations. Tools have been developed for clustering metabolomics spectra and performing metabolite machine learning predictions. These tools use different machine learning models and are not integrated into larger genomics workflows. Tools have been developed for predicting adenylation domain substrates and for creating biosynthetic networks from gene clusters; however, these tools are ineffective for fungal genomes. An integrated genomics-metabolomics platform has been developed for natural products discovery; however, this platform is not applicable to fungal genomes.

Systems and method for untargeted metabolomic screening are described, for example in U.S. Pat. No. 10,808,256, which is herein incorporated by reference in its entirety.

### EXPERIMENTAL

### Example 1

### Heterologous Expression of the Terreazepine Biosynthetic Gene Cluster

Fungal natural products (secondary metabolites) are an invaluable source for pharmaceuticals that act against myriad conditions, including infectious diseases, cancer, and hyperlipidemia (Refs A1-A4; incorporated by reference in their entireties). Indeed, the antibiotics penicillin and cephalosporin, the cholesterol-lowering lovastatin, and the immunosuppressant cyclosporine are derived from fungi (Refs. A5, A6; incorporated by reference in their entireties), and the reservoir of novel scaffolds continues to grow each year (Ref. 7; incorporated by reference in its entirety). Although numerous fungi-derived drugs exist on the market today, genome sequencing has revealed that fungi possess the biosynthetic capacity to produce a far greater number of secondary metabolites than currently accessed (Ref. 8; incorporated by reference in its entirety). Recent studies spanning nearly 600 fungal genomes suggest that a mere 3% of molecules encoded by fungal biosynthetic gene clusters (BGCs) have been explored (Ref. 8; incorporated by reference in its entirety).

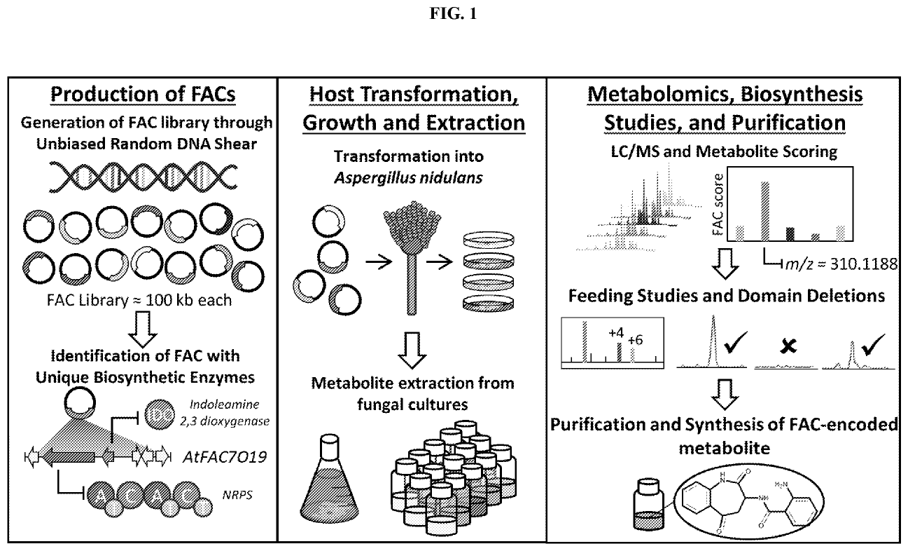

Provided herein are methods comprising a discovery pipeline ntly developed to systematically annotate the biosynthetic abilities of fungi using comparative metabolomics and heterologous gene expression (Refs. A9-A12; incorporated by reference in their entireties). With this platform, fungal genomic DNA fragments containing intact BGCs are inserted into fungal artificial chromosomes (FACs) and transformed into a fungal host to discover new chemical scaffolds (Refs. A10-A12; incorporated by reference in their entireties). The pipeline uses a metabolite scoring (MS) system to identify heterologously-expressed metabolites from the thousands of signals originating from the host. By enabling facile linkage between secondary metabolites and their corresponding BGCs, the FAC-MS pipeline facilitates prioritization of target compounds most likely to contain novel scaffolds. Using structural clues provided by BGC data, compounds originating from BGCs containing unusual biosynthetic machinery are targeted (FIG. 1).

Aromatic amino acids are fundamental for growth and development across phylogenetic kingdoms. Additionally, catabolism of aromatic amino acids leads to the production of non-proteinogenic amino acids, such as the tryptophan-derived kynurenine, which regulates inflammation and immune responses (Refs. A13, A14; incorporated by reference in their entireties). Kynurenine and its derivatives are biosynthetic intermediates of numerous secondary metabolites, including sibiromycin (Ref. A15; incorporated by reference in its entirety), mycemycin C (Ref. A16; incorporated by reference in its entirety), nidulanin A (Ref. A17; incorporated by reference in its entirety), nidulanin B and nidulanin D (Ref A18; incorporated by reference in its entirety), daptomycin (Ref. A19; incorporated by reference in its entirety), and quinomycin peptide antibiotics (Ref. A20; incorporated by reference in its entirety). Incorporation of kynurenine into secondary metabolites enables differential specificity towards enzyme receptors and targets (Ref. A21; incorporated by reference in its entirety). Daptomycin, for example, shows decreased antimicrobial efficacy when kynurenine is mutated to tryptophan (Refs. A22-A23; incorporated by reference in their entireties). One tactic for creating secondary metabolites with novel scaffolds is to recruit primary metabolic enzymes that modify common precursors into non-proteinogenic precursors into BGCs (Ref. A20; incorporated by reference in its entirety). For example, a tryptophan 2,3-dioxygenase (TDO) located adjacent to the daptomycin-producing non-ribosomal peptide synthase (NRPS) supplies the kynurenine for daptomycin synthesis. This TDO diverges from related proteins in the same genus (29% sequence identity), suggesting it is a paralogous enzyme dedicated to secondary metabolite biosynthesis (Ref. A19; incorporated by reference in its entirety).

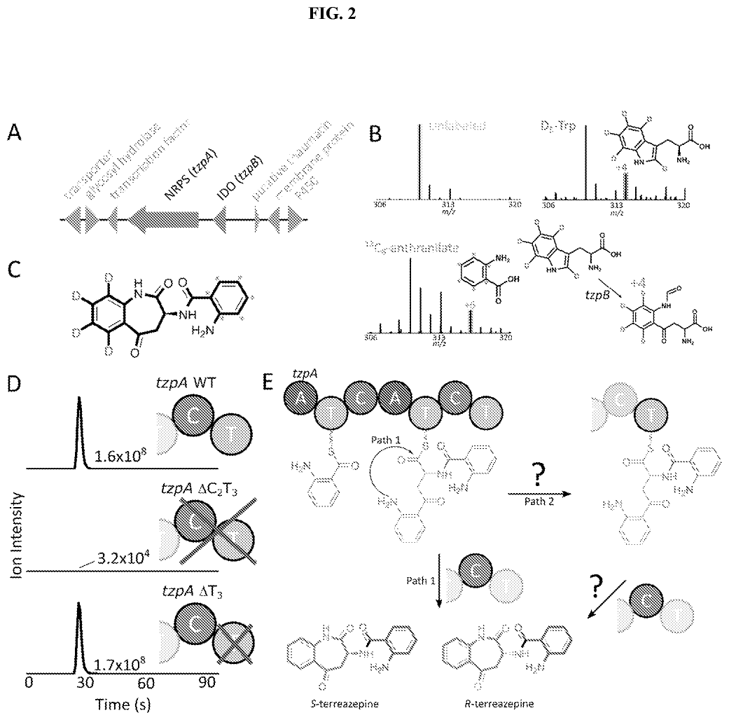

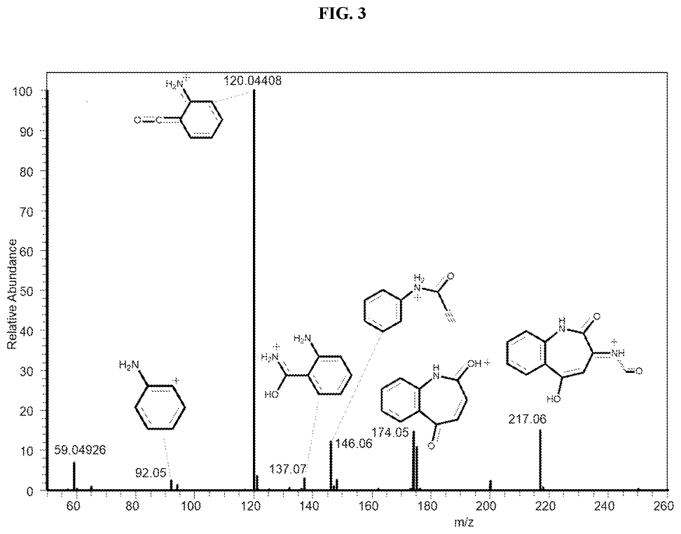

In a large-scale analysis of 56 FACs, an unknown metabolite from heterologous expression of a BGC from Aspergillus terreus ATCC 20542 (located on the FAC AtFAC7O19, FIG. 2A; see also Table 1) was identified with an m/z value of 310.1188 and a molecular formula of C₁₇H₁₅N₃O₃ (10). This compound was found in both the parent strain and the AtFAC7O19-transformed A. nidulans, but not in the empty vector control. The BGC encoding this metabolite contained an indoleamine 2,3-dioxygenase (IDO), which is involved in tryptophan degradation via kynurenine production (Ref. A24; incorporated by reference in its entirety). While most Aspergilli contain three IDOs, A. terreus contains four (FIG. 3). Given that gene duplication is often utilized as a strategy to “repurpose” genes for secondary metabolism, the presence of this fourth IDO suggested that it may serve to supply kynurenine for the formation of the identified secondary metabolite. The FAC-MS strategy was employed in experiments conducted during development of embodiments herein to identify the biosynthetic product of this unusual gene cluster and probe its biosynthesis.

TABLE 1
Annotated Boundaries of AtFAC7O19 in comparison with the A. terreus NIH2624
reference genome.

| | [symbol] | | | [symbol] | | | |
|---|---|---|---|---|---|---|---|
| Gene ID | Start | End | Annotation | Gene ID | Start | End | Annotation |
| FAC38_01 | [symbol] | [symbol] | hypothetical protein | ATEG_07322 | [symbol] | [symbol] | conserved hypothetical protein |
| FAC38_02 | [symbol] | [symbol] | ER membrane protein complex subunit 1 | ATEG_07323 | [symbol] | [symbol] | conserved hypothetical protein |
| FAC38_03 | [symbol] | [symbol] | hypothetical protein | | | | |
| FAC38_05 | [symbol] | [symbol] | [symbol] | ATEG_07324 | [symbol] | [symbol] | predicted protein |
| FAC38_06 | [symbol] | [symbol] | [symbol] | ATEG_07325 | [symbol] | [symbol] | conserved hypothetical protein |
| FAC38_07 | [symbol] | [symbol] | [symbol] | ATEG_07326 | [symbol] | [symbol] | conserved hypothetical protein |
| FAC38_08 | [symbol] | [symbol] | Fatty acid amide hydrolase | ATEG_07327 | [symbol] | [symbol] | conserved hypothetical protein |
| FAC38_09 | [symbol] | [symbol] | [symbol] | ATEG_07328 | [symbol] | [symbol] | conserved hypothetical protein |
| FAC38_10 | [symbol] | [symbol] | hypothetical protein | ATEG_07329 | [symbol] | [symbol] | conserved hypothetical protein |
| FAC38_11 | [symbol] | [symbol] | [symbol] | ATEG_07330 | [symbol] | [symbol] | predicted protein |
| FAC38_12 | [symbol] | [symbol] | [symbol] | ATEG_07331 | [symbol] | [symbol] | [symbol] |
| FAC38_13 | [symbol] | [symbol] | [symbol] | ATEG_07332 | [symbol] | [symbol] | [symbol] |
| FAC38_14 | [symbol] | [symbol] | [symbol] | ATEG_07333 | [symbol] | [symbol] | conserved hypothetical protein |
| FAC38_15 | [symbol] | [symbol] | [symbol] | ATEG_07334 | [symbol] | [symbol] | conserved hypothetical protein |
| FAC38_16 | [symbol] | [symbol] | [symbol] | ATEG_07335 | [symbol] | [symbol] | hypothetical protein |
| FAC38_17 | [symbol] | [symbol] | [symbol] | ATEG_07336 | [symbol] | [symbol] | conserved hypothetical protein |
| FAC38_18 | [symbol] | [symbol] | [symbol] | ATEG_07337 | [symbol] | [symbol] | conserved hypothetical protein |
| FAC38_19 | [symbol] | [symbol] | Acetamidase | ATEG_07338 | [symbol] | [symbol] | similar to general amidase C |
| FAC38_20 | [symbol] | [symbol] | [symbol] | ATEG_07340 | [symbol] | [symbol] | conserved hypothetical protein |
| FAC38_21 | [symbol] | [symbol] | [symbol] | ATEG_07341 | [symbol] | [symbol] | predicted protein |
| FAC38_22 | [symbol] | [symbol] | Lysine/arginine permease | ATEG_07342 | [symbol] | [symbol] | conserved hypothetical protein |
| FAC38_23 | [symbol] | [symbol] | [symbol] | ATEG_07343 | [symbol] | [symbol] | predicted protein |
| FAC38_24 | [symbol] | [symbol] | [symbol] | ATEG_07344 | [symbol] | [symbol] | predicted protein |
| FAC38_25 | [symbol] | [symbol] | [symbol] | ATEG_07345 | [symbol] | [symbol] | [symbol] |
| FAC38_26 | [symbol] | [symbol] | [symbol] | ATEG_07346 | [symbol] | [symbol] | conserved hypothetical protein |
| FAC38_27 | [symbol] | [symbol] | [symbol] | ATEG_07347 | [symbol] | [symbol] | conserved hypothetical protein |
| FAC38_28 | [symbol] | [symbol] | hypothetical protein | none | | | |
| FAC38_29 | [symbol] | [symbol] | [symbol] | none | | | |
| FAC38_30 | [symbol] | [symbol] | [symbol] | none | | | |
| FAC38_31 | [symbol] | [symbol] | [symbol] | ATEG_07354 | [symbol] | [symbol] | [symbol] |
| FAC38_32 | [symbol] | [symbol] | High-affinity glucose transporter [symbol] | ATEG_07355 | [symbol] | [symbol] | sugar transporter |
| FAC38_33 | [symbol] | [symbol] | [symbol] | ATEG_07356 | [symbol] | [symbol] | [symbol] |
| FAC38_34 | [symbol] | [symbol] | [symbol] | ATEG_07357 | [symbol] | [symbol] | fungal specific transcription |
| | | | | | | | factor domain-containing |
| | | | | | | | protein |
| FAC38_35 | [symbol] | [symbol] | [symbol] | ATEG_07358 | [symbol] | [symbol] | [symbol] |
| [symbol] | | | | | | | |
| FAC38_36 | [symbol] | [symbol] | [symbol] | ATEG_07359 | [symbol] | [symbol] | [symbol] |
| [symbol] | | | | | | | |
| FAC38_37 | [symbol] | [symbol] | Thromatin-like protein | ATEG_07360 | [symbol] | [symbol] | extracellular thaumatin |
| | | | | | | | domain protein |
| FAC38_38 | [symbol] | [symbol] | [symbol] | ATEG_07361 | [symbol] | [symbol] | integral membrane protein |
| FAC38_39 | [symbol] | [symbol] | [symbol] | ATEG_07362 | [symbol] | [symbol] | [symbol] |
| FAC38_40 | [symbol] | [symbol] | hypothetical protein | ATEG_07363 | [symbol] | [symbol] | MES transporter |
| FAC38_41 | [symbol] | [symbol] | Kinesin light chain | ATEG_07364 | [symbol] | [symbol] | hypothetical protein |

[symbol]

indicates data missing or illegible when filed

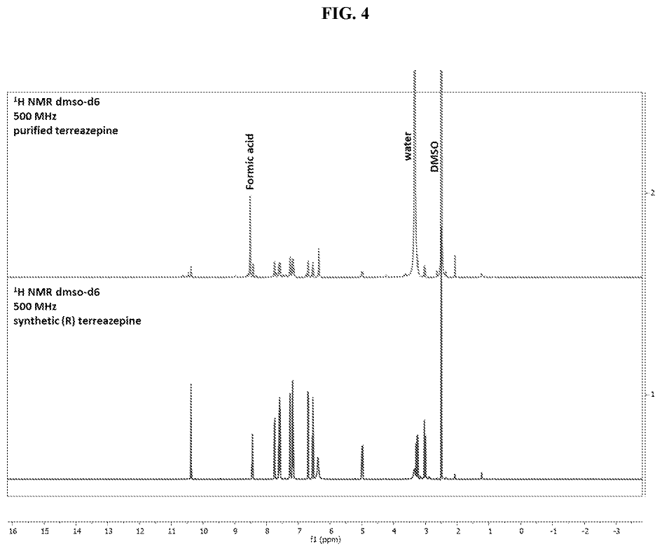

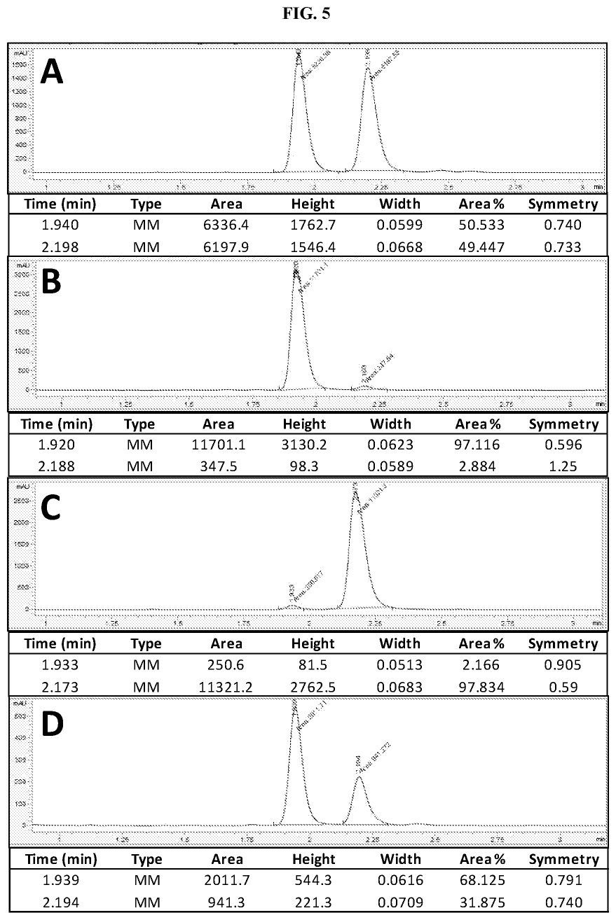

To determine the structure of the target compound, ˜1.5 mg of material was purified from FAC-transformed A. nidulans extracts and subjected to MS² analysis, ¹H and ¹³C NMR spectroscopy, and two-dimensional correlation approaches including COSY, HSQC, and HMBC (Table 2 and FIGS. 3-4). Structural analysis revealed an unusual secondary metabolite backbone, a 3,4-dihydro-TH-1-benzazepine-2,5-dione, resulting from the unusual cyclization of kynurenine. The metabolite's structure matches that of a previously-synthesized kynurenine derivative, 2-amino-N-(2,3,4,5-tetrahydro-2,5-dioxo-1H-1-benzazepin-3-yl)benzamide (Ref. A25; incorporated by reference in its entirety). Based on its structure and the parent organism, it was given a common name of “terreazepine.” To determine the stereochemical configuration of terreazepine, (R) and (S) enantiomers were synthesized, each with an enantiomeric excess ≥95% (FIG. 5). Each enantiomer and the purified natural compound were acylated to enable separation using supercritical fluid chromatography. Natural terreazepine was found to be a 2:1 mixture of S:R enantiomers (FIG. 5). (S)-terreazepine (nanangelenin B) is an intermediate in the biosynthesis of the related compound nanangelenin A (Ref. A26; incorporated by reference in its entirety).

TABLE 2
NMR data for terreazepine in DMSO-d6. 1H, COSY, HMBC, and HSQC data
collected at 500 MHz, and 13C data collected at 125 MHz. Overlapping assignments (*)
were determined using HSQC and HMBC data.

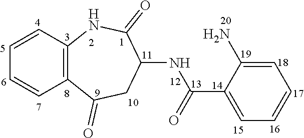 [SMILES: Nc1ccccc1C(=O)NC1CC(=O)c2ccccc2NC1=O]

| Position | ¹³C | ¹H | HMBC | COSY |
|---|---|---|---|---|
| 1 | 171.25 | — | 2, 10α, 10β | — |
| 2 | — | 10.38, s, 1H | — | — |
| 3 | 137.72 | | 5, 7 | — |
| 4 | 122.22 | 7.19, d, J = 8.0, 1H | 2, 6 | 5 |
| 5 | 134.25 | 7.61, td, J = 7.24, 1.42, 1H | 7 | 4, 6 |
| 6 | 124.36 | 7.27, t = 7.57, 1H | 4 | 7, 5 |
| 7 | 130.12 | 7.76, dd, J = 7.88, 1.67, 1H | 5 | 6 |
| 8 | 128.47* | — | 2, 6, 10α | — |
| 9 | 197.76 | — | 7, 10α, 10β, 11 | — |
| 10 | 45.75 | 10α: 3.02, dd, J = 18.7, 2.6, 1H | — | 11 |
| | | 10β: 3.24, dd, J = 18.7, 13.3, 1H | | |
| 11 | 46.14 | 4.99, ddd, J = 13.2, 7.4, 2.5, 1H | 2, 10α, 10β | 10β, 12 |
| 12 | — | 8.42, d, J = 7.42, 1H | | 11 |
| 13 | 168.63 | — | 12, 15 | — |
| 14 | 113.87 | — | 16 | — |
| 15 | 128.47* | 7.58, d, J = 7.6, 1H | 17 | 16 |
| 16 | 114.59 | 6.54, t, J = 7.9, 1H | 18 | 15, 17 |
| 17 | 132.13 | 7.17, m, 1H | 15 | 16, 18 |
| 18 | 118.38 | 6.69, d, J = 8.1, 1H | 16 | — |
| 19 | 149.70 | — | 15, 17 | — |
| 20 | — | 6.38, s, 2 H | — | — |

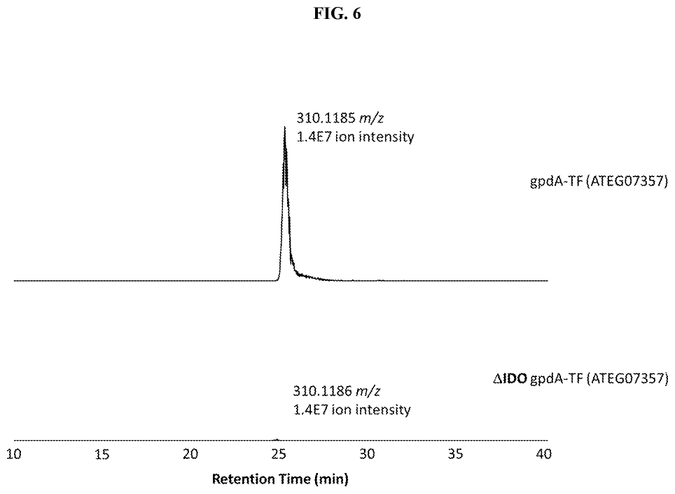

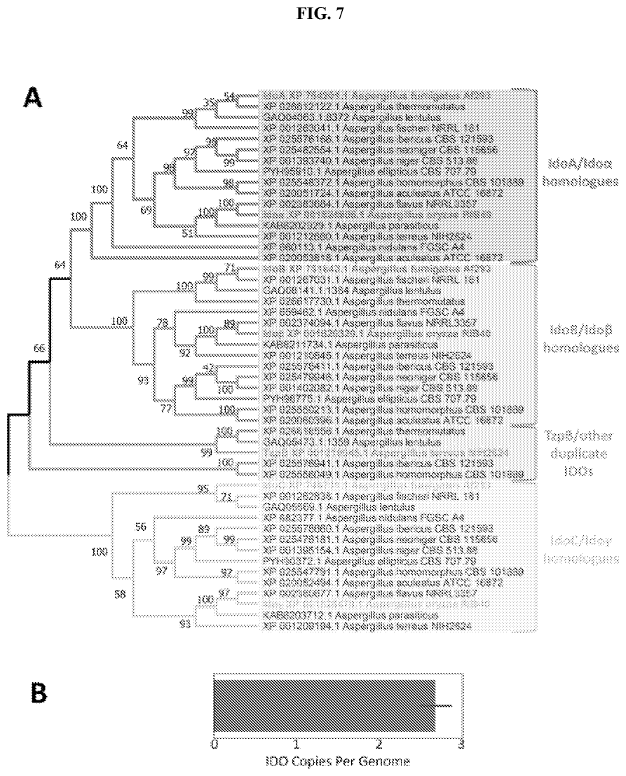

To probe terreazepine's biosynthesis, A. terreus (ATCC 20542) was grown using media containing isotopically labeled biosynthetic precursors. Labeling with ¹³C₆-anthranilate resulted in a m z shift of +6 Da (FIG. 2B), supporting incorporation of anthranilate into the molecule (FIG. 2C). Consistent with terreazepine's chemical structure, labeling with [D₅-indole]-tryptophan did not result in the expected shift of +5 in the mass spectrum, instead resulting in a mass shift of +4 (FIG. 2B). Given the existence of an IDO in the AtFAC7O19 BGC, these data provide support that tryptophan is converted into kynurenine prior to incorporation into terreazepine. For further confirmation of the IDO activity in terreazepine biosynthesis, a FAC deletion mutant was produced lacking the IDO tzpB. Mass spectral analysis of the FAC deletion mutant revealed no terreazepine production (FIG. 6).

Homology-based annotation of the FAC-encoded NRPS revealed a domain structure consisting of two adenylation (A), two condensation (C), and three thiolation (T) domains, giving the domain sequence A₁-T₁-C₁-A₂-T₂-C₂-T₃. To investigate the function of the seemingly extraneous T₃ domain, FAC truncation mutants were constructed either lacking the C₂T₃ domains (ΔC₂T₃) or only the T₃ domain (ΔT₃). These constructs were transformed into A. nidulans and extracted metabolites subjected to LC-MS analysis. A very small amount of the target compound was detected in ΔC₂T₃ extracts (5000-fold lower than control), indicating that terreazepine formation occurs slowly without catalysis. The presence of any offloaded intermediates was not detected. ΔT₃ extracts contained terreazepine levels close to that of the intact NRPS (FIG. 2D). Given that analyses focused on end-point abundance of terreazepine, it is possible that the T₃ domain increases the catalytic efficiency of product formation. This is in contrast to recent findings in which NanA, the TzpA ortholog involved in nanangelenin A biosynthesis, requires the T₃ domain for product formation (Ref. A26; incorporated by reference in its entirety).

Using heterologous expression, stable isotope feeding studies, and NRPS-backbone deletions, a biosynthetic scheme for terreazepine was determined (FIG. 2E). In this scheme, N-formyl-kynurenine is formed through the catabolism of tryptophan by TzpB, an IDO. TzpB shares 410% sequence identity to A. fumigatus IdoA and 45% identity to IdoB, and only 26% identity to IdoC. Enzymatic studies using A. oryzae IDO orthologs suggest that only Idoα and Idoβ (orthologs of IdoA and IdoB, respectively) participate in tryptophan catabolism (Refs. A27-A28). Because most Aspergilli contain three IDOs, TzpB, a fourth IDO in the parent organism Aspergillus terreus, may no longer play a role in primary metabolism and instead represent a duplicated enzyme dedicated to terreazepine biosynthesis (FIG. 7). This is reminiscent of daptomycin biosynthesis in Streptomyces roseosporus, in which the TDO DptJ supplies kynurenine for daptomycin formation (ref. A19; incorporated by reference in its entirety). The biosynthesis of terreazepine mirrors that of its relative nanangelenin A, where TzpA and TzpB orthologs in Aspergillus nanangensis (NanA and NanC) show near identical activity.

TzpA, a two-module NRPS, utilizes anthranilate and kynurenine to assemble terreazepine. The first adenylation domain (TzpA-A₁) loads anthranilate onto the T₁ domain, while TzpA-A₂ loads kynurenine, generated through spontaneous non-enzymatic deformylation of the TzpB-supplied N-formyl-kynurenine. The substrate-binding residues of TzpA-A₁ resemble those of other fungal adenylation domains which recognize anthranilate (Table 3). TzpA-A₂, responsible for incorporating kynurenine, has a new pocket code quite dissimilar from other kynurenine-binding A-domains (Table 3). However, this disparity may be attributable to evolutionarily distance between source organisms and the unstudied nature of kynurenine incorporation into fungal secondary metabolites. Given that the isolated terreazepine was a 2:1 mixture of S:R enantiomers, TzpA-A₂ may accept both (D) and (L) forms of kynurenine. The peptide bond formation between the tethered amino acids is catalyzed by the first condensation domain, TzpA-C₁, between anthranilate's carbonyl carbon and kynurenine's aliphatic primary amine. The second C domain (TzpA-C₂) catalyzes the final cyclization event between the aromatic amine of kynurenine and the tethered carbonyl carbon, yielding the final terreazepine product.

TABLE 3
Adenylation domain substrate predictions for TzpA, a nonribosomal peptide
synthetase and C2, T2, and T3 domain active site sequence alignments. (A) TzpA-A1
substrate binding residues bear similarity to many additional anthranilate-activating
adenylation domains. Additionally, adenylation domains from A. thermomutatus
(RHZ670305-A1) and A. lentulus (GAQ05471-A1) have an identical A domain sequence to
that of TzpA-A1, suggesting they also bind anthranilate. (B) TzpA-A2 possesses a specificity
sequence that is disparate from known kynurenine-binding A domains. It does, however, bear
resemblance to the A2 domains from the orphan NRPSs RHZ670305-A2, and GAQ05471-
A2, and may represent a new type of kynurenine-activating adenylation domain. (C) The C2
domain of TzpA does possess the catalytic histidine purported to be required for activity (J. A.
Baccile, H.H. Le, B.T. Pfannenstiel, J.W. Bok, C. Gomez, E. Brandenburger, D. Hoffmeister,
N.P. Keller, F.C. Schroeder, Angew Chem Int 58:14589-14593, 2019), although the
remainder of its sequence diverges from other C2 domains part of NRPSs with the
ATCATCT domain architecture such as GliP and HasD. (D) The T2 and T3 domains of
TzpA both appear functional when compared to GliP T domains and GrsA T domains with
known functionality, (G.L. Challis, J. Ravel, C.A. Townsend, Chern Biol 7:211-224, 2000)
given their sequence similarity and the presence of a conserved serine in the sequence.
Residues are colored according to the Taylor coloring scheme (W.R. Taylor. Protein
Engineering, Design, and Selection 10:743-746, 1997).
A

| NRPS | Substrate | Specificity Code | SEQ ID NO. |
|---|---|---|---|
| TA-A1 | Anthanilate | G-I-I-L-F-G-V-V-T-K | 1 |
| | (proposed) | | |
| Chrysogine synthetase | Anthranilate | G-V-I-F-M-A-A-G-V-K | 2 |
| (ADY16697) | | | |
| Benzomalvin synthetase | Anthranilate | G-I-N-F-I-G-A-G-T-K | 3 |
| (KX449366) | | | |
| Fumiquinazoline synthetase | Anthranilate | G-V-I-I-L-A-A-G-I-K | 4 |
| (EAL89049) | | | |
| Acetylaszonalenin synthetase | Anthranilate | G-A-L-F-F-A-A-G-V-K | 5 |
| (EAW16180) | | | |
| Chrysogine synthetase | Anthranilate | G-V-I-F-M-A-A-G-V-K | 6 |
| (ADY16697) | | | |
| RHZ67305-A1 | Unknown | G-I-I-L-F-G-V-V-T-K | 7 |
| GAQ05471-A1 | Unknown | G-I-I-L-F-G-V-V-T-K | 8 |

B

| NRPS | Substrate | Specificity Code | SEQ ID NO. |
|---|---|---|---|
| TzpA-A2 | Kynurenine | D-A-A-M-I-M-G-I-A-K | 9 |
| | (proposed) | | |
| nidulanin synthetase | Kynurenine | D-V-L-S-F-G-A-S-L-K | 10 |
| (CBF87869) | | | |
| Daptomycin synthetase | Kynurenine | D-A-W-T-T-T-G-V-G-K | 11 |
| (AAX31559) | | | |
| Taromycin synthetase | Kynurenine | D-A-W-T-T-T-G-V-A-K | 12 |
| (AHH53508) | | | |
| RHZ67305-A1 | Unknown | D-C-G-M-S-M-G-V-G-K | 13 |
| GAQ05471-A1 | Unknown | D-C-G-M-S-M-G-V-G-K | 14 |

C

| C₂ Doman Active Site | | | | SEQ ID NO. |
|---|---|---|---|---|
| GliP-C₂ (EAL88817) | 1753 | S-H-A-V-A-D-L-N-S | 1761 | 15 |
| HasD-C₂ (EAL92291) | 1789 | S-H-V-V-G-D-A-A-T | 1797 | 16 |
| TzpA-C₂ (EAU32742) | 2136 | T-H-A-L-W-D-G-G-P | 2144 | 17 |

D

| T Domain Ppant Binding site | | | | SEQ ID NO. |
|---|---|---|---|---|
| GrsA-T (BAA00406) | 566 | F-Y-A-L-G-G-D-S-I-K-A-I | 577 | 18 |
| GliP-T₂ (EAL88817) | 1757 | F-R-A-L-G-G-H-S-V-L-Q-M | 1586 | 19 |
| GliP-T₃ (EAL88817) | 2088 | F-F-E-A-G-G-D-S-I-Q-A-Q | 2099 | 20 |
| TzpA-T₂ (EAU32742) | 1930 | F-F-H-L-G-G-D-S-V-N-G-M | 1941 | 21 |
| TzpA-T₃ (EAU32742) | 2466 | F-F-R-L-G-G-N-S-V-R-A-L | 2477 | 22 |

While the role of the terminal TzpA-T₃ domain remains uncertain, insights are available by looking at related NRPSs. For example, the unusual NRPS domain structure of TzpA mirrors that of GliP, the NRPS involved in gliotoxin biosynthesis (Refs. A29-A30; incorporated by reference in their entireties). When studied in vitro, GliP mutants show behavior mirroring that of TzpA deletants: truncated GliP ΔT₃ mutants retain dipeptide synthetase activity, while ΔC₂T₃ mutants show reduced activity (Refs. A29-A30: incorporated by reference in their entireties). However, in vivo, GliP ΔT₃ loses activity, indicating that the in vivo pathway involves transfer of the dipeptidyl-S intermediate from T₂ to T₃ (Ref. 29; incorporated by reference in its entirety). In light of these two possible pathways of cyclization from T₂ and T₃, as well as a slow reported rate of approximately one per hour, it has been suggested that T₃ facilitates interaction with downstream tailoring enzymes (Refs. A29-A30; incorporated by reference in their entireties). Given the lack of downstream tailoring enzymes in the terreazepine pathway, both cyclization pathways may exist. Like the T domains of GliP, TzpA-T₂ and T₃ possess the predicted active site residue (S1937 and S2473, respectively), indicating that they are both functional (Table 3). Similarly, TzpA-C₂ possesses the purported catalytic histidine at position H2137. However, the adjacent residue sequence diverges from the conserved SHXXXDXXS/T (SEQ ID NO: 23) sequence shared by diketopiperazine-forming NRPSs such as GliP and HasD (29), and slightly from the SHXXXD (SEQ ID NO: 24) sequence of NanA (Ref. A26; incorporated by reference in its entirety), indicating it may have different cyclization requirements (Table 3).

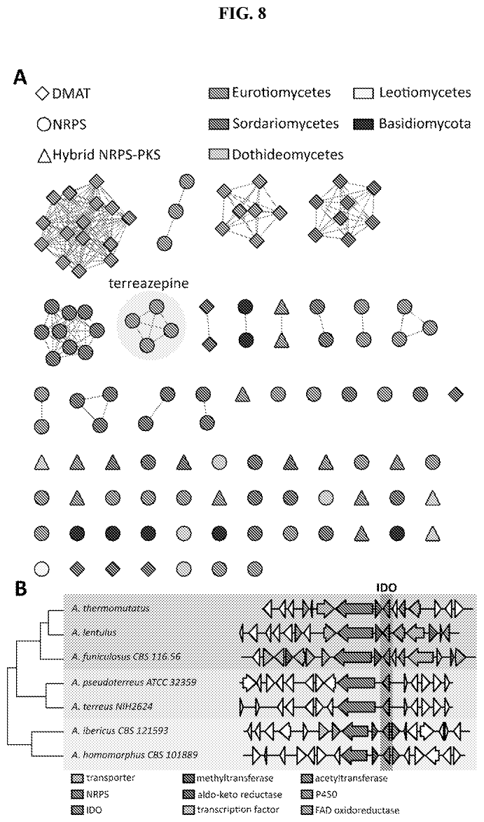

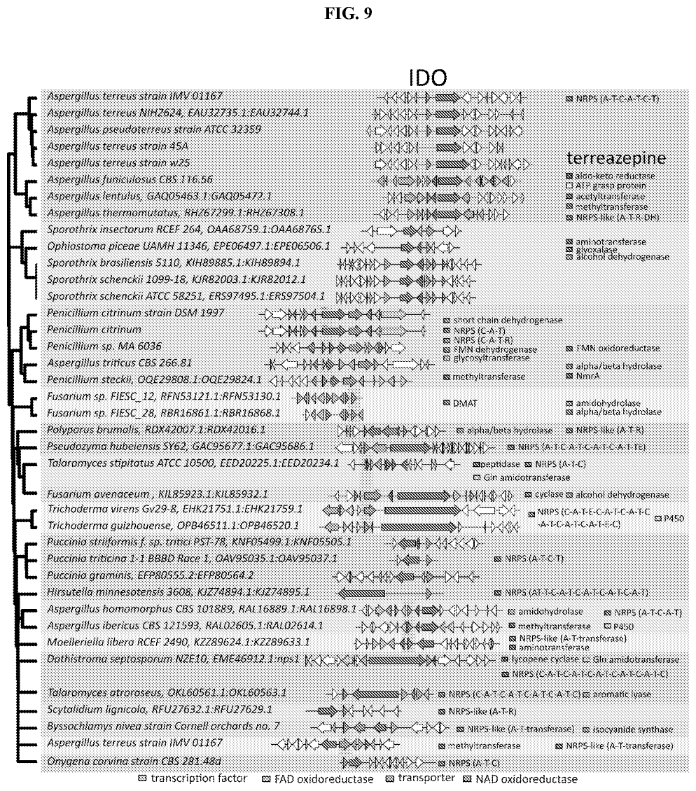

The discovery of terreazepine and its BGC revealed that fungal IDOs can play a role in secondary metabolite biosynthesis and that kynurenine incorporation into secondary metabolites can yield novel chemical scaffolds. This indicates that targeted efforts to characterize fungal BGCs containing IDOs may facilitate the discovery of completely new molecules with unique chemical scaffolds and their derivatives. Experiments were conducted during development of embodiments herein to search sequences of 1037 fungal genomes from GenBank and the Joint Genome Institute and located BGCs containing IDOs. Of the ˜38,000 BGCs contained within these genomes, 118 contain an IDO. IDO-containing BGCs were grouped into gene cluster families (GCFs) based on sequence identity and the fraction of protein domains shared between BGC pairs, anticipating that a single GCF groups BGCs that produce similar metabolites. Of the 118 IDO-containing BGCs, 68 were sorted into 16 GCFs. The remaining 50 BGCs represent singletons that had no similar BGC pairs (FIG. 8A).

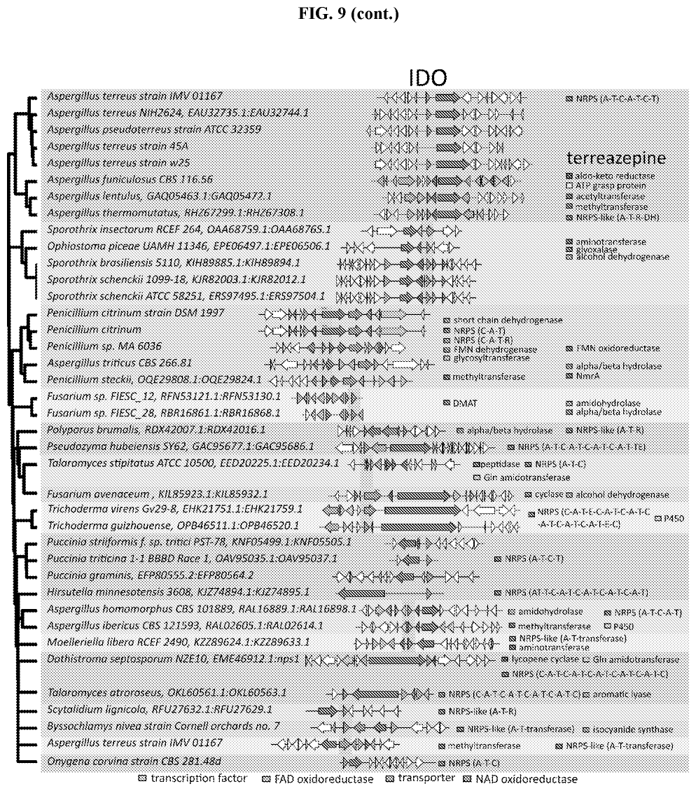

Many BGCs originate from phylogenetically diverse Aspergilli, an NRPS-containing subset of which are illustrated in FIG. 8B. BGCs from two Aspergillus GCFs in particular were identified as putative terreazepine clusters. The first GCF includes the terreazepine BGC itself, which exists in A. terreus and A. pseudoterreus. The second GCF contains BGCs from A. thermomutatus, A. funiculosus, and A. lentulus. The NRPSs in this GCF follow the same unusual domain sequence of ATCATCT (with the exception of A. lentulus which lacks the terminal T domain). Adenylation domain specificity codes bear remarkable similarity to those of TzpA-A₁ and TzpA-A₂ (Table 3), suggesting that these NRPSs biosynthesize terreazepine. Unlike the terreazepine BGC, however, the BGCs in this family contain several tailoring enzymes expected to diversify the terreazepine scaffold, raising the possibility that the shared NRPS T₃ facilitates interaction with downstream enzymes in these pathways. The tailoring enzymes present in these BGCs differ from those present in the nanangelenin A cluster in A. nanangensis, indicating that a variety of terreazepine/nanangelenin analogs may exist (Ref. A26; incorporated by reference in its entirety). Moreover, IDO-containing BGCs from A. ibericus and A. homomorphus may encode yet undiscovered dipeptide scaffolds containing kynurenine (FIG. 8B). The IDOs contained in these three GCFs represent a distinct clade of duplicated IDOs with moderate sequence homology (˜40%) to both A. fumigatus IdoA and IdoB (FIG. 7). Perhaps even more remarkable is the degree to which IDO-containing BGCs span the kingdom of fungi, encompassing five taxonomic classes and two phyla (FIG. 9). Particularly interesting is the presence of several NRPS-containing BGCs originating from Basidiomycetes, given the rare and unstudied nature of NRPSs in this phylum (Ref. A31; incorporated by reference in its entirety). Taken together, these results reveal the rich biosynthetic potential of IDO-containing BGCs that has only just begun to be explored.

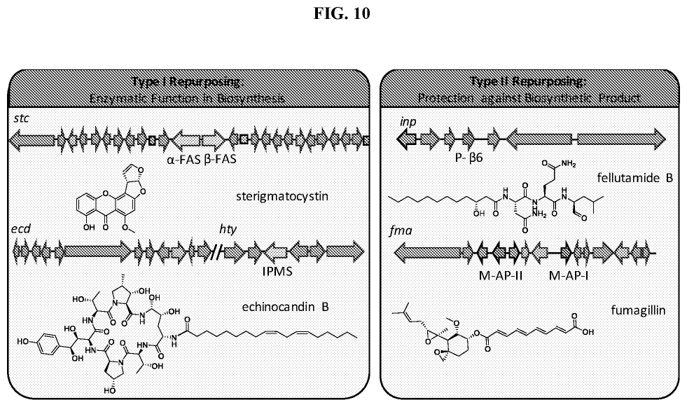

The discovery of terreazepine provides another example of how fungi repurpose primary metabolism genes for secondary metabolism. Based on this and other examples, two major strategies fungi employ for such repurposing are proposed: Type I repurposing into biosynthetic enzymes and Type II repurposing into resistance genes (FIG. 10). One of the earliest discoveries of Type I repurposing is that of the important fungal toxin sterigmatocystin. Evaluation of the sterigmatocystin biosynthetic pathway revealed the presence of two fatty acid synthase (FAS) genes, stcJ and stcK located within the sterigmatocystin gene cluster. Indeed, disruption of these genes in Aspergillus nidulans resulted in strains that did not produce sterigmatocystin, but were morphologically identical to wild-type strains (Ref A32; incorporated by reference in its entirety). Another important example of Type I repurposing is the duplicated isopropyl-malate synthase (IPMS) involved in echinocandin biosynthesis in Emericella rugulosa. Similar to the provision of kynurenine by TzpB, this duplicated IPMS serves to provide the non-proteinogenic amino acid homotyrosine for incorporation into echinocandin B (FIG. 10) (Ref. A33; incorporated by reference in its entirety).

In addition to re-purposing duplicated primary metabolism genes to have a biosynthetic role, fungi also utilize duplicated genes from primary metabolism as a form of self-resistance (Refs. A34, A35; incorporated by reference in their entireties). This Type II repurposing represents a particularly attractive avenue for drug discovery, as the duplicated gene will often provide insight into the mechanism of action of the encoded secondary metabolite. Several examples of such Type II repurposing have been discovered by targeting clusters with duplicate resistance targets. The proteasome inhibitor fellutamide B, for example, was discovered due to the presence of a duplicated proteasome subunit within its BGC (36). Similarly, the BGC encoding the methionine aminopeptidase inhibitor fumagillin contains both type I and type II methionine aminopeptidase genes in the gene cluster (FIG. 10) (Ref. A37; incorporated by reference in its entirety). While it is likely that many of the IDOs contained within the BGCs depicted in FIGS. 8 and 9 represent Type I biosynthetic enzymes that provide kynurenine for secondary metabolite synthesis, it is also possible that they represent Type II duplicated gene targets that serve to protect the producing organism against the biosynthetic product. Indeed, It was contemplated that terreazepine might possess IDO inhibitory activity and show promise as an anti-cancer agent (Ref. A38; incorporated by reference in its entirety). When tested against A. fumigatus IDO mutants, however, no growth inhibitory activity was observed. Studies aimed to elucidate the biosynthetic products of additional IDO-containing BGCs in fungi offer exciting opportunities not only to discover new molecular scaffolds, but to identify anti-cancer metabolites with known mechanisms of action.

### Example 2

### Interpreted Atlas of Biosynthetic Gene Clusters from Fungal Genomes

The concept of a gene cluster family (GCF) has emerged as an approach for large-scale analysis of BGCs (Ref. B5-B8; incorporated by reference in their entireties). The GCF approach involves comparing BGCs using a series of pairwise distance metrics, then creating families of BGCs by setting an appropriate similarity threshold. This results in a network structure that dramatically reduces the complexity of BGC datasets and enables automated annotation based on experimentally characterized reference BGCs. Depending on the similarity threshold, BGCs within a family are expected to encode identical or similar metabolites and therefore serve as an indicator of new chemical scaffolds. The use of GCFs represents a logical shift from a focus on single genomes of interest to large genomics datasets, providing a means of regularizing collections of BGCs and their encoded chemical space (Fig. B1A). The use of GCF networks has been utilized for global analyses of bacterial biosynthetic space (Ref. B6; incorporated by reference in its entirety), bacterial genome mining at the >10,000 genome scale (Refs. B9, B16; incorporated by reference in their entireties), and integrated with metabolomics datasets for large-scale compound and BGC discovery (Refs. B5, B7; incorporated by reference in their entireties). Together with advances to large-scale metabolomics data analysis such as molecular networking (Ref. B17; incorporated by reference in its entirety), the GCF paradigm has helped in the modernization of natural products discovery.

Application of GCFs to fungal genomes has been limited to datasets of <100 genomes from well-studied genera such as Aspergillus, Fusarium, and Penicillium (Refs. B13-B15). Despite the availability of thousands of genomes representing a broad sampling of the fungal kingdom, global analyses of the BGC content of these genomes are lacking. As such, knowledge of the overall phylogenetic distribution of GCFs in fungi is limited, and many taxonomic groups have no experimentally characterized BGCs. Experiments were conducted during development of embodiments herein to perform a global analysis of BGCs and their families from a dataset of 1037 genomes from across the fungal kingdom. Across Fungi, the vast majority of GCFs are species-specific, indicating that species-level sampling for genome sequencing and metabolomics will yield significant returns for natural products discovery.

To relate this now-available set of fungal GCF-encoded metabolites to known fungal scaffolds, network analysis of 15,213 fungal compounds was conducted during development of embodiments herein, organizing these into 2,945 molecular families (MFs) (Fig. B1A). Analysis of this joint genomic-chemical space revealed dramatic differences between both major fungal taxonomic groups, as well as between bacteria versus fungi, thus laying the groundwork for systematic discovery of new compounds and their BGCs from the fungal kingdom.

### A Reference Set of Fungal Biosynthetic Gene Clusters

Despite the availability of thousands of fungal genomes, the biosynthetic space represented within them has not been surveyed systematically, prior to the work described herein. To address this gap, a dataset of 1037 fungal genomes was curated, covering a broad phylogenetic swath (Table 4). This selection includes well-studied taxonomic groups such as Eurotiomycetes (Aspergillus and Penicillium genera) and Sordariomycetes (Fusarium, Cordyceps, and Beauveria genera), and groups for which little is known regarding their BGCs, such as Basidiomycota or Mucoromycota. This genomic sampling covers a large swath of ecological niches, from forest-dwelling mushrooms to plant endophytes to extremophiles (Ref. B18; incorporated by reference in its entirety).

TABLE 4
Genomes analyzed in this study and the distribution of their gene clusters classified by biosynthetic type.

| | | | | | | | | | Per- |
|---|---|---|---|---|---|---|---|---|---|
| | | | | | | | | Genomes | genome |
| Taxon | NRPS | HYBRID | HRPKS | TERPENE | NRPSLIKE | NRPKS | DMAT | analyzed | average |
| Pucciniomycotina | 2.0 | 0.0 | 0.0 | 0.6 | 0.6 | 0.0 | 0.0 | 25 | 3.2 |
| Ustilaginomycotina | 3.0 | 0.6 | 0.1 | 0.0 | 3.6 | 6.9 | 0.3 | 32 | 6.5 |
| Agaricomycotina | 1.6 | 0.6 | 0.1 | 6.1 | 4.5 | 1.0 | 0.2 | 173 | 14.1 |
| Pezizomycotina | 9.0 | 5.4 | 4.7 | 4.5 | 7.9 | 7.1 | 1.2 | 721 | 39.8 |
| Taphrinomycotina | 1.2 | 0.0 | 0.0 | 0.5 | 1.1 | 0.0 | 0.0 | 12 | 2.8 |
| Mucorosomycota | 1.1 | 0.2 | 0.0 | 1.8 | 2.6 | 0.0 | 0.0 | 36 | 5.5 |
| Zoopagomycota | 3.8 | 0.1 | 0.1 | 0.3 | 0.7 | 0.1 | 0.0 | 16 | 5.1 |
| Blastocladicmycota | 1.5 | 0.0 | 0.0 | 0.0 | 0.5 | 0.0 | 0.0 | 2 | 2 |
| Chytridiomycota | 9.1 | 0.9 | 0.8 | 0.1 | 1.6 | 1.2 | 0.0 | 12 | 13.7 |
| Microsporidia | 0.0 | 0.0 | 0.0 | 0.0 | 0.0 | 0.0 | 0.0 | 1 | 0 |
| Cryptomycota | 0.0 | 0.0 | 0.0 | 0.0 | 0.0 | 1.0 | 0.0 | 1 | 1.0 |

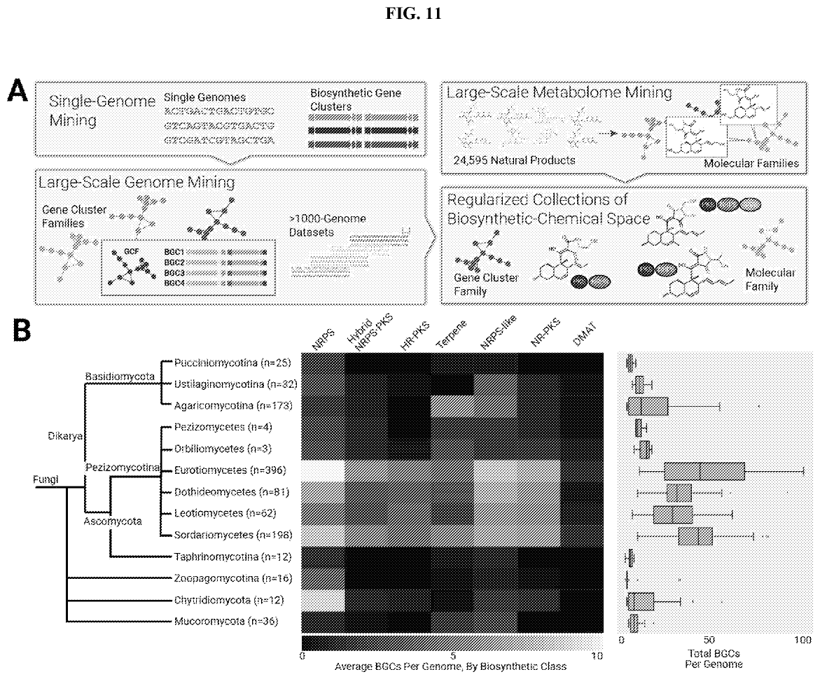

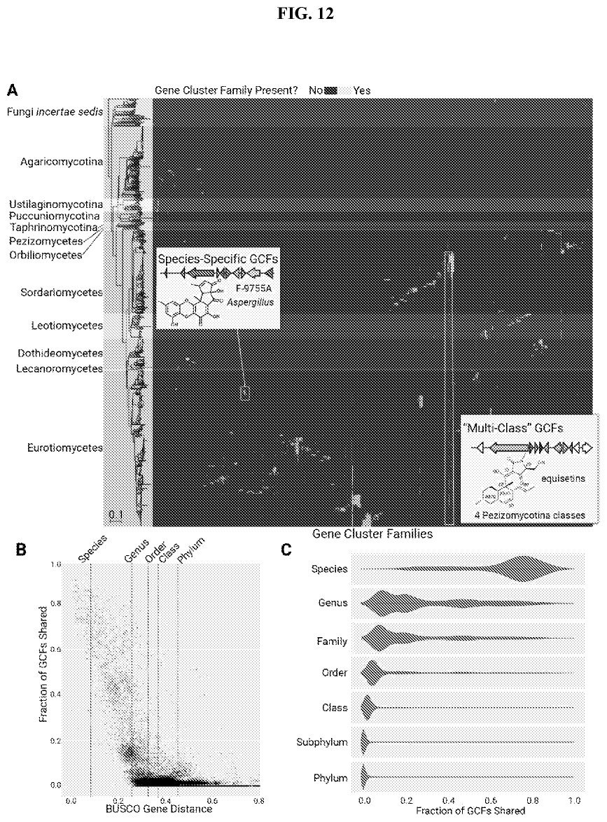

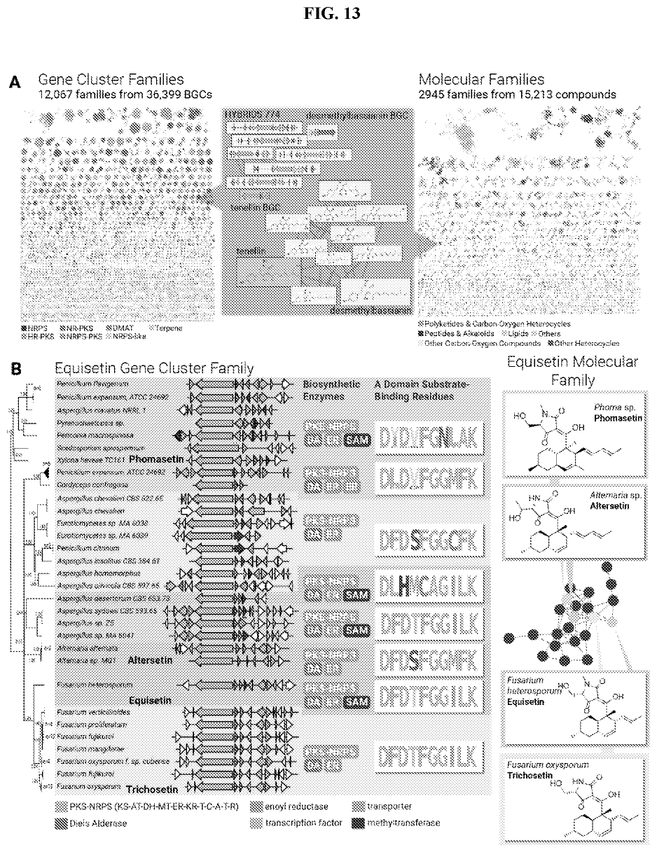

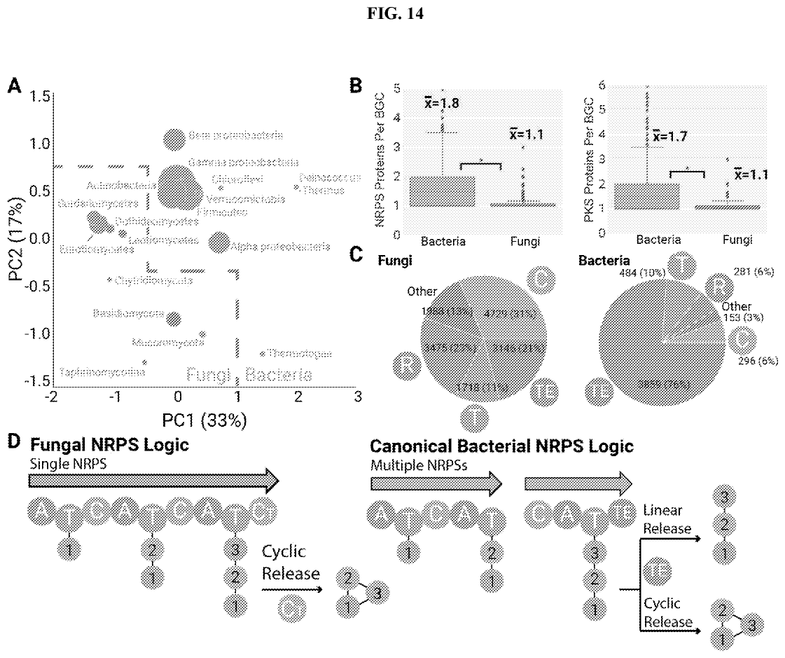

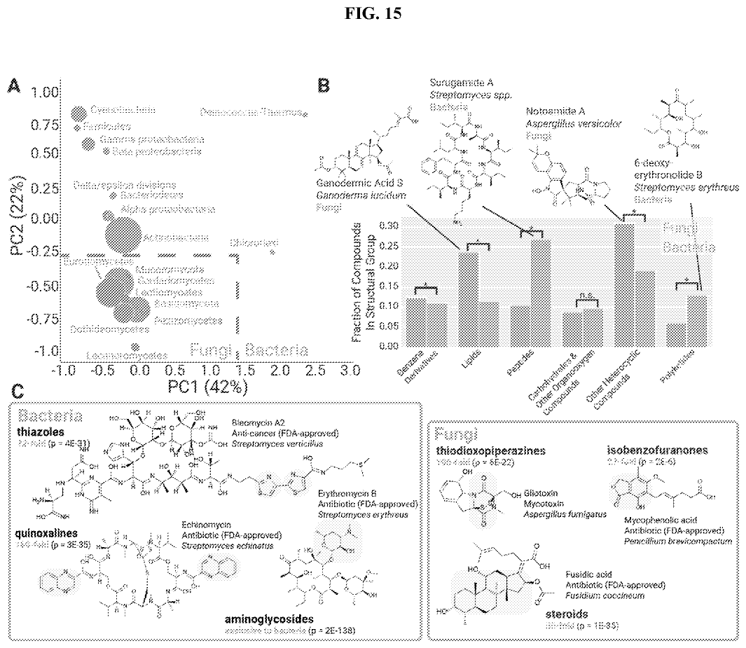

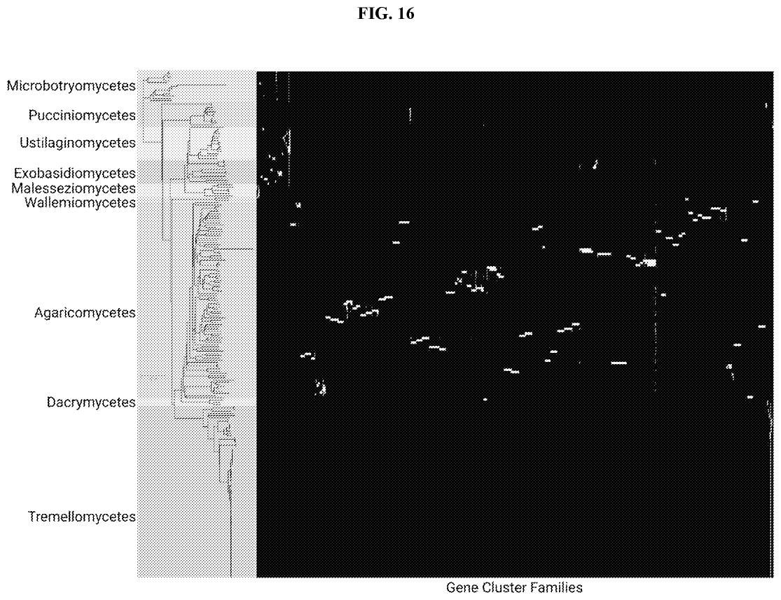

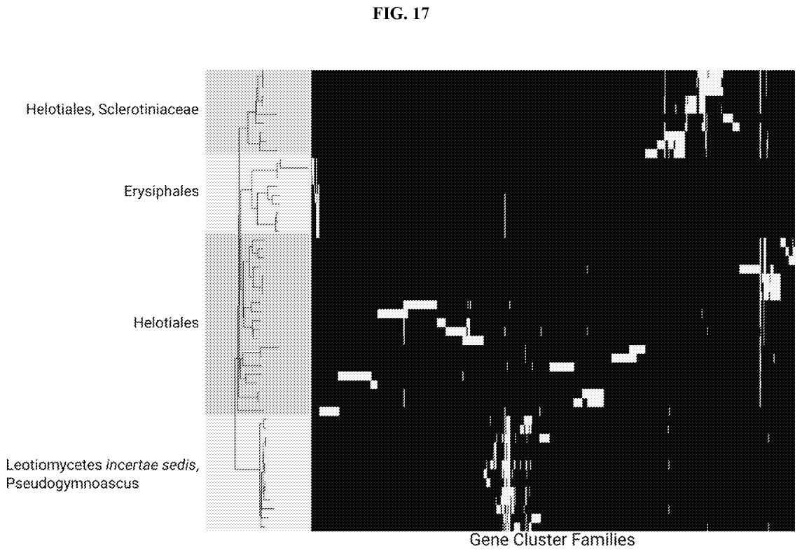

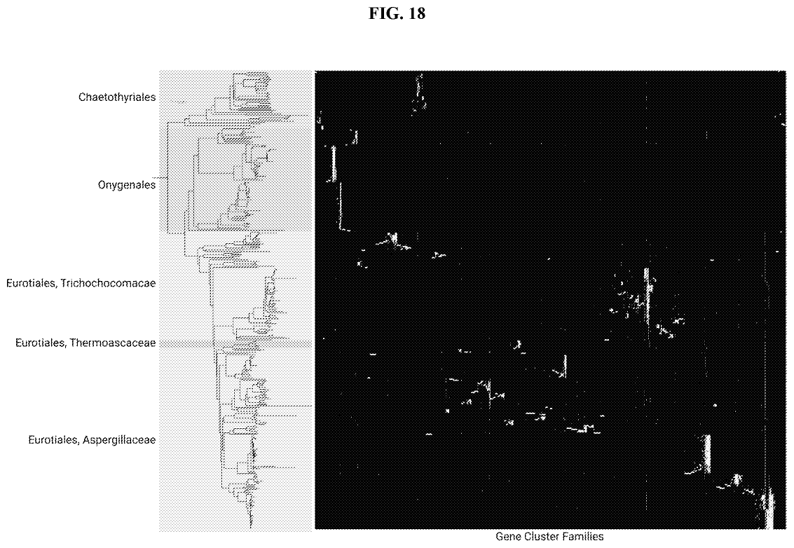

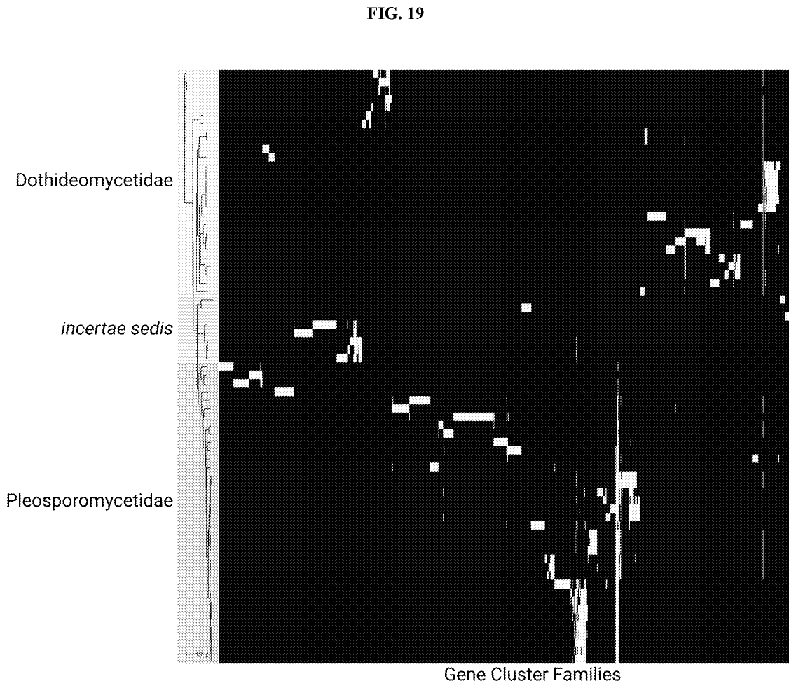

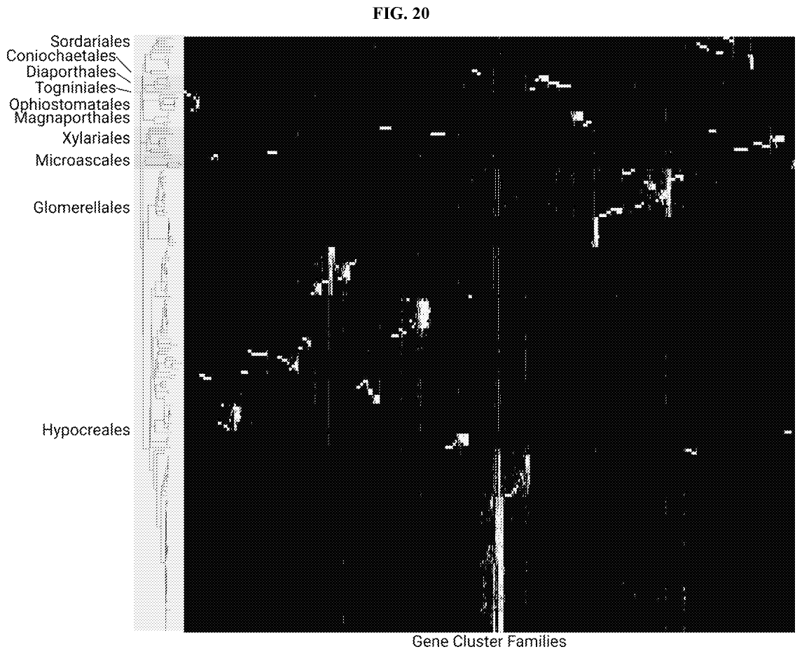

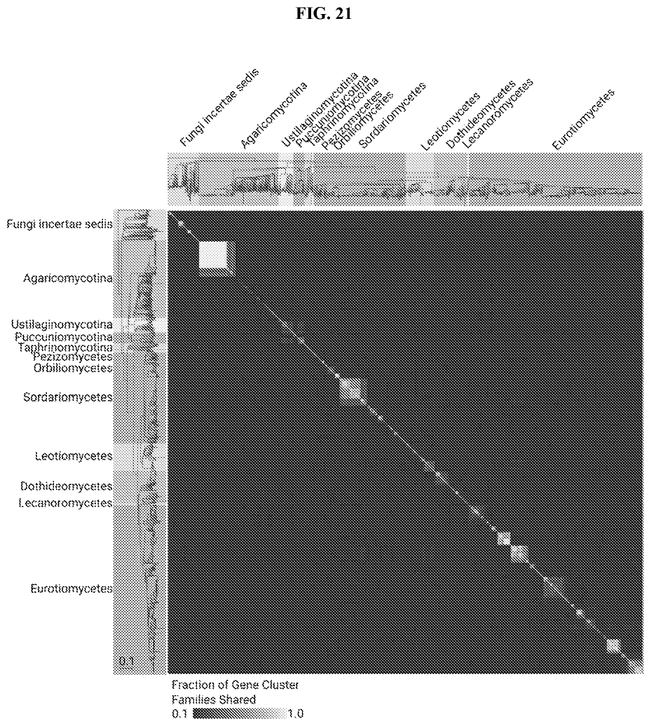

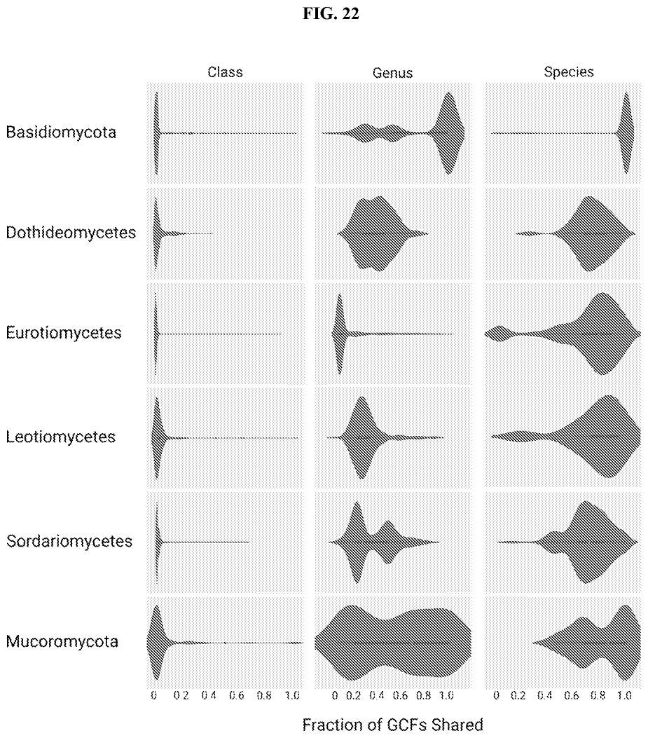

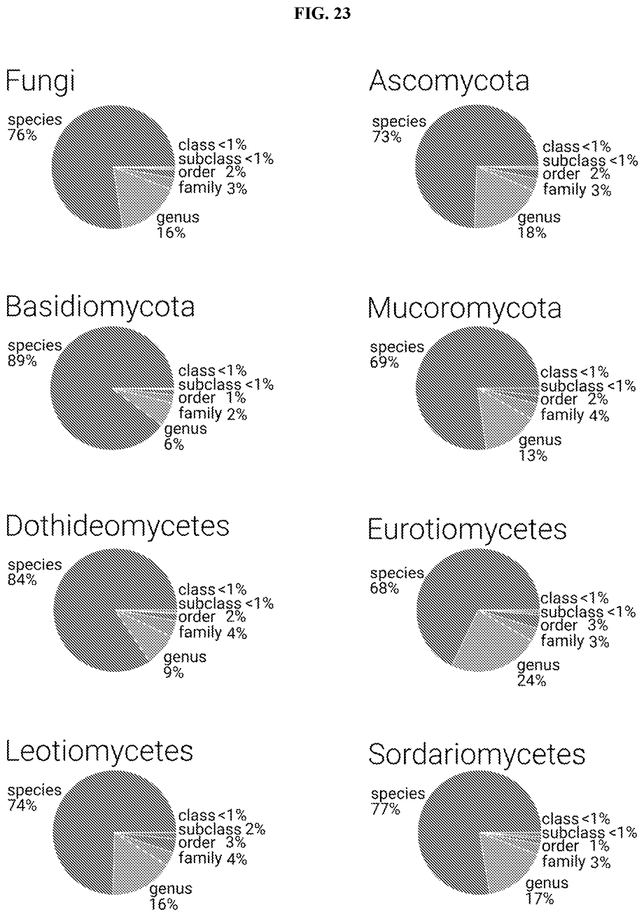

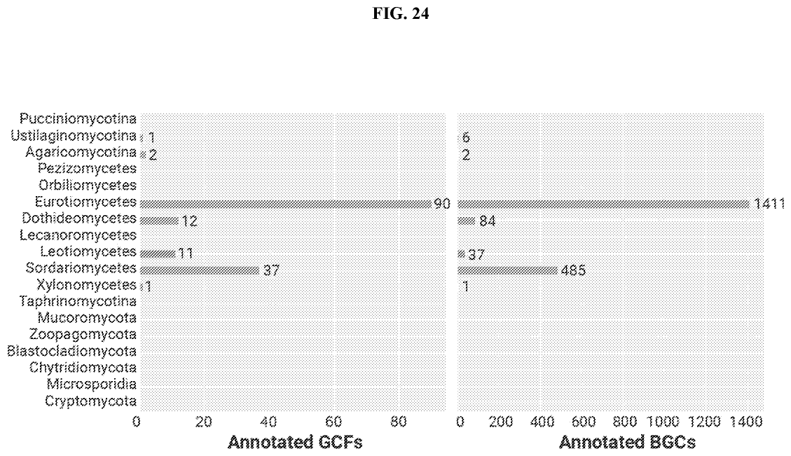

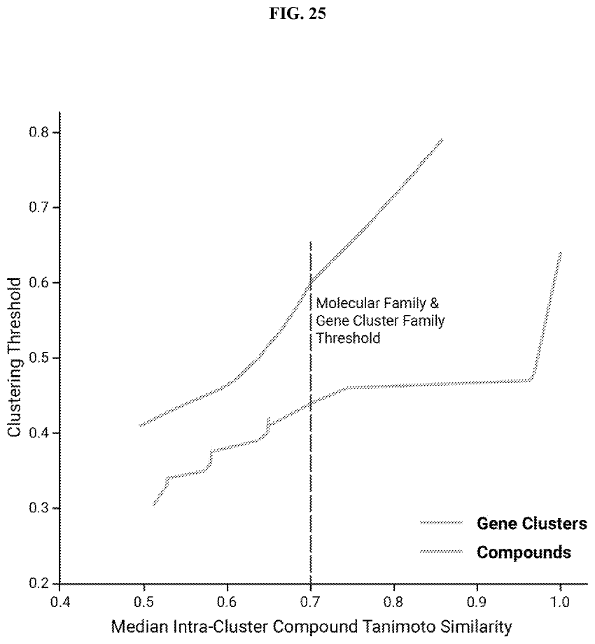

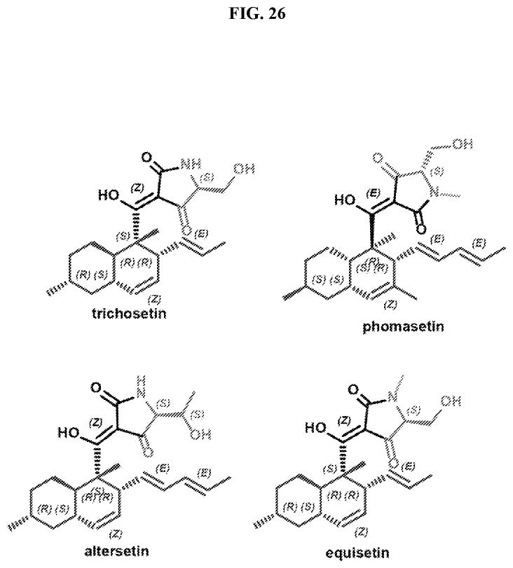

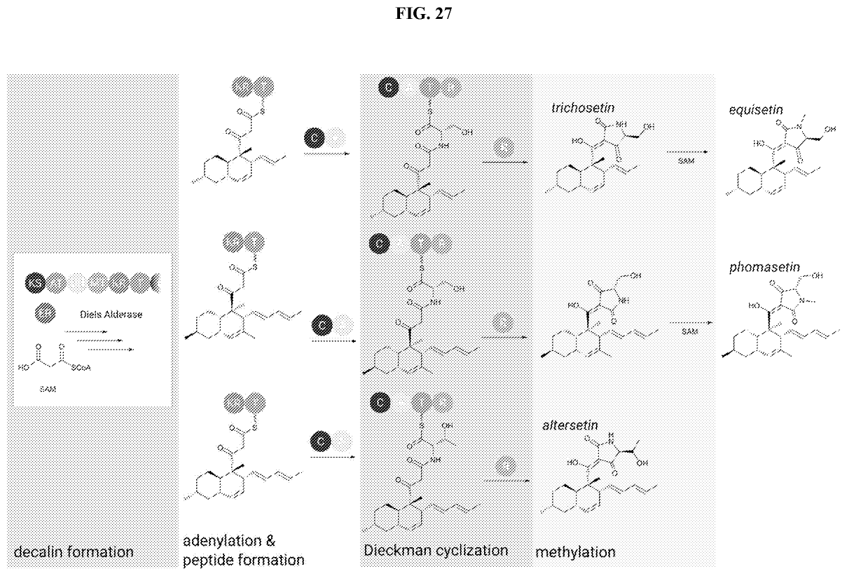

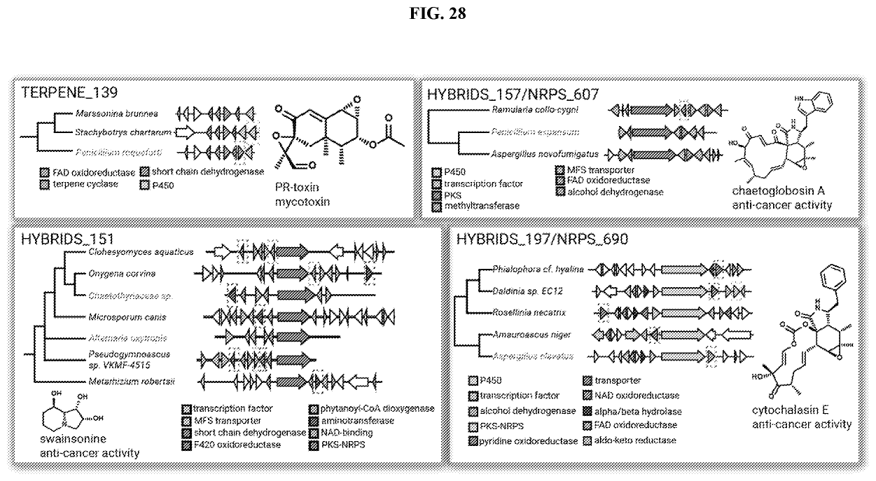

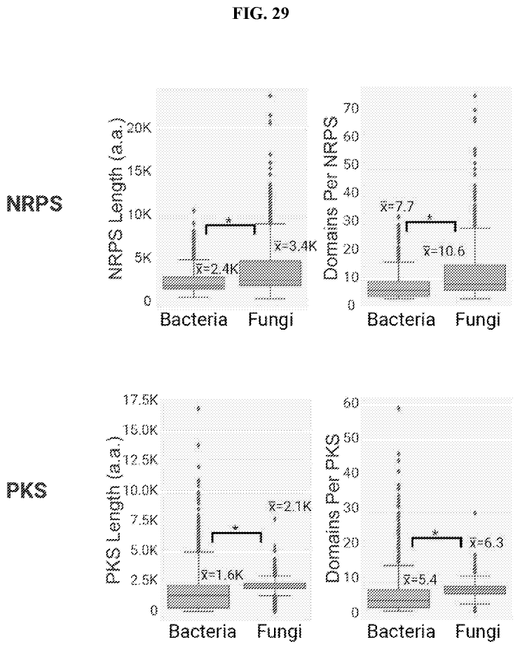

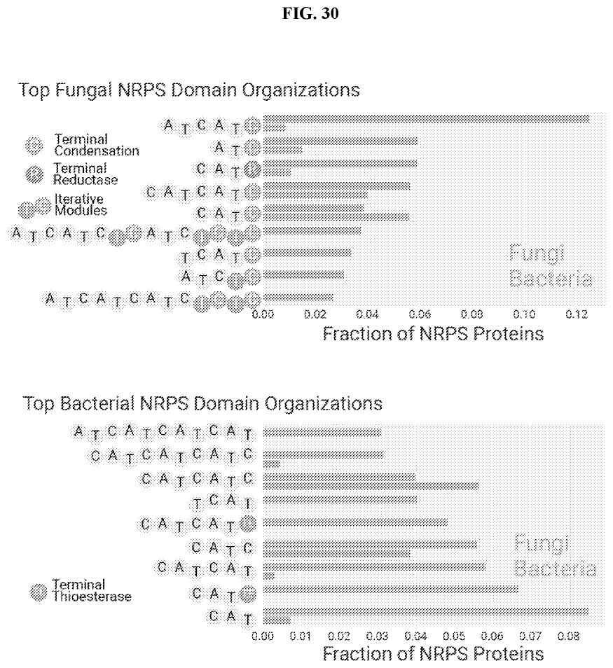

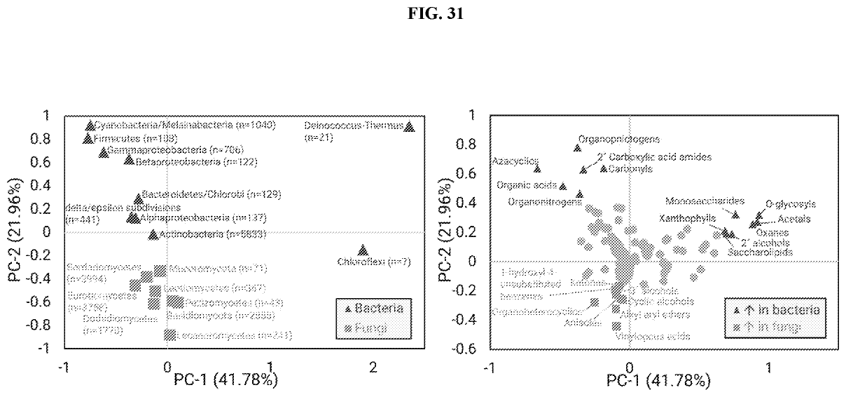

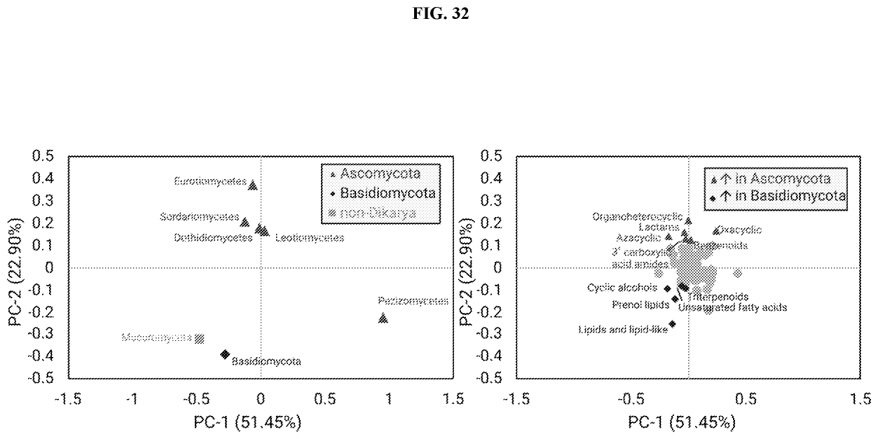

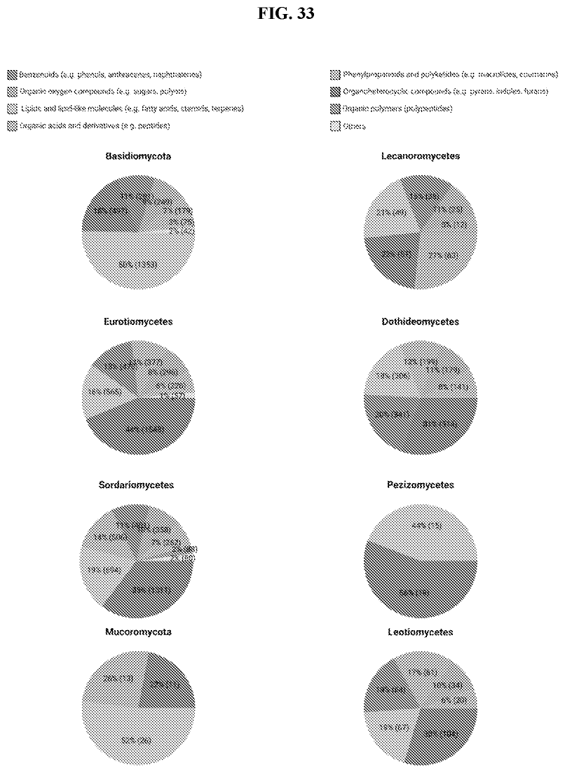

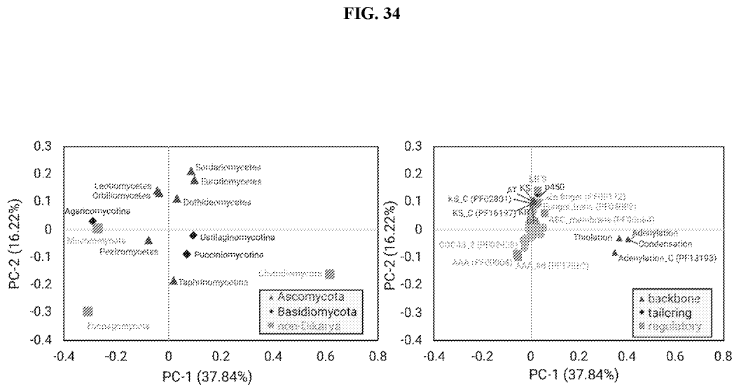

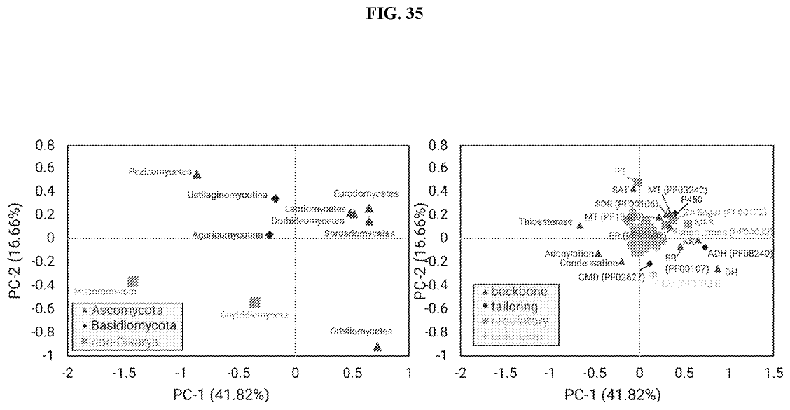

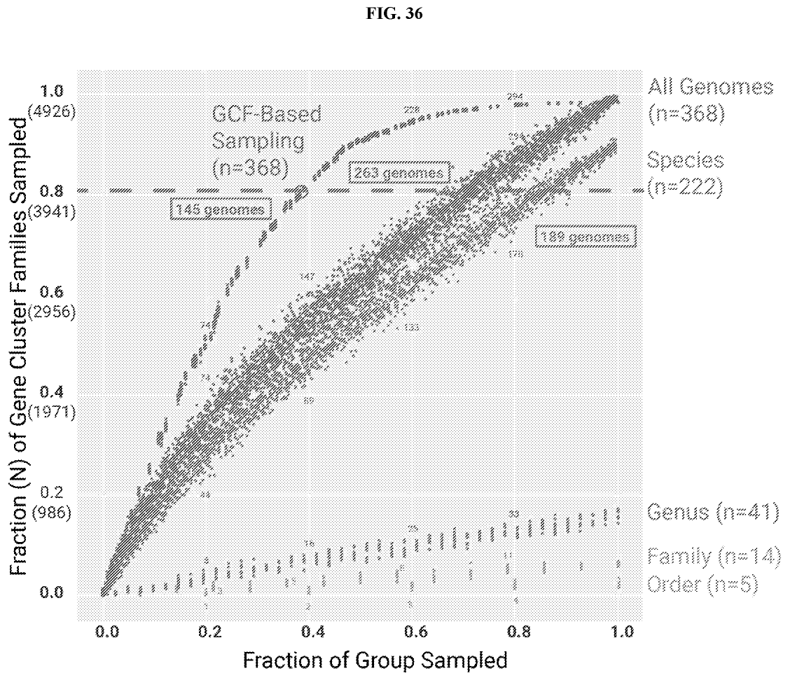

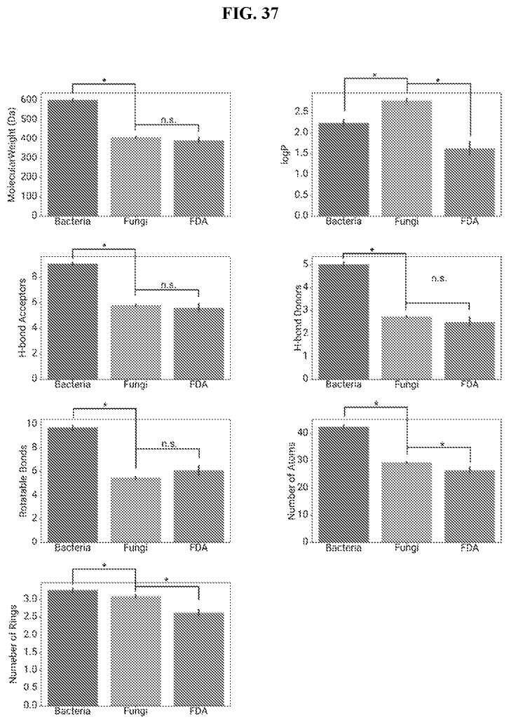

Each of the 1037 genomes was analyzed using antiSMASH (Ref 19; incorporated by reference in its entirety), yielding an output of 36,399 BGCs ranging from 5 to 220 kb in length. As has been previously observed (Ref 20; incorporated by reference in its entirety), the number of BGCs per genome varies dramatically across Fungi (FIG. 11; Table 4). Eurotiomycetes average 48 BGCs per genome, with 25% of organisms within this class possessing >60 BGCs. Organisms outside of Pezizomycotina possess significantly fewer BGCs, with organisms from the non-Dikarya phyla averaging <15 BGCs per genome. The distribution of biosynthetic classes across the fungal kingdom also varies dramatically and unexpectedly. Organisms within the Pezizomycotina classes Eurotiomycetes, Dothideomycetes, Leotiomycetes, and Sordariomycetes average approximately 5 each of NRPS, hybrid NRPS-PKS, NRPS, HR-PKS, terpene, NRPS-like, and NR-PKS, and 2 DMAT BGCs per genome (see FIG. 11B). Basidiomycota have far fewer BGCs encoding a relatively limited chemical repertoire, with terpene BGCs being the most abundant in Agaricomycotina, as previously implied (Ref. B10; incorporated by reference in its entirety).

Organizing Gene Clusters into Families to Map Fungal Biosynthetic Potential

To further assess the ability of fungi to produce new chemical scaffolds, BGCs were grouped into families using the pairwise distance between BGCs and a clustering algorithm to yield GCFs. BGCs from antiSMASH were converted to arrays of protein domains then compared based on the fraction of shared domains and backbone protein domain sequence identity (Refs. B7, B8; incorporated by reference in their entireties). DBSCAN clustering was performed on the resulting distance matrix, resulting in a set of 12,067 GCFs (Fig. B2A) organized into a network (Fig. B3A). Across the fungal kingdom, the distribution of GCFs shows a clear relationship with phylogeny (see yellow streaks in Fig. B2A, Figs. BS1-BS5). In isolated studies of well-characterized strain sets of Aspergillus and Penicillium, GCFs have been thought to be largely genus- or species-specific (Refs. B13, B21, B22); however, here we show that several GCFs span entire subphyla or classes (Fig. B2A). The fraction of GCFs that two organisms share is likewise correlated with phylogenetic distance, evidenced by sets of shared GCFs between closely related taxonomic groups (Fig. BS6; IBG). In order to facilitate visualization of these phylogenetic patterns, a web-based application was developed for hierarchical browsing of GCFs, BGCs, protein domains and annotations for known compound/BGC pairs (http://prospect-fungi.com). Additional details of the site are available in SI Methods.

Experiments were conducted during development of embodiments herein to quantify the relationship between phylogeny and shared GCF content. The protein sequence identity of 290 shared single-copy orthologous genes from the fungal BUSCO dataset (Ref. B23; incorporated by reference in its entirety) was used as a proxy for whole-genome distance. The fraction of GCFs shared within each genome was counted in pairwise comparisons (Fig. B2B). A result was a clear relationship between genomic distance and shared GCF content, with an average of 75% shared GCFs at the species level, but less than 5% shared GCFs at taxonomic ranks higher than family (FIG. 2C). A similar trend exists for individual phyla and taxonomic classes (Fig. BS7). Across the fungal kingdom, 76% of GCFs are species-specific and only 16% are genus-specific (Fig. BS8), indicating that most BGCs enable fungi related at the species level to secure their respective ecological niches with highly specialized compounds (Ref. B4; incorporated by reference in its entirety).

### GCF-Enabled Annotation of Fungal Biosynthetic Repertoire Anchored by Known BGCs

Identifying BGCs that have known metabolite products is an important component of genome mining, enabling researchers to prioritize known versus unknown biosynthetic pathways for discovery. These “genomic dereplication” efforts have been bolstered by the development of the MIBiG repository (Ref. B24; incorporated by reference in its entirety), which contained 213 fungal BGCs with known metabolites, as of June 2019. When anchored with known BGCs, the GCF approach enables large-scale annotation of unstudied BGCs based on similarity to reference BGCs, identifying clusters likely to produce known metabolites or derivatives of knowns.

Within the dataset, 154 GCFs contained known BGCs from MIBiG, approximately 1% of the 12,067 total GCFs reported here (Fig. BS9). These families collectively include a total of 2,026 BGCs (Fig. BS9), an approximately 10-fold increase in the number of annotated BGCs over that available in MIBiG (Ref. B24; incorporated by reference in its entirety). This expanded set of annotated BGCs and their families was made available for routine genome mining via the web.

### Large-Scale Comparison of GCFs and Fungal Compounds

To assess the relationship between GCFs and their chemical repertoire, GCF-encoded scaffolds were compared to a dataset of known fungal scaffolds. Analogous to the GCF analysis, network analysis of fungal metabolites was utilized, organizing these compounds into molecular families (MFs) based on Tanimoto similarity, a commonly used metric for determining chemical relatedness (Refs. B25, B26; incorporated by reference in their entireties). To directly relate GCF and MF-encoded metabolite scaffolds, the relationship between chemical similarity and BGC similarity was determined for a set of 154 fungal GCFs with known metabolite products (Fig. BS10). An MF similarity threshold was selected that resulted in similar levels of chemical similarity represented by GCF and MF metabolite scaffolds.

Using this compound network analysis strategy, a dataset of 15,213 fungal metabolites from the Natural Products Atlas (Ref. B27; incorporated by reference in its entirety) was organized into 2,945 MFs (Fig. B3A). Each compound was annotated within this network with chemical ontology information using ClassyFire, a tool for classifying compounds into a hierarchy of terms associated with structural groups, chemical moieties, and functional groups (Table 5) (Ref. B28; incorporated by reference in its entirety). The number of MF scaffolds (2,945) is only 25% the number of GCF-encoded scaffolds (12,067) in the 1000-genome dataset. This indicates that even this small genomic sampling of the entire fungal kingdom, estimated to have >1 million species (Ref. B29; incorporated by reference in its entirety), possesses biosynthetic potential that significantly dwarfs know fungal chemical space—not only in terms of individual metabolites, but also in terms of metabolite scaffolds. In this joint GCF-MF dataset, molecular families and gene cluster families represent complementary approaches for representing the same metabolite scaffold, such as the tenellin/desmethylbassianin structural class, whose GCF and MF contains both BGCs and compounds, respectively (Fig. B3A, middle).

TABLE 5
Chemical ontology-based classification of metabolites from Aspergillus fumigatus. Each chemical ontology entry in the table contains
the major ontology superclass in bold, followed by other chemical ontology terms.

| Metabolite Name | Structure | Chemical Ontology Terms |
|---|---|---|
| 1,2-dihydro-16-O- [symbol] acid 21.18-actone | 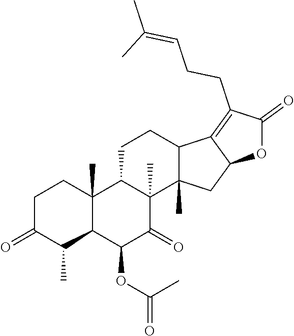 | Lipids and lipid-like molecules. Steroids and steroid derivatieves. Steroid lactones. Steroid esters, 7- [symbol] , 3-cis-6-alph-steroids, [symbol] acids and derivatives, [symbol] [symbol] Oxacyclic compounds, Organic oxides, Hydrocarbon derivatives |
| 11-methyl-11- [symbol] acid amide | 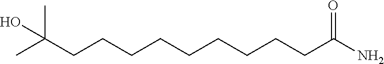 [SMILES: CC(C)(O)CCCCCCCCCC(N)=O] | Organic oxygen compounds. Organoxygen compound, Alcohols and polyoids, Tertiary alcohols, Carboximodic acids, Organonitrogen compounds, Organonitrogen compounds, Hydrocarbon derivatives |
| 11-O- [symbol] A | 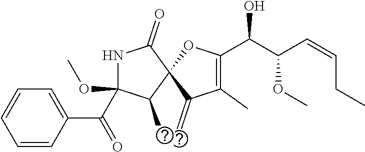 [SMILES: C=C1C(C)=C([C@@H](O)[C@H](/C=C\CC)OC)O[C@]12C(=O)N[C@@](OC)(C(=O)c1ccccc1)[C@@H]2C] | Organic oxygen compounds. Organooxygens compounds, Carbonyl compounds, Ketones, Aryl ketones, Phenylketones, Alkyl-phenyketones, Benroyl derivatives, Aryl alkyl ketones, Pyrrolidine-2-ones, [symbol] Vinylogous esters, Secondary carboxylic acid amides, Secondary alcohols, Lactams, Oxacyclic compounds, Dialkyl ethers, Azacyclic compounds, Organonitrogen compounds, Organic oxides, Hydrocarbon derivatives |
| 13- [symbol] | 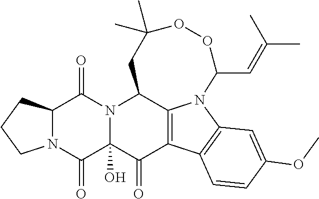 | Organoheterocyclic compound. Indoles and derivatives. [symbol] Alpha amino acids and derivatives, Indoles, 2,5- [symbol] , Aryl alkyl ketones, Anisoles, N-alkylpiperazines, Alkyl aryl ethers, Vinylogous amides, Tertiary carboxylic acid amides, Pyrroldines, [symbol] , Heteroaromatic compounds, Lactams, Dialkyl peroxides, Oxacyclic compound, Azacyclic compounds, [symbol] Hydrocarbon derivatives |
| 2,4,6,8- [symbol] | 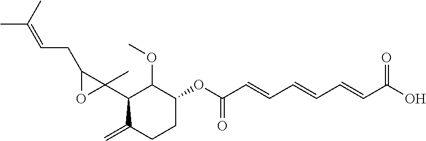 | Lipids and lipid-like molecules. Fattty Acylis, Fatty acids and [symbol] Medium-chain fatty acids, Fatty acids esters, Epoxy fatty acids, [symbol] fatty acid, Unsaturated fatty acids, Dicarboxylic acids and derivatives, [symbol] esters, Oxacyclic compounds, Epoxides, Dialkyl esters, Carboxylic acids, Organic nodes, Hydrocarbon derivatives, Carbonyl compounds. |
| 2-chloro-1,2,8-trihydroxy-6- methylanthone | 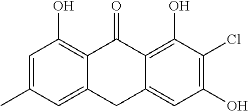 [SMILES: Cc1cc(O)c2c(c1)Cc1cc(O)c(Cl)c(O)c1C2=O] | Benzenoids. [symbol] , Aryl ketones, 1-hydroxyl-4-unsubstituted benzenoids, 1-hydroxy-2-unsubstituted benzenoids, Aryl chlorides, Vinylogous acids, [symbol] [symbol] , Organic oxides, Hydrocarbon derivatives |
| 3-hydroxy-2,5-toloquinone | 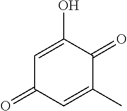 | Organic oxygen compound. [symbol] compounds, Carbonyl compounds, Ketones, Cyclic ketones, Quinones, Benzeoquinones, P-benzoquinones, Vinylogous acids, [symbol] , Organic oxides, Hydrocarbon derivatives |
| [symbol] | 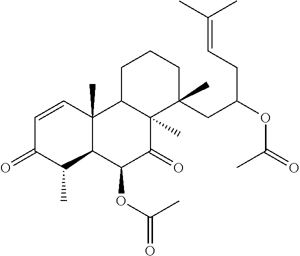 [SMILES: CC(=O)OC(CC=C(C)C)C[C@@]1(C)CCCC2[C@]1(C)C(=O)[C@@H](OC(C)=O)[C@H]1[C@H](C)C(=O)C=C[C@]21C] | Benzenoids. [symbol] and derivatives, [symbol] Alpha-acyloxy ketones, Dicarboxlic acids and derivatives, Carboxylic acid esters, Organic oxides, Hydrocarbon derivatives |
| S-N-acelylardeemin | 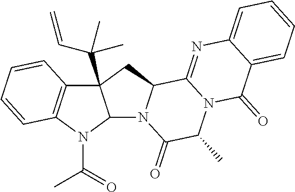 | Organoheterocyclic compounds. Indoles and derivatives, Pyrrolasdicles, Quinazolnes, Indoles, Pysimidones, Benzenoids, [symbol] Heteraromatic compounds, Pyrrolidine, Pyrroles, Lactams, Azacyclic compounds. Carbonyl compounds, Hydrocarbon derivatives, Organic oxides, Organonitrogen compounds, Organopnitrogen compounds |
| 6- [symbol] | 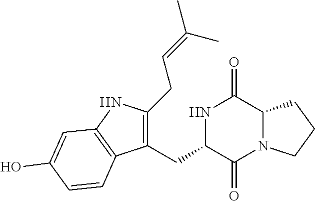 [SMILES: CC(C)=CCc1[nH]c2cc(O)ccc2c1C[C@@H]1NC(=O)[C@@H]2CCCN2C1=O] | Organic acids and derivatives. Carboxylic acids and derivatives. Amino acids, peptides, and analogues. Amino acids and derivatives, Alpha amino acids and derivatives, 3-alkylindoles, Hydroxyindoles, 2,5-diaxopiperazines, [symbol] -hydroxy-2-unsubstituted benzenoids, N-akylpiperazines. Substituted pyrroles, Tertiary carboxylic acid amides, Pyrrolidines, Heteroaromatic compounds. Secondary carboxylic acid amides, Lactams. Azacyclic compounds. Carbonyl compounds. Hydrocarbon derivatives, Organic oxides, Organonitrogen compounds, Organopnitrogen compounds |
| Asperfumigatin | 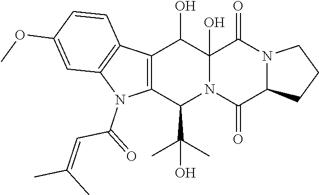 [SMILES: COc1ccc2c3c(n(C(=O)C=C(C)C)c2c1)[C@H](C(C)(C)O)N1C(=O)[C@@H]2CCCN2C(=O)C1(O)C3O] | Organoheterocyclic compounds. Iodotes and derivatives, [symbol] Alpha amino acids and derivatives, 3-alkylindoles, Anisoles, 2,5-dioxolperazines, N-alkylpiperazines, Alkyl aryl ethers, Substutites pyrroloes, Tertiary carboxylic acid amides, Tertiary alcohols, Pyrrolidines, Hetercaromatic compounds, Secondary alcohols, Lactams, Azacyclic compounds, [symbol] compounds, Organic oxides, Hydrocarbon derivatives, Carbonyl compounds |
| Cephasimysin A | 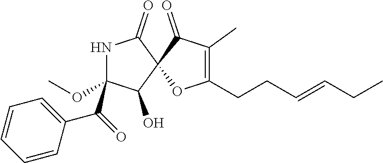 [SMILES: CC/C=C/CCC1=C(C)C(=O)[C@]2(O1)C(=O)N[C@](OC)(C(=O)c1ccccc1)[C@@H]2O] | Organic oxygen compounds. Organooxygen compounds, Carbonyl compounds, Ketones, Aryl ketones, [symbol] , Aryl alkyl ketones, Pyrrolidine-2-ones, Furanones, Vinylogous ester, Secondary carboxylic acid amides, Secondary alcohols, Lactams, Oxacyclic compounds, Azacyclic compounds, Organonitrogen compounds, Organic oxides, Hydrocarbon derivatives |
| [symbol] | 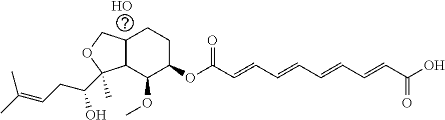 | Organoheterocyclic compounds. Isobenzenfurans, Medium-chain fatty acides, Branched fatty acids, Hydroxy fatty acids, Hetercyclic fatty acids, Fatty acid esters, Unsaturated fatty acids Dicarboxylic acids and derivatives, Tetrahydrofurans. Tertiary alcohols, [symbol] , Secondary alcohols, Cyclic alcohols and derivatives, Oxacyclic compounds, Carboxylic acids, Dialkyl ethers, Organic oxides, Carbonyl compounds, Hydrocarbon derivates |
| Fumitomamide | 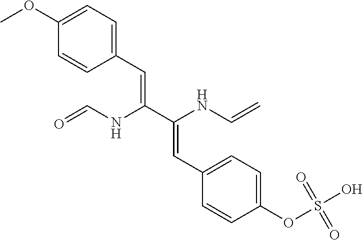 [SMILES: C=CNC(=C\c1ccc(OS(=O)(=O)O)cc1)/C(=C/c1ccc(OC)cc1)NC=O] | Lignans. [symbol] Methoxybenzenos Anisoles, Alkyl aryl ethers, Sulfuric acid monoesters, [symbol] , compounds, organic oxides, Hydrocarbon derivatives |
| Fumifungin | 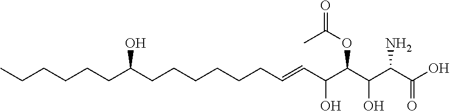 [SMILES: CCCCCC[C@@H](O)CCCCCC/C=C/C(O)[C@@H](OC(C)=O)C(O)[C@H](N)C(=O)O] | Lipids and lipid-like molecules. Fatty Acylis, Fatty acids and conjugates, Long-chain fatty acids, L-alpha-amino acids, Hydroxy fatty acids, Beta hydroxy acids and derivatives, Amino fatty acids, Unsaturated fatty acids, Dicarboxylic acids and derivatives, Secondary alcohols, Carboxylic acid esters, Amino acids, Polyols, Carboxylic acids, [symbol] compounds, Organic oxides, Monoalkylamines, Hydrocarbon derivatives, Carbonyl compound |
| Fumigaclavine C | 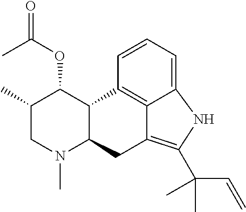 [SMILES: C=CC(C)(C)c1[nH]c2cccc3c2c1C[C@@H]1[C@@H]3[C@@H](OC(C)=O)[C@@H](C)CN1C] | Alkaloids and derivatives. Ergoline and derivatives, Clavinas and derivatives, Indoloquinolines, Benzoquinolines, Pyrroloquinolines, 3-alkylindoles [symbol] and derivatives, Acalkylamines, Substituted , [symbol] Amino acids and derivatives, Carboxylic acid estors, Monocarboxylic acids and derivatives, Azacyclc compounds, Carbonyl compounds, Hydrocarbon derivatives, Onganic oxides, Organopnictogen compounds |
| Fumigalonin | 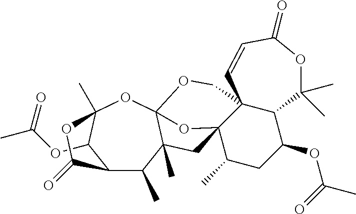 | Lipids and lipid-like molecules. Prenol lipids, Sesquiterpenoids, Abscisic acids and derivatives, Terpene lactones, Tetracarboxylic acids and derivatives, [symbol] Ketsis, Carboxylic acid orthoesters, Gamma butyrolactones, [symbol] , Enoate esters, Oxocyclic compounds, Organic oxides, Hyrdrocarbon derivatives, Carbonyl compounds |
| Fumigatoside B | 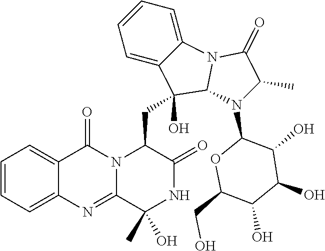 [SMILES: C[C@H]1C(=O)N2c3ccccc3[C@@](O)(C[C@H]3C(=O)N[C@](C)(O)c4nc5ccccc5c(=O)n43)[C@H]2N1[C@@H]1O[C@H](CO)[C@@H](O)[C@H](O)[C@H]1O] | Organic oxygen compounds. Organooxygen compounds, Carbohydrates and carbohydrate corjugat Glycoxyl compounds, Glycosylamines, Hexoses, Quinazetines, Alpha amino acids and derivatives, Indoles and derivatives, [symbol] , Tertiary carboxylic acid amides, Tertiary alcohols, Heteroarmatic compounds, Cyclic carboximidic acid Secondary alcohols, Lactams, Heriaminals, Propargylatype 1,3-dipolar Polyols, Oxacyclic compounds, Azacyclic compounds, Primary alcohols, Organopnictogen compounds, Organic oxides, Hydrocarbon derivatives, Carbonyl compounds |
| Fumiquinazoline A | 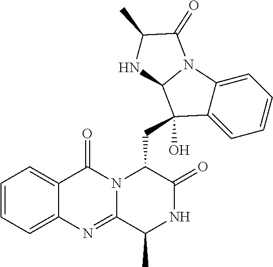 [SMILES: C[C@@H]1N[C@H]2N(C1=O)c1ccccc1[C@@]2(O)C[C@@H]1C(=O)N[C@@H](C)c2nc3ccccc3c(=O)n21] | Organoheterocyclic compounds. Diazanaphtheteres.Benzodiazines, Quinazolines, Alpha amino acids and derivatives, Indoles and derivatives, Pyrimidones, Imidazolidinones. Benzenoids, Tertiary carboxylic acid amides, Tertiary alcohols, Heteroaromatic compounds. Lactams, Secondary carboxylic acid amides, Dialkylamines, Azacyclic compounds, Organopnictogen compounds, Organic oxides, Hydrocarbon derivatives, Carbonyl compounds |
| Fumiquinone A | 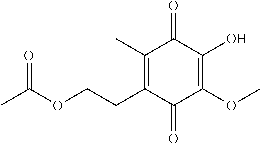 [SMILES: COC1=C(O)C(=O)C(C)=C(CCOC(C)=O)C1=O] | Lipids and lipid-like molecules. Prenol lipids, Quinone and hydroquinone lipids, Prenylquinones, Ubiquinones, P-benzoquinones, Vinylogous esters, Vinylogous acids, Carboxylic acid esters, Monocarboxylic acids and derivatives, Organic oxides, Hydrocarbon derivatives |
| Fumisoquin A | 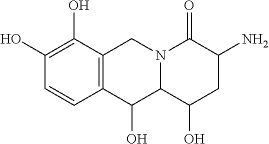 [SMILES: NC1CC(O)C2C(O)c3ccc(O)c(O)c3CN2C1=O] | Organoheterocyclic compounds. Tetahydrisoquinolines, Alpha amino acids and derivatives. Pipendinoes, Delta lactams, Azalkylamines, Aminopipetidines, 1-hydroxy-4-unsubstitited benzenoid 1-hydroxy-2-unsubstituted [symbol] , Tertiary carboxylic acid amides. Secondary alcohols, Polyols, Azacyclic compounds. Organopnictrogen compounds, Organic oxides. Monoalkylamines, Hydrocarbon derivatives, Carbonyl compounds |
| Fumetramorgin A | 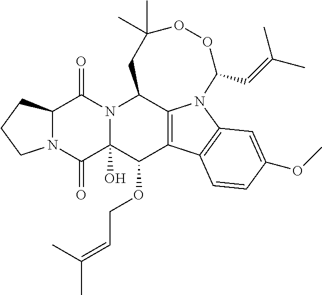 [SMILES: COc1ccc2c3c4n(c2c1)[C@@H](C=C(C)C)OOC(C)(C)C[C@@H]4N1C(=O)[C@@H]2CCCN2C(=O)[C@]1(O)[C@H]3OCC=C(C)C] | Organoheterocyclic compounds. Indoles and derivatives, [symbol] . Alpha amino acids and derivatives, 2,5-dioxopiperazines, Anisoles, Alkyl aryl ethers, N-alkylpiperazines. Heteroaromatic compounds, Pyrroles, Tertiary carboxylic acid amides, Pyrrodidines, Lactams, Oalkyl peroxides, Olalkyl ethers, Azacyclic compounds, Oxacyclic compounds, Akanolamines, Hydrocarbon derivatives, Carbonyl compounds, Organopnictogen compounds |
| [symbol] | 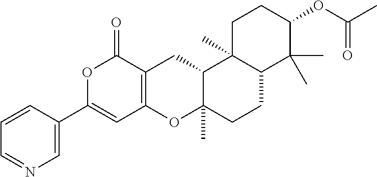 | Organoheterocyclic compounds. Naphthopyrans, Naphthalenes, Alkyl aryl ethers, Pyranores and [symbol] Pyridines and derivatives, Vinylogous esters, Hetoroaromatic compounds, Lactones, Carboxylic acid [symbol] Oxacyclic compounds, Monocarboxylic acids and derivatives, Azaoyclic compounds, Organic [symbol] , Hydrocarbon derivatives, Carbonyl compounds, Organonitrogen compounds, Organopnictogen [symbol] |
| [symbol] | 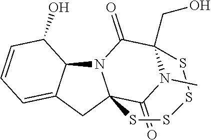 [SMILES: CN1C(=O)[C@]23CC4=CC=C[C@H](O)[C@H]4N2C(=O)[C@@]1(CO)SSSS3] | Organic acids and derivatives. Carboxylic acids and derivatives, Amino acids peptides, and analogues, Amino acids and derivatives, Alpha amino acids and derivatives, Thiodioxopiperzines, Indoles and derivatives N-methypiperazines, Tertiary carboxylic acid amides, Pyrrolidines, Secondary alcohols, Lactams, Axacyclic compounds, Primary alcohols, Organonitrogen compounds, Organic aides, Hydrocarbon derivatives, Carbonyl compounds |
| Hexadehydrossiechrome | 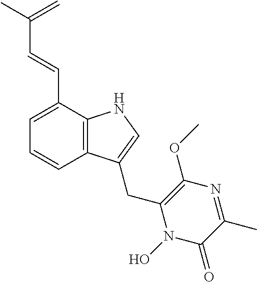 [SMILES: C=C(C)/C=C/c1cccc2c(Cc3c(OC)nc(C)c(=O)n3O)c[nH]c12] | Organoheteracyclic compounds. Indoles and derivatives, Indoles, 3-alkylindoles, Styrenes, Methoxypyrazines, Alkyl aryl ethers, [symbol] pyrroles, [symbol] compounds, Lactams, Organic transion metal salts, Azaryclic compounds, Organopnictogen compounds, Organonitrogen compounds, Organic oxides, Hydrocarbon derivatives |
| | 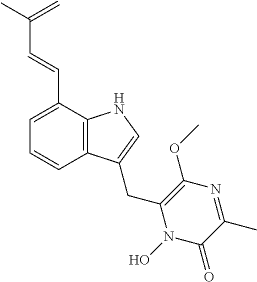 [SMILES: C=C(C)/C=C/c1cccc2c(Cc3c(OC)nc(C)c(=O)n3O)c[nH]c12] | |
| | 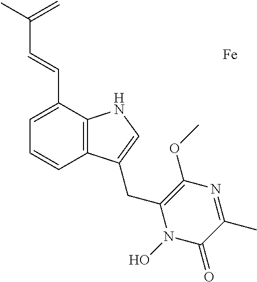 | |
| Isochaetominine | 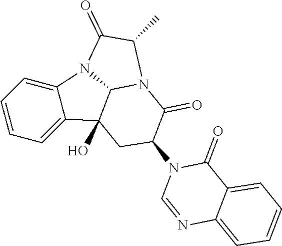 [SMILES: C[C@H]1C(=O)N2c3ccccc3[C@@]3(O)C[C@H](n4cnc5ccccc5c4=O)C(=O)N1[C@@H]23] | Organoheterocyclic compounds. Indoles and derivatives, Pyridoinodoles, Pyridoindolones, Alpha [symbol] Quinazolines, Alpha amino acids and derivatives, Indoles, [symbol] , Piperidiones, Delta lactams, Pyridines and derivatives, [symbol] Tertiary alcohols, Hetercaromatic compounds, Azacyclic compounds, Organognictogen compounds, Organonitagen compounds, Organic oxides, Hydrocarbon derivatives, Carbonyl compounds |
| Naosartorin | 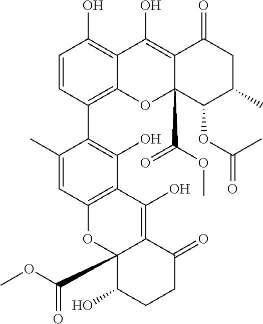 | Organoheterocyclic compounds. Benzopyrans, 1-henzopyrans, Dibenzonynans, Xanthenes, Tricarboxylic acids and derivatives [symbol] , Hydroxy acids and derivatives, Alkyl aryl ethers, 1-hydroxy-4-unsubstituted benzenoids, 1-hydroxy-2-unsubstituted benzenoids, Vinylognus acids, Methyl esters, Secondary alcohols, Ketones, Cyclic alcohols and derivatives, Polyols, Oxacyclic compounds, Enols, Organic oxides, Hydrocarbon derivatives |
| Patientoside A | 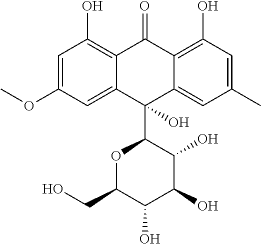 | Benzenoids. [symbol] Aryl ketones, 1-hydroxy-2-unsubstituted benzenoids, 1-hydroxy-4-unsubstituted benzenoids, Alkyl aryl ethers, [symbol] , Polyols, Oxacyclic compounds, Dialkyl ethers, Organic oxides, Hydrocarbon derivatives, Primary alcohols |
| Pyripyropene B | 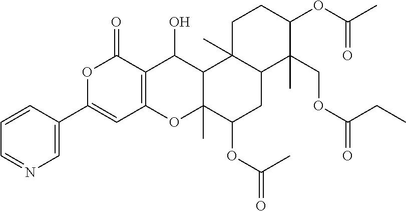 [SMILES: CCC(=O)OCC1(C)C(OC(C)=O)CCC2(C)C1CC(OC(C)=O)C1(C)Oc3cc(-c4cccnc4)oc(=O)c3C(O)C21] | Lipid and lipid-like molecules. Steroids and steroid derivatives, Hydrocycsteroids, 1-hydroxysteroids, Naphthopyrans, Naphthalenes, [symbol] acids and derivatives, Akyl aryl ethers, Pyranones and derivatives, Pyridines and derivatives, Vinylogous esters, [symbol] Oxacyclic compounds, Azacyclic compounds, Organic oxides, Hydrocarbon derivatives, Carbonyl compounds, Organonitrogen compounds, Organonictogon compounds |

[symbol]

indicates data missing or illegible when filed

Diversification of the Equisetin Scaffold Inferred from Gene Cluster Families

To further explore the link between metabolite scaffolds as represented by molecular and gene cluster families, the decalin-tetramic acids were examined, a structural class well represented in our BGC and metabolite datasets. This structural class, including compounds such as equisetin, altersetin, phomasetin, and trichosetin (Fig. BS11) (Refs. B31-B33; incorporated by reference in their entireties), has a wide range of reported biological activities, including antibiotic, anti-cancer, phytotoxic, and HIV integrase inhibitory activity (Ref. B34; incorporated by reference in its entirety). It was reasoned that further exploration of the decalin-tetramic acid structural class would yield insights into the biosynthetic mechanisms for variation of this bioactive scaffold by BGCs within the GCF.

Two closely related GCFs were identified (HYBRIDS_11/HYBRIDS_610) containing known BGCs responsible for biosynthesis of equisetin (Ref. B35; incorporated by reference in its entirety), trichosetin (Ref. B36; incorporated by reference in its entirety), and phomasetin (Ref. B37; incorporated by reference in its entirety) as well as BGCs from Alternaria likely responsible for the biosynthesis of altersetin found in multiple Alternaria species (Refs. B32, B38; incorporated by reference in their entireties). While most fungal GCFs are confined to single species or genera (Fig. B2), the equisetin GCF has an exceptionally broad phylogenetic distribution, with clusters found in the four Pezizomycotina classes Eurotiomycetes, Dothideomycetes, Xylonomycetes, and Sordariomycetes (Fig. B3B, left). The associated equisetin MF is likewise found in a variety of Dothideomycetes and Sordariomycetes (Fig. B3B, right).

The equisetin biosynthetic pathway involves three major steps: assembly of a decalin core via the action of polyketide synthase (PKS) enzyme domains and a Diels Alderase, formation of an amino acid-derived tetramic acid moiety catalyzed by NRPS domains, and N-methylation of the tetramic acid moiety (Fig. BS12) (Refs. B37, B39; incorporated by reference in their entireties). While the domain structure of the PKS contained in the equisetin GCF remains consistent across fungi, differences in backbone enzyme amino acid sequence and the presence/absence of tailoring enzymes mediate structural variations to the scaffold. The PKS enzymes from Fusarium oxysporum and Pyrenochaetopsis sp. RK10-F058 share 50% sequence identity, which likely result in the additional ketide unit and C-methylation observed in equisetin vs. phomasetin (Fig. B3B). In the NRPS module of the hybrid NRPS-PKS, changes to adenylation domain substrate binding residues are predicted to mediate incorporation of serine (trichosetin, equisetin, and phomasetin) and threonine (altersetin). The Aspergillus desertorum BGC contains adenylation domain substrate binding residues that are highly variant from those found in other clusters within the GCF, indicating its tetramic acid moiety is likely diversified with a different amino acid. The equisetin GCF contains additional variations in the number of enoyl reductase enzymes (one additional in the uncharacterized Penicillium expansum clade), indicating possible differences to degree of saturation, and a methyltransferase that is expected to mediate changes in tetramic acid N-methylation.

This pattern of biosynthetic variation within a GCF resulting in metabolite diversification indicates that exploring such pairs of GCFs and MFs with knowledge of their taxonomic distribution will be valuable to guide genome mining in the identification of new analogs of compounds with proven therapeutic or agrochemical value. The equisetin GCF is one of only 90 GCFs (representing 0.75% of total GCFs) within our dataset that spanned multiple taxonomic classes (Table 6). This includes bioactive scaffolds such PR-toxin, swainsonine, chaetoglobosin, and cytochalasin (Fig. BS13) which contain variations in tailoring enzyme composition expected to diversify these scaffolds. Given the observed biosynthetic diversity within such “multi-class” GCFs, exploring such pairs of GCFs and MFs represents an attractive approach for discovering new analogs of bioactive metabolites.

TABLE 6
The 90 gene cluster families (from total n = 12,067) that are exceptional in that they span
multiple taxonomic classes. The Reference column indicates a single GenBank accession number
and organism for the backbone enzyme. In cases of multiple backbone enzymes, the provided GenBank
reference corresponds to the backbone enzyme in bold text. Abbreviations are as follows: DHONTB,
dihydroxy-6-[(3E,5E,7E)-2-oxonona-3,5,7-trienyl]-benzaldehyde; HAS, hexadehydroastechrome;
KS, ketosynthase, AT, acyltransferase; DH, dehydratase; ER, enoyl reductase; KR, ketoreductase;
MT, methyltransferase; SAT, starter acyltransferase; PT, product template; A, adenylation; T,
thiolation; R, reductase; C, condensation; ICS, isocyanide synthase; DMAT, dimethylallyltransferases;
NRPS, nonribosomal peptide synthetase; PKS, polyketide synthase; HRPKS, highly reducing polyketide
synthase; NRPKS, nonreducing polyketide synthase; E, Eurotiomycetes; L, Leotiomycetes; S, Sodariomycetes;
D, Dothidiomycetes; X, Xylonomycetes; LEC, Lecanoromycetes.

| | | | TAXONOMIC |
|---|---|---|---|
| GCF | REFERENCE | BACKBONE | CLASSES |
| HRPKS_30 (DHONTB) | Aspergillus nidulans | PKS (KS-AT-DH-ER-KR-T), | E, S |
| | FGSC A4 (CBF86052) | PKS (SAT-KS-AT-PT-T-R) | |
| NRPKS_1343 | Fusarium fujikuroi | PKS (KS-AT-PT-T-TE) | L, S |
| (BIKAVERIN) | (SCO46930.) | | |
| NRPKS_791 | Cadophora sp. DSE1049 | PKS (SAT-KS-AT-PT-T-T-TE) | D, L, S |
| (MELANIN) | (PVH73815) | | |
| NRPS_607 | Aspergillus lentulus | PKS (KS-AT-DH-MT-ER-KR-T- | D, E |
| (CHAETOGLOBOSIN) | (GAQ05296) | C-A-T-R) | |
| NRPS_63 | Aspergillus arachidicola | NRPS (C-A-T-C-A-T-C) | E, S |
| (CHRYSOGINE) | (PIG85941) | | |
| NRPKS_375 | Aspergillus sydowii CBS | PKS (SAT-KS-AT-PT-T) | E, L, LEC, S |
| (CONIDIAL YELLOW | 593.65 (OJJ57401) | | |
| PIGMENT) | | | |
| NRPS_690 | Aspergillus clavatus | PKS (KS-AT-DH-MT-ER-KR-T- | E, L, S |
| (CYTOCHALASIN) | NRRL 1 (EAW09117) | C-A-T-R) | |
| NRPS_138 | Alternaria alternata | PKS (KS-AT-DH-MT-ER-KR-T- | D, E, S |
| (EQUISETIN) | (OWY46706) | C-A-T-R) | |
| NRPS_123 | Aspergillus fumigatus | NRPS (A-T-C-A-T-C) | E, L, S |
| (FUMITREMORGIN) | (OXN23238) | | |
| NRPS_1705 | Fusarium verticillioides | PKS (KS-AT-DH-MT-ER-KR-T) | D, S |
| (FUMONISIN) | (RBR13858) | | |
| NRPS_442 (HAS) | Aspergillus fumigatus | NRPS (A-T-C-A-T-C-T) | E, S |
| | (OXN25028) | | |
| NRPKS_147 | Alternaria alternata | PKS (SAT-KS-AT-PT-T-T-TE) | D, E |
| (MELANIN) | (OAG24502) | | |
| NRPS_101 | Aspergillus clavatus | PKS (KS-AT-DH-MT-ER-KR-T- | D, E, S, X |
| (PHOMASETIN) | NRRL 1 (EAW07624) | C-A-T-R) | |
| NRPS_1149 | Metarhizium acridum | NRPS (A-T-C-C-A-T-C-A-T-C- | L, S |
| (SERINOCYCLIN) | CQMa 102 (EFY85053) | A-T-C-C-A-T-C-C-A-T-C-C-A- | |
| | | T-C) | |
| HYBRIDS_151 | Clohesyomyces aquaticus | PKS (A-T-KS-AT-KR-T-R) | E, S |
| (SWAINSONINE) | (ORY11783) | | |
| NRPS_2042 | Oidiodendron maius Zn | PKS (KS-AT-DH-MT-ER-KR-T- | L, S |
| (UCS1025A) | (KIM94019) | C-A-T-R) | |
| DMAT_140 | Ophiocordyceps | DMAT | E, S |
| | australis (PHH64516) | | |
| DMAT_401 | Colletotrichum | PKS (SAT-KS-AT-MT-PT-T-TE), | L, S |
| | orchidophilum (OHF04557) | DMAT | |
| DMAT_411 | Cadophora sp. DSE1049 | DMAT | L, S |
| | (PVH84683) | | |
| HRPKS_1152 | Meliniomyces bicolor E | PKS (KS-AT-DH-MT-ER-KR-T) | L, S |
| | (PMD61012) | | |
| HRPKS_128 | Pezoloma ericae | PKS (KS-AT-DH-MT-ER-KR-T) | E, L |
| | (PMD17755) | | |
| HRPKS_1289 | Acremonium | PKS (KS-AT-DH-MT-ER-KR-T- | L, S |
| | chrysogenum ATCC | Carnitine_acyltransferase) | |
| | 11550 (KFH46614) | | |
| HRPKS_1318 | Colletotrichum | PKS (KS-AT-DH-MT-ER-KR- | L, S |
| | higginsianum IMI | T-C), PKS (KS-AT-DH-MT- | |
| | 349063 (OBR06526) | ER-KR-T) | |
| HRPKS_159 | Penicillium griseofulvum | PKS (SAT-KS-AT-PT-MT-R), | E, S |
| | (KXG49005) | PKS (KS-AT-DH-MT-ER-KR-T) | |
| HRPKS_170 | Penicillium camemberti | PKS (KS-AT-DH-MT-ER-KR-T) | E, L |
| | (CRL31088) | | |
| HRPKS_216 | Aspergillus sydowii CBS | NRPS (C-A-T-C-C-A-T-C-A-T- | E, L |
| | 593.65 (OJJ61536) | C-A-T-C) | |
| HRPKS_495 | Aspergillus uvarum CBS | PKS (KS-AT-DH-ER-KR-T) | E, S |
| | 121591 (PYH83208) | | |
| HRPKS_53 | Colletotrichum | PKS (KS-AT-DH-ER-KR-T-R) | E, S |
| | chlorophyti (OLN93260) | | |
| HRPKS_597 | Cordyceps sp. RAO-2017 | PKS (KS-AT-DH-ER-KR-T) | E, S |
| | (PHH90746) | | |
| HRPKS_678 | Pseudogymnoascus sp. | PKS (KS-AT-DH-MT-ER-KR-T- | E, L |
| | VKM F-3557 | Carnitine_acyltransferase) | |
| | (KFX86927) | | |
| HRPKS_694 | Phialocephala subalpina | PKS (KS-AT-DH-MT-ER-KR-T) | E, L |
| | (CZR67900) | | |
| HRPKS_882 | Fusarium fujikuroi | PKS (KS-AT-DH-MT-ER-KR-T) | S, X |
| | (SCN83763) | | |
| HYBRIDS_195 | Aspergillus | PKS (KS-AT-DH-MT-ER-KR-T- | E, S |
| | ochraceoroseus IBT | C-A-T-R) | |
| | 24754 (PTU20620) | | |
| HYBRIDS_215 | Penicillium camemberti | PKS (KS-AT-DH-MT-ER-KR-T) | E, S |
| | (CRL19370) | | |
| HYBRIDS_506 | Talaromyces stipitatus | PKS (KS-AT-DH-MT-ER-KR-T) | E, L |
| | ATCC 10500 (EED18841) | | |
| HYBRIDS_9 | Penicillium | PKS (KS-AT-DH-MT-ER-KR-T- | D, E, L |
| | subrubescens (OKP00032) | C-A-T-R) | |
| NRPKS_1290 | Pseudogymnoascus sp. | PKS (KS-AT-DH-MT-ER-KR-T-C) | E, L |
| | 05NY08 (OBT71831) | | |
| NRPKS_1320 | Coniochaeta pulveracea | PKS (KS-AT-DH-ER-KR-T) | E, S |
| | (RKU46359) | | |
| NRPKS_1782 | Phialocephala | PKS (SAT-KS-AT-PT-T-T-TE), | D, L |
| | scopiformis (KUJ09200) | PKS (DH-KR) | |
| NRPKS_1988 | Pseudogymnoascus sp. | PKS (SAT-KS-AT-PT-T) | D, L |
| | 23342-1-11 (OBT65120) | | |
| NRPKS_20 | Pseudogymnoascus sp. | PKS (KS-AT-DH-MT-ER-KR-T- | |
| | VKM F-103 (KFY80205) | C-A-T-R) | |
| NRPKS_250 | Aspergillus lentulus | PKS (KS-AT-DH-ER-KR-T) | E, L |
| | (GAQ09994) | | |
| NRPKS_437 | Aspergillus kawachii | PKS (A-T-KS-AT-KR-T-R) | |
| | IFO 4308 (GAA83965) | | |
| NRPKS_447 | Endocarpon pusilium | PKS (A-T-KS-AT-KR-T-R) | |
| | Z07020 (ERF68696) | | |
| NRPKS_5 | Penicillium nalgiovense | PKS (SAT-KS-AT-PT-T) | E, X |
| | (OQE96240) | | |
| NRPKS_510 | Trichoderma asperellum | PKS (SAT-KS-AT-PT-T) | E, S |
| | CBS 433.97 (PTB35070) | | |
| NRPKS_548 | Fusarium oxysporum f. | PKS (KS-AT-DH-MT-KR-T-C- | |
| | sp. cepae (RKK07595) | A-T-R) | |
| NRPKS_604 | Scedosporium | PKS (KS-AT-DH-MT-KR-T-R) | E, S |
| | apiospermum (KEZ41293) | | |
| NRPKS_787 | Penicillium griseofulvum | PKS (KS-AT-DH-MT-ER-KR-T-R) | D, E, L |
| | (KXG49279) | | |
| NRPS_1018 | Pseudogymnoascus sp. | NRPS (A-T-C-A-T-R) | D, L |
| | VKM F-3808 | | |
| | (KFX99775) | | |
| NRPS_1055 | Bipolaris victoriae FI3 | PKS (KS-AT-DH-MT-ER-KR-T- | D, L |
| | (EUN25091) | C-A-T-R) | |
| NRPS_1064 | Coleophoma | PKS (SAT-KS-AT-PT-T-T-TE), | E, L |
| | cylindrospora | NRPS (C-A-T) | |
| | (RDW81833) | | |
| NRPS_111 | Aspergillus brasiliensis | PKS (KS-AT-DH-MT-ER-KR- | E, S |
| | CBS 101740 (OJJ75537) | T-C-A-T-R), NRPS (A-T-C) | |
| NRPS_1222 | Talaromyces stipitatus | PKS (KS-AT-DH-MT-ER-KR-T), | E, S |
| | ATCC 10500 | NRPS (A-T-C-A-T-C-T) | |
| | (EED13058) | | |
| NRPS_1295 | Penicillium steckii | PKS (KS-AT-DH-MT-ER-KR-T- | E, L, S |
| | (OQE21884) | C-A-T-R) | |
| NRPS_1301 | Aspergillus bombycis | PKS (KS-AT-DH-ER-KR-T-C-A- | E, S |
| | (OGM48141) | T-R) | |
| NRPS_1372 | Fusarium avenaceum | PKS (KS-AT-DH-MT-ER-KR-T- | E, S |
| | (KIL86455) | C-A-T-R) | |
| NRPS_1410 | Helicocarpus griseus | PKS (KS-AT-DH-MT-ER-KR-T- | E, S |
| | UAMH5409 (PGH19023) | C-A-T-R) | |
| NRPS_1417 | Aspergillus | PKS (KS-AT-DH-MT-ER-KR-T- | E, L |
| | heteromorphus CBS | C-A-T-R) | |
| | 117.55 (PWY81896) | | |
| NRPS_151 | Madurella mycetomatis | PKS (KS-AT-DH-MT-ER-KR-T- | |
| | (KXX75968) | C-A-T-R) | |
| NRPS_1545 | Fusarium avenaceum | PKS (KS-AT-DH-ER-KR-T), | E, L, S |
| | (KIL87829) | NRPS (T-C-A-T-C-A-T-C-A- | |
| | | T-C-T) | |
| NRPS_1559 | Pseudogymnoascus sp. | PKS (KS-AT-DH-MT-ER-KR-T- | E, L |
| | VKM F-3775 (KFY27678) | C-A-T-R) | |
| NRPS_1586 | Metarhizium rileyi RCEF | NRPS (A-T-C-A-T-C-A-T-R) | E, S |
| | 4871 (OAA34246) | | |
| NRPS_2023 | Colletotrichum | PKS (KS-AT-DH-MT-ER-KR-T- | D, S |
| | graminicola M1.001 | C-A-T-R) | |
| | (EFQ35223) | | |
| NRPS_2636 | Bipolaris sorokiniana | NRPS (A-T-C) | E, S |
| | ND90Pr (EMD59100) | | |
| NRPS_283 | Aspergillus bombycis | PKS (KS-AT-DH-ER-KR-T-C-A- | E, S |
| | (OGM44044) | T-R) | |
| NRPS_353 | Beauveria bassiana | PKS (KS-AT-DH-MT-KR-T-C-A- | E, S |
| | ARSEF 2860 (EJP61198) | T-R) | |
| NRPS_41 | Aspergillus steynii IBT | NRPS (A-T-C-A-T-C) | E, L |
| | 23096 (PLB43453) | | |
| NRPS_457 | Coleophoma crateriformis | PKS (KS-AT-DH-MT-ER-KR-T), | E, L |
| | (RDW59260) | NRPS (A-T-C) | |
| NRPS_480 | Capronia coronata CBS | NRPS (A-T-C-T-C) | E, L |
| | 617.96 (EXJ78804) | | |
| NRPS_514 | Aspergillus mulundensis | PKS (KS-AT-DH-MT-ER-KR-T), | E, S, L |
| | (RDW86494) | NRPS (T-C-A-T-C-A-T-C-A- | |
| | | T-C-A-T-C) | |
| NRPS_569 | Cladophialophora | NRPS (A-T-C-A-T-C-T-C-A-T-C- | E, L |
| | carrionii (OCT48933) | T-C-T-C) | |
| NRPS_648 | Hypoxylon sp. CO27-5 | PKS (KS-AT-DH-MT-ER-KR- | E, S |
| | (OTA94984) | T-C-A-T-R), NRPS (A-T-R) | |
| NRPS_777 | Cordyceps fumosorosea | PKS (KS-AT-DH-MT-ER-KR-T- | E, S |
| | ARSEF 2679 | C-A-T-R) | |
| | (OAA69787) | | |
| NRPS_871 | Aspergillus fischeri | NRPS (A-T-C-A-T-R), | D, E, L, S |
| | NRRL 181 (EAW20390) | NRPS (A-T-C-A-T-C) | |
| NRPS_932 | Aspergillus costaricaensis | PKS (KS-AT-DH-MT-ER-KR-T), | D, E, L |
| | CBS 115574 (RAK83302) | PKS (KS-AT-DH-MT-ER-KR-T) | |
| NRPSLIKE_10 | Aspergillus | NRPS-like (ICS-A-T-Transferase) | D, E, S |
| | ochraceoroseus | | |
| | (KKK21469) | | |
| NRPSLIKE_1029 | Cladophialophora | NRPS-like (A-T-R) | E, L |
| | carrionii CBS 160.54 | | |
| | (ETI26263) | | |
| NRPSLIKE_11 | Aspergillus lentulus | NRPS-like (ICS-A-T-Transferase) | E, S |
| | (GAQ04120) | | |
| NRPSLIKE_1277 | Amorphotheca resinae | NRPS-like (A-T-R) | L, S |
| | ATCC 22711 (PSS07172) | | |
| NRPSLIKE_128 | Exophiala oligosperma | NRPS-like (A-T-Transferase) | D, S |
| | (KIW43198) | | |
| NRPSLIKE_1465 | Neonectria ditissima | NRPS-like (A-T-R) | D, S |
| | (KPM46454) | | |
| NRPSLIKE_1739 | Ophiocordyceps australis | NRPS-like (A-T-TE) | D, L, S |
| | (PHH75740) | | |
| NRPSLIKE_22 | Cladophialophora | NRPS-like (A-T-R-DH) | |
| | bantiana CBS 173.52 | | |
| | (KIW93789) | | |
| NRPSLIKE_266 | Penicillium occitanis | NRPS-like (A-T-R), | E, L |
| | (PCG97091) | NRPS-like (A-T-R) | |
| NRPSLIKE_869 | Cladophialophora | NRPS-like (A-T-R) | E, L |
| | bantiana CBS 173.52 | | |
| | (KIW89508) | | |
| NRPSLIKE_873 | Cladophialophora | NRPS-like (A-T-R) | E, L |
| | carrionii CBS 160.54 | | |
| | (ETI24620) | | |
| NRPSLIKE_899 | Talaromyces marneffei | NRPS-like (A-T-R) | E, L |
| | ATCC 18224 (EEA18553) | | |
| TERPENE_1140 | Exserohilum turcica | trichodiene synthase | D, L |
| | Et28A (EOA88708) | | |
| TERPENE_139 | Penicillium camemberti | terpene cyclase | E, S |
| | (CRL18805) | | |

TABLE 7
Protein domain rules for classifying gene clusters as nonribosomal
peptide synthase (NRPS), highly-reducing polyketide synthase (HR-
PKS), nonreducing polyketide synthase (NR-PKS) hybrid NRPS-PKS,
NRPS-like, dimethylallyl transferase (DMAT), or terpene.

| | | DOMAINS |
|---|---|---|
| BGC TYPE | DOMAINS PRESENT | ABSENT |
| NRPS | Adenylation, condensation | N/A |
| HR-PKS | Ketosynthase, dehydratase | N/A |
| NR-PKS | Ketosynthase and product | N/A |
| | template or starter | |
| | acyltransferase | |
| HYBRID | Adenylation, ketosynthase | N/A |
| NRPS-PKS | | |
| NRPS-LIKE | Adenylation | Condensation |
| DMAT | Dimethylallyl transferase | N/A |
| | Terpene synthase, | |
| | terpene cyclase, | |
| TERPENE | trichodiene synthase, or | N/A |
| | polyprenyl synthetase | |

### Comparing the Fungal Versus Bacterial Biosynthetic Space

Having surveyed GCFs across the fungal kingdom, experiments were conducted during development of embodiments herein to compare and contrast this genomic and chemical repertoire to the well-established bacterial canon. 5,453 bacterial genomes whose BGCs were publicly available in the antiSMASH bacterial BGCs database (Ref. B40; incorporated by reference in its entirety) were gathered, resulting in a dataset of 24,024 bacterial BGCs to compare to the dataset of 36,399 fungal BGCs. To visualize the biosynthetic space encompassed by these BGCs, the frequency of protein domains within BGCs for each major taxonomic group was determined. Principle Component Analysis (PCA) of these encoded BGCs showed a phylogenetic bias in this biosynthetic space, with bacteria and fungi occupying distinct regions (Fig. B4A).

Dramatic differences in bacterial versus fungal NRPS and PKS assembly line logic were observed. Consistent with prior studies of iterative fungal PKS enzymes (Ref. B41; incorporated by reference in its entirety), fungal PKS BGCs typically encode a single backbone PKS enzyme, while bacterial PKS BGCs contain a median of 1.7 PKS backbone enzymes per cluster (Fig. B4B, right). Fungal NRPS BGCs also usually encode a single backbone enzyme, compared to multiple backbone enzymes more typically observed in bacterial systems (Fig. B4B, left). Fungal NRPS and PKS enzymes also average ˜150% the size of bacterial backbones (Fig. BS14). In addition to these contrasting backbone enzyme compositions, systematic differences were observed in the top NRPS domain organizations (Fig. BS15), particularly in NRPS termination domains (Fig. B4C). The most common fungal NRPS termination domains are C-terminal condensation domains, recently found to catalyze release of peptide intermediates via intramolecular cyclization (Refs. B42-B44; incorporated by reference in their entireties). The next most common are terminal thioester reductase domains that perform either reductive release to aldehydes or alcohols or release via cyclization (Ref. B45; incorporated by reference in its entirety). This is in stark contrast to bacterial NRPS BGCs, which most commonly terminate with type I thioesterase domains that release intermediates as linear or cyclic peptides (Fig. B4C).

These collective differences between fungal and bacterial BGCs show systematic differences in NRPS biosynthetic logic between these two kingdoms. In bacterial NRPS canon, a pathway is comprised of multiple NRPS genes whose chromosomal order (and the order of catalytic domain “modules” within the encoded polypeptide) corresponds to the order of amino acid monomers in the metabolite product (Fig. B4D, right) (Ref. B46; incorporated by reference in its entirety). In the field of bacterial natural products, the use of this “collinearity rule” to predict metabolite scaffolds is commonplace (Refs. B19, B47, B48; incorporated by reference in their entireties); however, the large number of exceptions to this rule reduces the accuracy of these predictions. The prototypical fungal NRPS (Fig. B4D (FIG. B4D) primarily involves the action of biosynthetic domains within the same backbone enzyme, rather than multiple NRPS backbones acting in concert. This indicates that efforts to predict fungal NRPS scaffolds will be able to largely bypass the need to account for permutations of multiple NRPS genes, raising the possibility of increased predictive performance compared to bacteria.

### Uncovering Distinct Natural Product Reservoirs

Having shown that fungi and bacteria are distinct biosynthetically, experiments were conducted during development of embodiments herein to compare these genomics-based insights to the chemical space of known metabolites. 9,382 bacterial compounds were added to the dataset of 15,213 fungal metabolites, analyzing these bacterial compounds using the same network analysis and chemical ontology workflow described above. PCA was performed to visualize the chemical space of major fungal and bacterial taxonomic groups within this compound dataset.

PCA of bacterial and fungal compounds (Fig. B5A) revealed a trend that parallels the analysis of fungal and bacterial biosynthetic space (Fig. B4A). Bacteria and fungi occupy separate regions of chemical space, differing dramatically in terms of chemical ontology superclass, a high-level descriptor of general structural type (Fig. B5B). Fungi have twice the frequency of lipids and nearly twice the frequency of heterocyclic compounds, a structural group that includes aromatic polyketide-related moieties such as furans and pyrans. Many of the chemical moieties and structural classes that are highly enriched in bacteria or fungi are vital in bioactive scaffolds. This includes moieties such as the bacterial aminoglycoside antibiotics (Ref. B49; incorporated by reference in its entirety), thiazoles present in the bacterial anti-cancer bleomycin family (Ref. B50; incorporated by reference in its entirety), and the steroid ring that forms the core scaffold of steroid drugs such as the fungal metabolite fusidic acid (Ref. B51; incorporated by reference in its entirety) (Fig. B5B). PCA loadings plots similarly reveal differences between bacterial and fungal chemical space, including a high prevalence of peptide-associated chemical ontology terms in bacteria, and lipid and aromatic polyketide terms in fungi (Fig. BS16).

Within the fungal kingdom, differences in PCA of the chemical repertoire of major taxonomic groups were observed (Fig. BS17). Pezizomycotina classes grouped together in chemical space, largely due to a higher proportion of polyketide and peptide-related chemical moieties (Fig. BS18). Basidiomycota are distinct chemically, possessing a much higher proportion of chemical moieties and descriptors associated with terpenes and other lipids. These observations based on chemical space are consistent with the higher proportion of NRPS and PKS BGCs within Pezizomycotina and the prevalence of terpene BGCs within Basidiomycota groups such as Agaricomycotina (Fig. B2B), and further supported by PCA of fungal BGCs, in which fungal phyla represent distinct groups (Figs. BS19 and BS20).

### A Framework for Exploring Fungal Scaffolds Using Gene Cluster Families

The GCF approach enables the systematic mapping of the biosynthetic repertoire encoded by large groups of fungal genomes. The fungal kingdom is a wealth of untapped biosynthetic potential, with the 1000 genomes analyzed here representing a reservoir of >12,000 new GCF-encoded scaffolds. This genome dataset is only a small subset of the >1 million predicted fungal species (Ref. B29; incorporated by reference in its entirety), indicating that the total biosynthetic potential of the fungal kingdom far surpasses that assembled here.

By organizing biosynthetically related BGCs into families, the GCF approach provides a means of cataloguing and dereplicating genome-encoded MFs. In the field of bacterial natural products discovery, this GCF paradigm has been expanded for automated linking of GCFs to MFs detected by metabolomics and molecular networking analysis, enabling high-throughput genome mining from industrial-scale strain collections (Refs. B5, B7, B29, B52; incorporated by reference in their entireties). Establishing the GCF approach for fungal genomes lays the groundwork for similar GCF-driven large-scale compound discovery efforts from fungi.

### Data-Driven Prospecting for Fungal Natural Products

Large-scale genome sequencing projects such as the 1000 Fungal Genomes project, whose stated goal is sampling every taxonomic family within Fungi (Ref. B53; incorporated by reference in its entirety), will uncover a large amount of biosynthetic and chemical novelty. However, as 76% of fungal GCFs are species- and 16% are genus-specific, such genome sequencing efforts focused on taxonomic families will miss the majority of GCFs. Additional large-scale efforts to sample this biosynthetic space based on “depth” rather than “breadth” is suggested to more efficiently access these genomes. Future projects, now feasible for academic research groups due to ever-decreasing genome sequencing costs, should focus on expanding this dataset with species-level sequencing of taxonomic groups.

The GCF approach provides a means of selecting fungi for compound and BGC discovery via approaches such as heterologous expression (Ref. B54; incorporated by reference in its entirety) based not on taxonomic or phylogenetic markers, but with a strategy that focuses on efficient sampling of biosynthetic pathways. The distribution of GCFs shows groups of organisms with shared GCFs (Fig. BS6), and sampling based on these organism “groups” reduces the number of genomes required to capture the majority of fungal biosynthetic space. Simulated sampling based on shared GCFs indicated that 80% of GCFs from the 386 Eurotiomycete genomes are represented in a sample of only 145 genomes. By contrast, to represent the same number of GCFs, species-level sampling required 189 genomes and random sampling required 263 genomes (Fig. BS21). This indicates that the GCF approach provides a roadmap for systematic characterization of new fungal biosynthetic pathways and their compounds.

### Unearthing New Medicines

Analyses of both chemical and biosynthetic space show that bacteria and fungi represent chemically distinct sources for natural products discovery. Fungal compounds are closer to FDA-approved compounds than bacterial compounds in terms of several chemical properties, including three out of four “Lipinsky Rule of Five” properties often used as guidelines for predicting oral bioavailability (Fig. BS22) (Ref. B55; incorporated by reference in its entirety). While many of the most successful natural products violate these rules of thumb, these data indicate that fungal metabolites may be more “druglike” than those occupying bacterial chemical space.

Compound discovery efforts should be initiated with the understanding that different biological sources will yield distinct chemical space and different types of metabolite scaffolds. The fungal kingdom is rich in aromatic polyketides, while bacteria harbor a higher proportion of peptidic scaffolds. Within the fungal kingdom, Basidiomycota is a rich reservoir of terpene scaffolds, while BGC-rich Pezizomycotina classes are a richer source of polyketides and peptides. These data indicate that distinct taxonomic groups not only possess the capacity for different metabolite scaffolds, but also different types of scaffolds.

### Strain Selection Based on PCR Markers

Rather than strain selection with the goal of maximizing biodiversity (i.e., the stated purpose of the 1000 Fungal Genomes Project), experiments were conducted during development of embodiments herein for selection of strains based on an optimal degree of overlap in genetic content. The approach requires strains to have some BGCs in common; however, also seeks biosynthetic diversity. A goal is to establish an optimal pipeline for strain selection for linked genomics & metabolomics, and offer the study below of genetic markers as a proxy for GCF overlap in fungal strains.

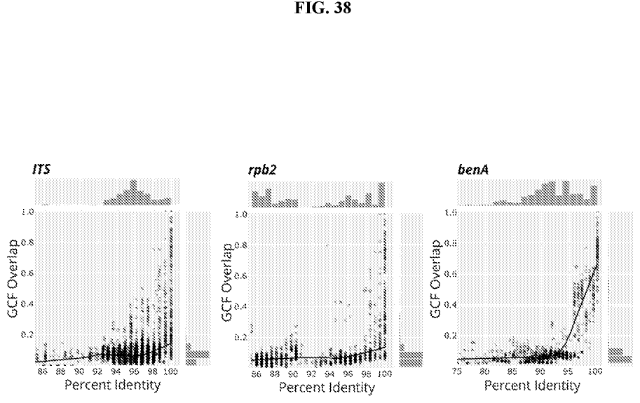

From 1037 fungal genomes, a set of ˜12,000 GCFs was generated and the relationship between GCF similarity and genetic markers was determined. To find genetic marker sequences that could be used as a proxy for GCF overlap in selection of fungal strains, the GCF overlap was plotted vs. three genetic markers that have been previously used for fungal phylogeny (FIG. 38). ITS (internal transcribed spacer) is the most commonly used genetic marker for fungi; however, many strains have identical ITS sequences but very little GCF overlap. Similarly, the rpb2 gene (RNA polymerase subunit B), another proposed fungal genetic marker, also results in many strains that are identical by rpb2 but with essentially no GCF overlap. In contrast, the beta tubulin gene (benA) shows a clear relationship with GCF overlap, with distances of 96-99% benA identity corresponding to 40-60% GCF overlap (FIG. 38). Therefore, these data support the use of benA as a high-quality marker for GCF overlap in selected strains. Thus, PCR amplification of ITS, rpb2, and benA genes are performed for ˜20 trial strains in the very beginning of the granting period, using previously reported primers. The three markers are compared based on PCR success rate and amplicons will be sequenced using simple Sanger sequencing. After this optimization, a final primer set is deployed on ˜2-fold more strains than are selected. This involves PCR on genomic DNA from ˜500 strains, after which the final 250 are selected for full interrogation by metabologenomics.

### Preliminary Metabologenomics Data on 50 Strains of Fungi.

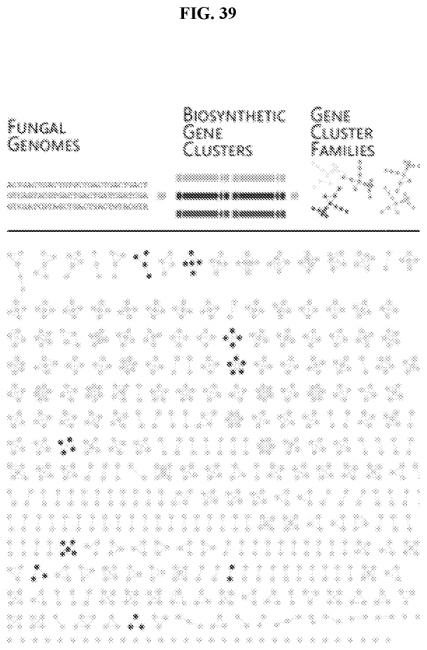

Experiments were conducted during development of embodiments herein to establish a new fungal bioinformatics pipeline (FIG. 39, top) based on the bioinformatics workflows described here. This workflow involves detection of biosynthetic gene clusters using antiSMASH and organization of gene clusters into fungi-specific biosynthetic classes (NRPS, HR-PKS, NR-PKS, NRPS-like, etc.) based on their protein domain composition. A series of pairwise comparisons is then performed using a distance metric based on the fraction of shared protein domains and domain sequence similarity. The weighted sum of these two metrics is used as a combined similarity metric for clustering, resulting in a biosynthetic network of 594 GCFs expected to produce highly similar metabolites. To produce a preliminary dataset, this workflow was used to organize 50 Aspergillus and Penicillium genomes into a network of GCFs (FIG. 39, bottom). This GCF approach enables visualization of the “biosynthetic space” of a strain collection. Annotation of gene clusters based on similarity to knowns allows for targeted discovery of new analogs of compounds with proven value.

The second component of the platform combines state-of-the-art HRMS mass spectrometry with a cheminformatics pipeline for dereplication of known compounds in metabolite extracts. UHPLC-MS metabolomics data was collected for the same 50 Aspergillus and Penicillium strains analyzed using our GCF analysis workflow. Each strain was grown on four media conditions for expression of diverse metabolites. Metabolite extracts were analyzed using an Agilent 1290 UHPLC and Q Exactive mass spectrometer dedicated to natural product extract analysis. Metabolomics data was analyzed using molecular networking, an approach that clusters spectra from related metabolites into molecular families for data visualization and annotation.

The pipeline uses a metabologenomics approach to connect GCFs to their metabolite products for discovery of new compounds and biosynthetic enzymes. The presence/absence of GCFs and molecular families across a strain collection are compared using a chi-squared test, and statistically significant correlations represent putative biosynthetic relationships. These data are visualized using the Prospect web application (prospect-fungi.com/) that allow targeting of specific GCFs and metabolites for further characterization.

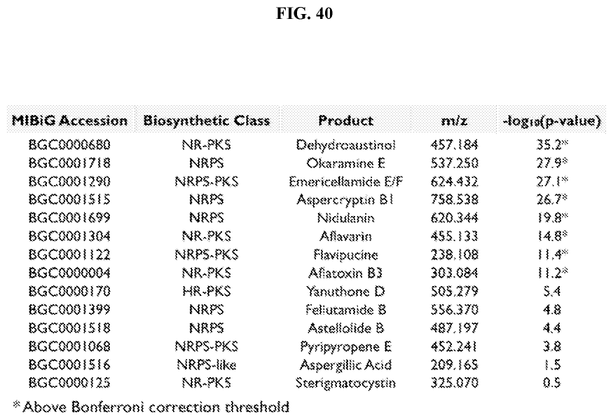

Using 50 strains of Aspergillus and Penicillium, a set of 14 experimentally characterized fungal GCFs were examined from the database MIBiG whose metabolite products were detected. After applying the conservative Bonferroni approach to estimate the False Discovery Rate (FDR) and correct for multiple hypothesis testing, statistically-significant correlations for 8/14 knowns was observed, a success rate of ˜60% (FIG. 40).

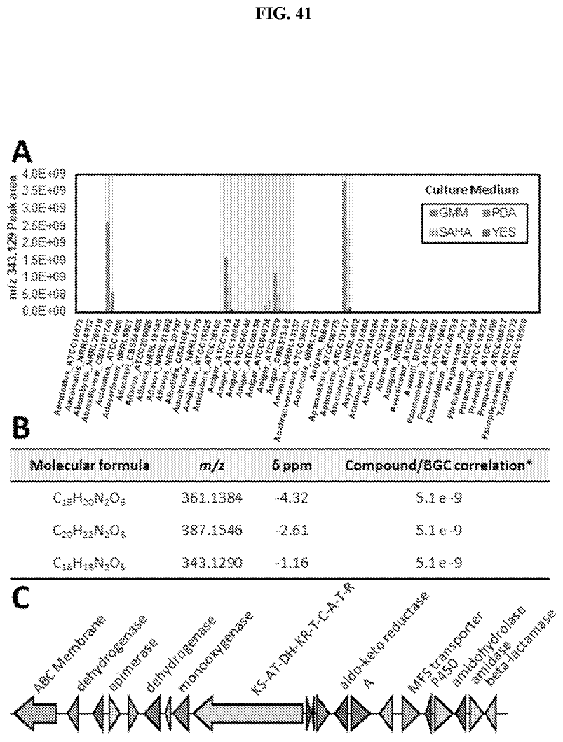

Experiments will be conducted during development of embodiments herein to expand the fungal metabolomics dataset with, minimally, an additional 250 Aspergillus, Penicillium, and Eurotiales strains, resulting in a total of 300 for this project. Metabolomics data from these strains are annotated using an improved version of this molecular networking cheminformatics pipeline and correlated to biosynthetic pathways as demonstrated here in FIGS. 39-41. These data will be integrated to create an annotated library of NP/BGC pairs, including both previously known and new pairs for follow-up characterization (e.g., shown in FIG. 41, below).

### Implementation Via Prospect

Experiments conducted during development of embodiments herein have led to the creation of a web tool known as Prospect which provides a variety of views and a page that allows users to browse BGCs in each of the GCFs we have assigned to date. This includes a side panel that displays all gene clusters present within the family, with genes color-coded by detected protein domains. Compounds associated with experimentally characterized clusters are also visible in this alpha-version of Prospect. Upon selecting a specific gene, a page shows detected protein domains, with links to relevant Pfam database entries and the option to download or perform an NCBI BLAST search with a protein or domain sequence. In addition to this page for viewing GCFs, additional pages display tables allowing users to find GCFs based on taxonomy information, Prospect accession number, biosynthetic type, and experimentally characterized status.

The alpha version of Prospect was designed using a combination of programming frameworks and languages chosen based on their ability to scale to large datasets, their level of creator/developer support, their ability to provide interactive user experiences, and their proven track record and popularity with web developers. The frontend visual component was designed using Angular, a framework commonly used in enterprise software development that is designed by and heavily supported by Google. The backend, responsible for accessing a SQL database housing all genomics and metabolomics data, was designed as a RESTful API using Django, a Python framework with strong community support used by organizations such as Instagram, Mozilla, and NASA.

### Correlative Identification of a New NP BGC Pair in 5 Aspergilli

Using the process above on 50 strains of phylogenetically diverse fungi from the Aspergillus and Penicillium genera, FIG. 40 shows anchoring of the method using 8 knowns. Among these 50 strains, 594 gene cluster families were identified. Expression screening using HRMS led to the detection of 8914 ions contained within these extracts, the majority of which have neither been characterized nor linked to their biosynthetic machinery. The 8914 ions were organized into 998 molecular families using spectral networking. Within just the dataset of 50 strains, 80 new NP/BGC pairs were detected with p-values <0.001 after Bonferroni correction. One such NP/BGC pair is described below.

Correlative analysis highlighted the gene cluster family “hybrids_158”; of the 9 strains that have one of the 9 BGCs in this GCF, their expression of a compound detected by mass spec as an ion at 343.129 m z is shown in FIG. 41, panel A. This gene cluster family contains a large backbone gene with both PKS and NRPS modules, and several tailoring enzymes and transporters that apparently play a role in its biosynthesis (FIG. 41C). Of the 9 strains that contained this gene cluster, 5 of them produced a set of three related secondary metabolites based on mass spectral fragmentation patterns, each of which correlated to the hybrids_158 GCF with a p-value of 5.1×10⁻⁹ (significant after Bonferroni multiple hypothesis correction) (FIG. 41B). Both the molecular formulas and MS fragmentation patterns for these ions support the presence of both polyketide and peptide components and affirms this compound is not present in our database of ˜25,000 natural products (just over 14,000 of which are annotated as deriving from fungi). These 3 compounds were produced most abundantly in Aspergillus brasiliensis CBS 101740, which is being scaled up for compound isolation, heavy isotope-labeled by metabolic feeding studies of amino acids, and targeted cloning to both confirm the association of these ions to the gene cluster of interest and to elucidate the biosynthetic pathway for these molecules.

### REFERENCES

The following references, some of which are cited above by number, are incorporated herein by reference in their entireties.
1: Ernst M, Kang K B, Caraballo-Rodriguez A M, Nothias L F, Wandy J, Chen C, Wang M, Rogers S, Medema M H, Dorrestein P C, van der Hooft J J J. MolNetEnhancer: Enhanced Molecular Networks by Integrating Metabolome Mining and Annotation Tools. Metabolites. 2019 Jul. 16; 9(7). pii: E144. doi: 10.3390/metabo9070144. PubMed PMID: 31315242.
2: Rogers S, Ong C W, Wandy J, Ernst M, Ridder L, van der Hooft J J J. Deciphering complex metabolite mixtures by unsupervised and supervised substructure discovery and semi-automated annotation from MS/MS spectra. Faraday Discuss. 2019 May 23. doi: 10.1039/c8fd00235e. [Epub ahead of print] PubMed PMID: 31120050.
3: Dührkop K, Fleischauer M, Ludwig M, Aksenov A A, Melnik A V, Meusel M, Dorrestein P C, Rousu J, Bocker S. SIRIUS 4: a rapid tool for turning tandem mass spectra into metabolite structure information. Nat Methods. 2019 April; 16(4):299-302. doi: 10.1038/s41592-019-0344-8. Epub 2019 Mar. 18. PubMed PMID: 30886413.
4: Chevrette M G, Aicheler F, Kohlbacher O, Currie C R, Medema M H. SANDPUMA: ensemble predictions of nonribosomal peptide chemistry reveal biosynthetic diversity across Actinobacteria. Bioinformatics. 2017 Oct. 15; 33(20):3202-3210. doi: 10.1093/bioinformatics/btx400. PubMed PMID: 28633438; PubMed Central PMCID: PMC5860034.
5: Dührkop K, Shen H, Meusel M, Rousu J, Böcker S. Searching molecular structure databases with tandem mass spectra using CSI:FingerID. Proc Natl Acad Sci USA. 2015 Oct. 13; 112(41):12580-5. doi: 10.1073/pnas.1509788112. Epub 2015 Sep. 21. PubMed PMID: 26392543; PubMed Central PMCID: PMC4611636.
6: Doroghazi J R, Albright J C, Goering A W, Ju K S, Haines R R, Tchalukov K A, Labeda D P, Kelleher N L, Metcalf W W. A roadmap for natural product discovery based on large-scale genomics and metabolomics. Nat Chem Biol. 2014 November; 10(11):963-8. doi: 10.1038/nchembio.1659. Epub 2014 Sep. 28. PubMed PMID: 25262415; PubMed Central PMCID: PMC4201863
7: Nguyen D D, Wu C H, Moree W J, Lamsa A, Medema M H, Zhao X, Gavilan R G, Aparicio M, Atencio L, Jackson C, Ballesteros J, Sanchez J, Watrous J D, Phelan V V, van de Wiel C, Kersten R D, Mehnaz S, De Mot R, Shank E A, Charusanti P, Nagarajan H, Duggan B M, Moore B S, Bandeira N, Palsson BØ, Pogliano K, Gutiérrez M, Dorrestein P C. MS/MS networking guided analysis of molecule and gene cluster families. Proc Natl Acad Sci USA. 2013 Jul. 9; 110(28):E2611-20. doi: 10.1073/pnas.1303471110. Epub 2013 Jun. 24. PubMed PMID: 23798442; PubMed Central PMCID: PMC3710860
8: Röttig M, Medema M H, Blin K, Weber T, Rausch C, Kohlbacher O. NRPSpredictor2—a web server for predicting NRPS adenylation domain specificity. Nucleic Acids Res. 2011 July; 39(Web Server issue):W362-7. doi: 10.1093/nar/gkr323. Epub 2011 May 9. PubMed PMID: 21558170; PubMed Central PMCID: PMC3125756
9: Frank A M, Bandeira N, Shen Z, Tanner S, Briggs S P, Smith R D, Pevzner P A. Clustering millions of tandem mass spectra. J Proteome Res. 2008 January; 7(1):113-22. Epub 2007 Dec. 8. PubMed PMID: 18067247; PubMed Central PMCID: PMC2533155.
A1. Cragg G M, Newman D J. 2013. Natural products: a continuing source of novel drug leads. BBA-Gen Subjects 1830: 3670-3695.
A2. Cragg G M, Pezzuto J M. 2016. Natural products as a vital source for the discovery of cancer chemotherapeutic and chemopreventive agents. Med Prin Pract 25: 41-59.
A3. Newman D J, Cragg G M. 2016. Natural products as sources of new drugs from 1981 to 2014. J Nat Prod 79: 629-661.
A4. Roemer T, Xu D, Singh S B, Parish C A, Harris G, Wang H, Davies J E, Bills G F. 2011. Confronting the challenges of natural product-based antifungal discovery. Chem Biol 18: 148-164.
A5. Pelaez F. 2005. Biological activities of fungal metabolites, p. 41-92. In An Z. (ed), Handbook of Industrial Mycology, vol. 22, Marcel Dekker, New York.
A6. Keller N P, Turner G, Bennett J. 2005. Fungal secondary metabolism—from biochemistry to genomics. Nat Rev Microbiol 3: 937-947.
A7. Schueffler A, Anke T. 2014. Fungal natural products in research and development. Nat Prod Rep 31: 1425-1448.
A8. Li Y F, Tsai K J, Harvey C J, Li J J, Ary B E, Berlew E E, Boehman B L, Findley D M, Friant A G, Gardner C A. 2016. Comprehensive curation and analysis of fungal biosynthetic gene clusters of published natural products. Fungal Genet Biol 89: 18-28.
A9. Bok J W, Ye R, Clevenger K D, Mead D, Wagner M, Krerowicz A, Albright J C, Goering A W, Thomas P M, Kelleher N L, Keller N P, Wu C C. 2015. Fungal artificial chromosomes for mining of the fungal secondary metabolome. BMC Genomics 16: 343.
A10. Clevenger K D, Bok J W, Ye R, Miley G P, Verdan M H, Velk T, Chen C, Yang K, Robey M T, Gao P, Lamprecht M, Thomas P M, Islam M N, Palmer J M, Wu C C, Keller N P, Kelleher N L. 2017. A scalable platform to identify fungal secondary metabolites and their gene clusters. Nat Chem Biol 13: 895.
A11. Clevenger K D, Ye R, Bok J W, Thomas P M, Islam M N, Miley G P, Robey M T, Chen C, Yang K, Swyers M, Wu C C, Keller N P, Kelleher N L. 2018. Interrogation of benzomalvin biosynthesis using fungal artificial chromosomes with metabolomic scoring (FAC-MS): discovery of a benzodiazepine synthase activity. Biochemistry 57: 3237-3243.
A12. Robey M T, Ye R, Bok J W, Clevenger K D, Islam M N, Chen C, Gupta R, Swyers M, Wu E, Gao P, Thomas P M, Wu C C, Keller N P, Kelleher N L. 2018. Identification of the first diketomorpholine biosynthetic pathway using FAC-MS technology. ACS Chem Biol 13: 1142-1147.
A13. Fatokun A A, Hunt N H, Ball H J. 2013. Indoleamine 2, 3-dioxygenase 2 (IDO2) and the kynurenine pathway: characteristics and potential roles in health and disease. Amino Acids 45: 1319-1329.
A14. Jacobs K R, Castellano-Gonzalez G, Guillemin G J, Lovejoy D B. 2017. Major developments in the design of inhibitors along the kynurenine pathway. Curr Med Chem 24: 2471-2495.
A15. Giessen T W, Kraas F I, Marahiel M A. 2011. A four-enzyme pathway for 3, 5-dihydroxy-4-methylanthranilic acid formation and incorporation into the antitumor antibiotic sibiromycin. Biochemistry 50: 5680-5692.
A16. Zhang C, Yang Z, Qin X, Ma J, Sun C, Huang H, Li Q, Ju J. 2018. Genome mining for mycemycin: discovery and elucidation of related methylation and chlorination biosynthetic chemistries. Org Lett 20: 7633-7636.
A17. Andersen M R, Nielsen J B, Klitgaard A, Petersen L M, Zachariasen M, Hansen T J, Blicher L H, Gotfredsen C H, Larsen T O, Nielsen K F. 2013. Accurate prediction of secondary metabolite gene clusters in filamentous fungi. Proc Natl Acad Sci USA 110: E99-E107.
A18. Klitgaard A, Nielsen J B, Frandsen R J, Andersen M R, Nielsen K F. 2015. Combining stable isotope labeling and molecular networking for biosynthetic pathway characterization. Anal Chem 87: 6520-6526.
A19. Miao V, Coeffet-LeGal M-F, Brian P, Brost R, Penn J, Whiting A, Martin S, Ford R, Parr I, Bouchard M. 2005. Daptomycin biosynthesis in Streptomyces roseosporus: cloning and analysis of the gene cluster and revision of peptide stereochemistry. Microbiology 151: 1507-1523.
A20. Hirose Y, Watanabe K, Minami A, Nakamura T, Oguri H, Oikawa H. 2011. Involvement of common intermediate 3-hydroxy-L-kynurenine in chromophore biosynthesis of quinomycin family antibiotics. J Antibiot 64: 117-122.
A21. Wong C T, Lam H Y, Li X. 2013. Effective synthesis of kynurenine-containing peptides via on-resin ozonolysis of tryptophan residues: synthesis of cyclomontanin B. Org Biomol Chem 11: 7616-7620.
A22. Nguyen K T, Ritz D, Gu J-Q, Alexander D, Chu M, Miao V, Brian P, Baltz R H. 2006. Combinatorial biosynthesis of novel antibiotics related to daptomycin. Proc Natl Acad Sci USA 103: 17462-17467.
A23. Steenbergen J N, Alder J, Thome G M, Tally F P. 2005. Daptomycin: a lipopeptide antibiotic for the treatment of serious Gram-positive infections. J Antimicrob Chemother 55: 283-288.
A24. Yeung A W, Terentis A C, King N J, Thomas S R. 2015. Role of indoleamine 2, 3-dioxygenase in health and disease. Clin Sci 129: 601-672.
A25. Gulbis J, Mackay M, Rivett D. 1990. Structures of three 1-benzazepine-2, 5-diones: cyclic derivatives of N-acyl kynurenines. Acta Crystallogr C 46: 829-833.
A26. Li H, Gilchrist C L M, Phan C-S, Lacey H J, Vuong D, Moggach S A, Lacey E, Piggot A M, Chooi Y-H. 2020. Biosynthesis of a New Benzazepine Alkaloid Nanagelenin A from Aspergillus nanangensis Involves an Unusual L-Kynurenine-Incorporating NRPS Catalyzing Regioselective Lactamization. J Am Chem Soc 142: 7145-7152.
A27. Choera T, Zelante T, Romani L, Keller N P. 2018. A multifaceted role of tryptophan metabolism and indoleamine 2, 3-dioxygenase activity in Aspergillus fumigatus-host interactions. Front Immunol 8: 1996.
A28. Yuasa H J, Ball H J. 2012. The evolution of three types of indoleamine 2, 3 dioxygenases in fungi with distinct molecular and biochemical characteristics. Gene 504: 64-74.
A29. Baccile J A, Le H H, Pfannenstiel B T, Bok J W, Gomez C, Brandenburger E, Hoffmeister D, Keller N P, Schroeder F C. 2019. Diketopiperazine formation in fungi requires dedicated cyclization and thiolation domains. Angew Chem 58: 14589-14593.
A30. Balibar C J, Walsh C T. 2006. GliP, a Multimodular Nonribosomal Peptide Synthetase in Aspergillus fumigatus, Makes the Diketopiperazine Scaffold of Gliotoxin. Biochemistry 45: 15029-15038.
A31. Schmidt-Dannert C. 2016. Biocatalytic portfolio of Basidiomycota. Curr Opin Chem Biol 31: 40-49.
A32. Brown D W, Adams T H, Keller N P. 1996. Aspergillus has distinct fatty acid synthases for primary and secondary metabolism. Proc Natl Acad Sci USA 93: 14873-14877.
A33. Cacho R A, Jiang W, Chooi Y-H, Walsh C T, Tang Y. 2012. Identification and Characterization of the Echinocandin B Biosynthetic Gene Clsuter from Emericella rugulosa NRRL 11440. J Am Chem Soc 134: 16781-16790.
A34. Keller N P. 2019. Fungal secondary metabolism: regulation, function, and drug discovery. Nat Rev Microbiol 17: 167-180.
A35. Gilchrist C L M, Li H, Chooi, Y-H. 2018. Panning for gold in mould: can we increase the odds for fungal genome mining? Org Biomol Chem 16: 1620-1626.
A36. Yeh H-H, Ahuja M, Chiang Y-M, Oakley C E, Moore S, Yoon O, Hajovsky H, Bok J-W, Keller N P, Wang C C C, Oakley B R. 2016. Resistance gene-guided genome mining: serial promoter exchanges in Aspergillus nidulans reveal the biosynthetic pathway for fellutamide B, a proteasome inhibitor. ACS Chem Biol 11: 2275-2284.
A37. Lin H-C, Chooi Y-H, Dhingra S, Xu W, Calvo A M, Tang Y. 2013. The Fumagillin Biosynthetic Gene Cluster in Aspergillus fumigatus Encodes a Cryptic Terpene Cyclase Involved in the Formation of β-trans-Bergamotene. J Am Chem Soc 135: 4614-4619.
A38. Prendergast G C, Malachowski Wp, DuHadaway J B, Muller A J. 2017. Discovery of IDO1 inhibitors: from bench to bedsite. Cancer Res 77: 6795-6811.
B1. L. Bullerman, Significance of mycotoxins to food safety and human health. J Food Prot 42, 65-86 (1979).
B2. G. F. Bills, J. B. Gloer, Biologically active secondary metabolites from the fungi. Microbiol Spectr, 1087-1119 (2017).
B3. Y. F. Li et al., Comprehensive curation and analysis of fungal biosynthetic gene clusters of published natural products. Fungal Genet Biol 89, 18-28 (2016).
B4. N. P. Keller, Fungal secondary metabolism: regulation, function and drug discovery. Nat Rev Microbiol 17, 167-180 (2019).
B5. D. D. Nguyen et al., MS/MS networking guided analysis of molecule and gene cluster families. Proc. Natl. Acad. Sci. USA 110, E2611-E2620 (2013).
B6. P. Cimermancic et al., Insights into secondary metabolism from a global analysis of prokaryotic biosynthetic gene clusters. Cell 158, 412-421 (2014).
B7. J. R. Doroghazi et al., A roadmap for natural product discovery based on large-scale genomics and metabolomics. Nat Chem Biol 10, 963 (2014).
B8. J. C. Navarro-Muñoz et al., A computational framework to explore large-scale biosynthetic diversity. Nat Chem Biol 16, 60-68 (2020).
B9. S. A. Kautsar, J. J. Van Der Hooft, D. De Ridder, M. H. Medema, BiG-SLiCE: A highly scalable tool maps the diversity of 1.2 million biosynthetic gene clusters. BioRxiv (2020).
B10. X.-L. Li et al., Rapid discovery and functional characterization of diterpene synthases from basidiomycete fungi by genome mining. Fungal Genet Biol 128, 36-42 (2019).
B11. S. Gao et al., Genome-wide analysis of Fusarium verticillioides reveals inter-kingdom contribution of horizontal gene transfer to the expansion of metabolism. Fungal Genet Biol 128, 60-73 (2019).
B12. I. Kærbolling, U. H. Mortensen, T. Vesth, M. R. Andersen, Strategies to establish the link between biosynthetic gene clusters and secondary metabolites. Fungal Genet Biol 130, 107-121 (2019).
B13. J. C. Nielsen et al., Global analysis of biosynthetic gene clusters reveals vast potential of secondary metabolite production in Penicillium species. Nat Microbiol 2, 1-9 (2017).
B14. K. Hoogendoorn et al., Evolution and diversity of biosynthetic gene clusters in Fusarium. Front Microbiol 9, 1158 (2018).
B15. S. Theobald et al., Uncovering secondary metabolite evolution and biosynthesis using gene cluster networks and genetic dereplication. Sci Rep 8, 1-12 (2018).
B16. K-S. Ju et al., Discovery of phosphonic acid natural products by mining the genomes of 10,000 actinomycetes. Proc Natl Acad Sci USA 112, 12175-12180 (2015).
B17. J. Y. Yang et al., Molecular networking as a dereplication strategy. J Nat Prod 76, 1686-1699 (2013).
B18. S. A. Cantrell, J. Dianese, J. Fell, N. Gunde-Cimerman, P. Zalar, Unusual fungal niches. Mycologia 103, 1161-1174 (2011).
B19. K. Blin et al., antiSMASH 4.0—improvements in chemistry prediction and gene cluster boundary identification. Nucleic Acids Res 45, W36-W41 (2017).
B20. N. Khaldi et al., SMURF: genomic mapping of fungal secondary metabolite clusters. Fungal Genet Biol 47, 736-741 (2010).
B21. I. Kjærbolling et al., Linking secondary metabolites to gene clusters through genome sequencing of six diverse Aspergillus species. Proc Natl Acad Sci USA 115, E753-E761 (2018).
B22. T. C. Vesth et al., Investigation of inter- and intraspecies variation through genome sequencing of Aspergillus section Nigri. Nat Genet 50, 1688-1695 (2018).
B23. F. A. Simão, R. M. Waterhouse, P. Ioannidis, E. V. Kriventseva, E. M. Zdobnov, BUSCO: assessing genome assembly and annotation completeness with single-copy orthologs. Bioinformatics 31, 3210-3212 (2015).
B24. M. H. Medema et al., Minimum information about a biosynthetic gene cluster. Nat Chem Biol 11, 625-631 (2015).
B25. D. Butina, Unsupervised data base clustering based on daylight's fingerprint and Tanimoto similarity: A fast and automated way to cluster small and large data sets. J Chem Inf Comput Sci 39, 747-750 (1999).
B26. C. R. Pye, M. J. Bertin, R. S. Lokey, W. H. Gerwick, R. G. Linington, Retrospective analysis of natural products provides insights for future discovery trends. Proc Natl Acad Sci USA 114, 5601-5606 (2017).
B27. J. A. Van Santen et al., The natural products atlas: an open access knowledge base for microbial natural products discovery. ACS Cent Sci 5, 1824-1833 (2019).
B28. Y. D. Feunang et al., ClassyFire: automated chemical classification with a comprehensive, computable taxonomy. J Cheminform 8, 61 (2016).
B29. M. Blackwell, The Fungi: 1, 2, 3 . . . 5.1 million species? Am J Bot 98, 426-438 (2011).
B30. A. W. Goering et al., Metabologenomics: correlation of microbial gene clusters with metabolites drives discovery of a nonribosomal peptide with an unusual amino acid monomer. ACS Cent Sci 2, 99-108 (2016).
B31. R. F. Vesonder, L. W. Tjarks, W. K. Rohwedder, H. R. Burmeister, J. A. Laugal, Equisetin, an antibiotic from Fusarium equiseti NRRL 5537, identified as a derivative of N-methyl-2,4-pyrollidone. J Antibiot (Tokyo) 32, 759-761 (1979).
B32. V. Hellwig et al., Altersetin, a New Antibiotic from Cultures of Endophytic Alternaria spp. J Antibiot (Tokyo) 55, 881-892 (2002).
B33. E. C. Marfori, S. i. Kajiyama, E.-i. Fukusaki, A. Kobayashi, Trichosetin, a novel tetramic acid antibiotic produced in dual culture of Trichoderma harzianum and Catharanthus roseus callus. Z Naturforsch C 57, 465-470 (2002).
34. R. Schobert, A. Schlenk, Tetramic and tetronic acids: an update on new derivatives and biological aspects. Bioorg Med Chem 16, 4203-4221 (2008).
B35. J. W. Sims, J. P. Fillmore, D. D. Warner, E. W. Schmidt, Equisetin biosynthesis in Fusarium heterosporum. Chem Commun, 186-188 (2005).
B36. S. Janevska et al., Establishment of the inducible Tet-on system for the activation of the silent trichosetin gene cluster in Fusarium fujikuroi. Toxins 9, 126 (2017).
B37. N. Kato et al., Control of the stereochemical course of [4+2] cycloaddition during trans-decalin formation by Fsa2-family enzymes. Angew Chem Int Ed Engl 130, 9902-9906 (2018).
B38. J. J. Kellogg et al., Biochemometrics for natural products research: comparison of data analysis approaches and application to identification of bioactive compounds. J Nat Prod 79, 376-386 (2016).
B39. X. Li, Q. Zheng, J. Yin, W. Liu, S. Gao, Chemo-enzymatic synthesis of equisetin. Chem Commun 53, 4695-4697 (2017).
B40. K. Blin et al., The antiSMASH database version 2: a comprehensive resource on secondary metabolite biosynthetic gene clusters. Nucleic Acids Res 47, D625-D630 (2019).
B41. C. D. Campbell, J. C. Vederas, Biosynthesis of lovastatin and related metabolites formed by fungal iterative PKS enzymes. Biopolymers 93, 755-763 (2010).
B42. X. Gao et al., Cyclization of fungal nonribosomal peptides by a terminal condensation-like domain. Nat Chem Biol 8, 823-830 (2012).
B43. J. A. Baccile et al., Diketopiperazine formation in fungi requires dedicated cyclization and thiolation domains. Angew Chem Int Ed Engl 58, 14589-14593 (2019).
B44. L. K. Caesar et al., Heterologous expression of the unusual terreazepine biosynthetic gene cluster reveals a promising approach for identifying new chemical scaffolds. mBio 11 (2020).
B45. M. W. Mullowney, R. A. McClure, M. T. Robey, N. L. Kelleher, R. J. Thomson, Natural products from thioester reductase containing biosynthetic pathways. Nat Prod Rep 35, 847-878 (2018).
B46. G. L. Challis, J. H. Naismith, Structural aspects of non-ribosomal peptide biosynthesis. Curr Opin Struct Biol 14, 748-756 (2004).
B47. M. A. Skinnider, N. J. Merwin, C. W. Johnston, N. A. Magarvey, PRISM 3: expanded prediction of natural product chemical structures from microbial genomes. Nucleic Acids Res 45, W49-W54 (2017).
B48. M. A. Skinnider et al., Genomes to natural products prediction informatics for secondary metabolomes (PRISM). Nucleic Acids Res 43, 9645-9662 (2015).
B49. K. M. Krause, A. W. Serio, T. R. Kane, L. E. Connolly, Aminoglycosides: an overview. Cold Spring Harb Perspec Med 6, a027029 (2016).
B50. U. Galm et al., Antitumor antibiotics: bleomycin, enediynes, and mitomycin. Chem Rev 105, 739-758 (2005).
B51. L. Verbist, The antimicrobial activity of fusidic acid. J Antimicrob Chemother 25, 1-5 (1990).
B52. A. W. Goering et al., Metabologenomics: correlation of microbial gene clusters with metabolites drives discovery of a nonribosomal peptide with an unusual amino acid monomer. ACS central science 2, 99-108 (2016).
B53. I. V. Grigoriev et al., MycoCosm portal: gearing up for 1000 fungal genomes. Nucleic Acids Res. 42, D699-D704 (2014).
B54. K. D. Clevenger et al., A scalable platform to identify fungal secondary metabolites and their gene clusters. Nat Chem Biol 13, 895 (2017).
B55. C. A. Lipinski, F. Lombardo, B. W. Dominy, P. J. Feeney, Experimental and computational approaches to estimate solubility and permeability in drug discovery and development settings. Adv Drug Deliv Rev 23, 3-25 (1997).

1. A method of combined genomic and metabolomic analysis comprising:
(a) analyzing genomic sequences from multiple strains of fungi to generate a network of biosynthetic gene clusters (BGCs);
(b) analyzing mass spectra of extracts from multiple strains of fungi to generate a network of metabolite features; and
(c) comparing the network of BGCs and network of metabolites to link particular mass spectrometric features with the BGCs responsible for the synthesis of metabolites that correspond to the particular mass spectrometric features.

2. The method of claim 1, wherein the genomic sequences from multiple strains of fungi comprise 100 or more full or partial genomic sequences.

3. The method of claim 1, wherein the genomic sequences from multiple strains of fungi comprise full or partial genomic sequences from 100 or more strains of fungi.

4. The method of claim 1, wherein the genomic sequences from multiple strains of fungi comprise full or partial genomic sequences from 100 or more species of fungi.

5. The method of claim 1, wherein analyzing genomic sequences from multiple strains of fungi comprises identifying BGCs with the genomic sequences.

6. The method of claim 1, wherein analyzing genomic sequences from multiple strains of fungi comprises grouping BGCs with the genomic sequences into gene cluster families (GCFs).

7. The method of claim 1, wherein analyzing genomic sequences from multiple strains of fungi is based on pairwise comparisons of sequence and predicted structural features of the BGCs.

8. The method of claim 1, wherein the mass spectra of extracts from multiple strains of fungi comprise 100 or more mass spectra.

9. The method of claim 1, wherein the mass spectra of extracts from multiple strains of fungi comprise mass spectra from 100 or more strains of fungi.

10. The method of claim 1, wherein the mass spectra of extracts from multiple strains of fungi comprise mass spectra from 100 or more species of fungi.

11. The method of claim 1, wherein analyzing mass spectra of extracts from multiple strains of fungi comprises identifying mass spectrometric features with the mass spectra.

12. The method of claim 1, wherein analyzing mass spectra of extracts from multiple strains of fungi comprises grouping mass spectrometric features with the mass spectra into molecular families (MFs).

13. The method of claim 1, wherein analyzing mass spectra of extracts from multiple strains of fungi is based on pairwise comparisons of mass spectrometric features of the mass spectra

14. The method of claim 1, wherein comparing the network of BGCs and network of metabolite features comprises comparing the pairwise distances of BGCs or GCFs within the BGC network with the pairwise distances of metabolite features or MFs within the metabolite feature network to identify correlations that indicate that a BGC or GCF is responsible for the synthesis of a metabolite feature or MF.

15. The method of claim 1, wherein comparing the network of BGCs and network of metabolite features comprises comparing the frequency of BGCs or GCFs within the BGC network with the frequency of metabolite features or MFs within the metabolite feature network to identify correlations that indicate that a BGC or GCF is responsible for the synthesis of a metabolite feature or MF.

16. A network linking metabolite features from 100 or more mass spectra of extracts from multiple strains of fungi with BGCs from 100 or more genomic sequences from multiple strains of fungi, wherein linking of a mass spectrometric feature with a BGC indicates that the BGC is involved in the synthesis of a metabolite that produced the mass spectrometric feature.

17. A method of fungal genomic analysis comprising:
(a) identifying biosynthetic gene clusters (BGCs) within genomic sequences from multiple strains of fungi;
(b) identifying sequence characteristics and predicted structural domains within the BGCs; and
(c) comparing the sequence characteristics and predicted structural domains between multiple pairs of BGCs to determine the degree of relatedness between the pairs of BGCs.

18. The method of claim 17, further comprising:
(d) generating a network of BGCs based on the degree of relatedness between the pairs of BGCs.

19. The method of claim 17, further comprising:
(d) generating grouping the BGCs into gene cluster families based on the degree of relatedness between the pairs of BGCs.

20. A method of fungal metabolomic analysis comprising:
(a) identifying mass spectrometric features within mass spectra of extracts from multiple strains of fungi;
(b) comparing characteristics of the mass spectrometric features between multiple pairs of mass spectrometric features to determine the degree of relatedness between the pairs of mass spectrometric features; and
(c) generating a network of mass spectrometric features based on the degree of relatedness between the pairs of mass spectrometric features.

21. The method of claim 20, further comprising:
(d) grouping the mass spectrometric features into molecular families based on the degree of relatedness between the pairs of mass spectrometric features.


SEQUENCE LISTING (24 sequences)

SEQ ID NO: 1 - protein, 10 aa, Artificial sequence
  Synthetic peptide
GIILFGVVTK

SEQ ID NO: 2 - protein, 10 aa, Artificial sequence
  Synthetic peptide
GVIFMAAGVK

SEQ ID NO: 3 - protein, 10 aa, Artificial sequence
  Synthetic peptide
GINFIGAGTK

SEQ ID NO: 4 - protein, 10 aa, Artificial sequence
  Synthetic peptide
GVIILAAGIK

SEQ ID NO: 5 - protein, 10 aa, Artificial sequence
  Synthetic peptide
GALFFAAGVK

SEQ ID NO: 6 - protein, 10 aa, Artificial sequence
  Synthetic peptide
GVIFMAAGVK

SEQ ID NO: 7 - protein, 10 aa, Artificial sequence
  Synthetic peptide
GIILFGVVTK

SEQ ID NO: 8 - protein, 10 aa, Artificial sequence
  Synthetic peptide
GIILFGVVTK

SEQ ID NO: 9 - protein, 10 aa, Artificial sequence
  Synthetic peptide
DAAMIMGIAK

SEQ ID NO: 10 - protein, 10 aa, Artificial sequence
  Synthetic peptide
DVLSFGASLK

SEQ ID NO: 11 - protein, 10 aa, Artificial sequence
  Synthetic peptide
DAWTTTGVGK

SEQ ID NO: 12 - protein, 10 aa, Artificial sequence
  Synthetic peptide
DAWTTTGVAK

SEQ ID NO: 13 - protein, 10 aa, Artificial sequence
  Synthetic peptide
DCGMSMGVGK

SEQ ID NO: 14 - protein, 10 aa, Artificial sequence
  Synthetic peptide
DCGMSMGVGK

SEQ ID NO: 15 - protein, 9 aa, Artificial sequence
  Synthetic peptide
SHAVADLNS

SEQ ID NO: 16 - protein, 9 aa, Artificial sequence
  Synthetic peptide
SHVVGDAAT

SEQ ID NO: 17 - protein, 9 aa, Artificial sequence
  Synthetic peptide
THALWDGGP

SEQ ID NO: 18 - protein, 12 aa, Artificial sequence
  Synthetic peptide
FYALGGDSIKAI

SEQ ID NO: 19 - protein, 12 aa, Artificial sequence
  Synthetic peptide
FRALGGHSVLQM

SEQ ID NO: 20 - protein, 12 aa, Artificial sequence
  Synthetic peptide
FFEAGGDSIQAW

SEQ ID NO: 21 - protein, 12 aa, Artificial sequence
  Synthetic peptide
FFHLGGDSVNGM

SEQ ID NO: 22 - protein, 12 aa, Artificial sequence
  Synthetic peptide
FFRLGGNSVRAL

SEQ ID NO: 23 - protein, 9 aa, Artificial sequence
  Synthetic peptide
  misc_feature (3)..(5): Xaa can be any naturally occurring amino acid
  misc_feature (7)..(8): Xaa can be any naturally occurring amino acid
  MISC_FEATURE (9)..(9): Xaa = S or T
SHXXXDXXX

SEQ ID NO: 24 - protein, 6 aa, Artificial sequence
  Synthetic peptide
  misc_feature (3)..(5): Xaa can be any naturally occurring amino acid
SHXXXD